(19) United States
(12) Patent Application Publication
(10) Pub. No.: US 2026/0218179 A1
(43) Pub. Date: Jul. 30, 2026

(54) COMPOSITIONS AND METHODS FOR MODULATING SOS GENE EXPRESSIONS

(71) Applicant: Molecular Axiom, LLC, San Diego, CA (US)

(72) Inventors: Yuching CHEN, San Diego, CA (US); Lishan CHEN, San Diego, CA (US); Bohan JIN, San Diego, CA (US)

(21) Appl. No.: 19/142,452

(22) PCT Filed: Dec. 22, 2023
(86) PCT No.: PCT/US2023/085707

§ 371 Date: Jun. 23, 2025

Related U.S. Application Data
(60) Provisional application No. 63/476,895, filed on Dec. 22, 2022.
(60) Provisional application No. 63/479,103, filed on Jan. 9, 2023.

(51) Int. Cl. C12N 15/113; A61P 35/00; C12N 15/88

(52) U.S. Cl. CPC C12N 15/113; A61P 35/00; C12N 15/88; C12N 2310/11; C12N 2310/313; C12N 2310/315; C12N 2310/321; C12N 2310/3231; C12N 2310/3341; C12N 2310/341; C12N 2310/3515

(57) ABSTRACT

Described herein are compositions for modulating gene expressions. Also described herein are methods for using the compositions described herein for modulating gene expressions.

### PRIORITY

This application claims the benefit of, and priority to, U.S. Provisional Application No. 63/476,895, filed Dec. 22, 2022, and U.S. Provisional Application No. 63/479,103, filed Jan. 9, 2023, the contents of which are hereby incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Certain diseases or conditions are caused by genetic mutations or deregulated signaling pathways due to over- or under-expression of one or more genes affecting the signaling pathway. To treat such diseases or conditions, one of the most sought after treatment option involves direct editing of the genetic mutations, or transcriptional/translational regulation using gene silencing tools or methods. Oligonucleotide-induced gene silencing can control RNA expression of target genes in various aspects including transcription inactivation, mRNA degradation, transcriptional attenuation. Therefore, there remains a need for compositions and methods for effective control of gene expression, and particularly for control of RNA levels.

### SUMMARY

Described herein, in some embodiments, is a composition comprising an antisense oligonucleotide capable of binding to son of sevenless 1 (SOS1) or son of sevenless 2 (SOS2) mRNA, wherein the antisense oligonucleotide comprises a nucleobase sequence having at least 80%, 85%, or 90% sequence identity (or 100% sequence identity) to one of the following sequences: SEQ ID NOs: 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 or 187. In some embodiments, the antisense oligonucleotide comprises a nucleobase sequence having at least 80%, 85%, or 90% sequence identity (or 100% sequence identity) to one of the following sequences: SEQ ID NOs: 16, 20, 27, 31, 42, 56, 65, 66, 73, 94, 95, 101, 107, 139, 158, or 187. Described herein, in some aspects, is a composition comprising an antisense oligonucleotide capable of binding to son of sevenless 1 (SOS1) or son of sevenless 2 (SOS2) mRNA, wherein the antisense oligonucleotide comprises a sequence having at least 8 consecutive nucleobases of one of the following sequences: SEQ ID NOs: 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 or 187. In some embodiments, the antisense oligonucleotide comprises a nucleobase sequence having at least 80%, 85%, or 90% sequence identity (or 100% sequence identity) to one of the following sequences: SEQ ID NOs: 16, 20, 27, 31, 42, 56, 65, 66, 73, 94, 95, 101, 107, 139, 158, or 187. In some embodiments, the antisense oligonucleotide specifically binds to the SOS1 mRNA. In some embodiments, the antisense oligonucleotide specifically binds to the SOS2 mRNA. In some embodiments, the antisense oligonucleotide downregulates mRNA expression of SOS1 or SOS2 by at least 30%. In some embodiments, the antisense oligonucleotide downregulates mRNA expression of SOS1 and SOS2 each by at least 30%. In some embodiments, the antisense oligonucleotide is 12-30 nucleotides in length. In some embodiments, the antisense oligonucleotide is a gapmer, comprising a gap segment and a wing segment. In some embodiments, the antisense oligonucleotide comprises 5′-wing segment and 3′-wing segment. In some embodiments, each of the 5′-wing segment and 3′-wing segment is independently from 2 to 5 (e.g., three or four) linked nucleotides. In some embodiments, the antisense oligonucleotide comprises at least one 2′-modified nucleoside, at least one modified internucleotide linkage, or at least one inverted abasic moiety. In some embodiments, the at least one 2′ modified nucleotide comprises: 2′-O-methyl, 2′-O-methoxyethyl(2′-O-MOE), 2′-O-aminopropyl, 2′-deoxy, 2′-deoxy-2′-fluoro, 2′-O-aminopropyl (2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′-O—N-methylacetamido (2′-O-NMA) modified nucleotide; comprises locked nucleic acid (LNA), constrained ethyl (cEt) sugar, or ethylene nucleic acid (ENA); or comprises a combination thereof. In some embodiments, the at least one modified internucleotide linkage comprises a phosphorothioate linkage or a phosphorodithioate linkage. In some embodiments, the antisense oligonucleotide comprises a phosphorodiamidate morpholino oligomer (PMO), locked nucleic acid (LNA) or constrained ethyl (cEt) sugar. In some embodiments, the antisense oligonucleotide is conjugated with a peptide, antibody, lipid, carbohydrate, or polymer. In some embodiments, the antisense oligonucleotide is conjugated with the peptide, antibody, lipid, carbohydrate, or polymer via a linker. In some embodiments, the composition comprises a combination of an antisense oligonucleotide that specifically binds to the SOS1 mRNA and an antisense oligonucleotide that specifically binds to the SOS2 mRNA. In some embodiments, the composition comprises an antisense oligonucleotide capable of binding to both the SOS1 mRNA and the SOS2 mRNA. In some embodiments, the composition further comprises an excipient. In some embodiments, the composition further comprises at least one active ingredient. In some embodiments, the composition is formulated for parenteral or nasal administration.

Described herein, in some aspects, is a method of modulating KRAS-mediated signaling pathway in a cancer cell in need of, comprising: treating the cancer cell with a composition comprising antisense oligonucleotide described herein, and capable of binding to son of sevenless 1 (SOS1) or son of sevenless 2 (SOS2) mRNA, thereby reducing expression of the SOS1 or SOS2 mRNA or expression of SOS1 or SOS2 protein in the cancer cell. In some embodiments, the cancer cell is a lung cancer cell, a pancreatic cancer cell, or a colon cancer cell. In some embodiments, the expression of the SOS1 or the SOS2 protein or mRNA is reduced at least 30%, at least 40%, at least 50% after the treatment. In some embodiments, the expression of each of the SOS1 and the SOS2 proteins or each of the SOS1 and the SOS2 mRNAs is each reduced at least 30%, at least 40%, at least 50% after the treatment.

Described herein, in some aspects, is a method of treating a genetic disorder in a subject in need thereof, the method comprising: administering the subject a composition comprising an antisense oligonucleotide described herein, thereby treating the genetic disorder in the subject. In some embodiments, the genetic disorder is Noonan syndrome. In some embodiments, the genetic disorder is hereditary gingival fibromatosis type 1.

Described herein, in some aspects, is a method of treating a cancer in a subject in need thereof, the method comprising: administering the subject a composition comprising an antisense oligonucleotide described herein, thereby treating the cancer in the subject. In some embodiments, the cancer is associated with an abnormality of KRAS-mediated signaling pathway. In some embodiments, the cancer is a lung cancer, a pancreatic cancer, or a colon cancer. In some embodiments, the composition is administered to the subject in a dose and schedule sufficient to increase survival rate of the subject at least 5%. In some embodiments, the composition is administered to the subject in a dose and schedule sufficient to inhibit growth of the cancer.

### BRIEF DESCRIPTION OF THE DRAWINGS

FIG. 1 illustrates exemplary SOS1/2 mRNA knockdowns mediated by antisense oligonucleotides described herein.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Described herein are compositions and methods for modulating gene or signaling pathway expressions. Also described herein are compositions and methods for treating a disease or condition by modulating the gene or signaling pathway expression associated with the disease or condition. In some embodiments, the composition comprises at least one oligonucleotide that, upon being delivered into a cell, binds (e.g., hybridizes) to an endogenous mRNA, which leads to the degradation of the target mRNA. In some embodiments, described herein is a method for utilizing the composition or the oligonucleotide described herein. In some embodiments, the methods are used to treat the disease or condition by contacting a cell with the oligonucleotide to decrease the gene or signaling pathway expression associated with the disease or condition.

In some embodiments, the oligonucleotide is an antisense oligonucleotide, where the oligonucleotide is complementary and binds (e.g., hybridizes) to a segment of at least one endogenous nucleic acid (e.g., an mRNA). In some embodiments, the binding of the oligonucleotide to the endogenous nucleic acid leads to degradation of the endogenous nucleic acid or blocking of translation of the target protein from the endogenous nucleic acid, hence decrease of the expression of the gene encoded by the endogenous nucleic acid. For example, the binding of the oligonucleotide to the endogenous mRNA creates a duplex nucleic acid molecule, which can then recruit endogenous nuclease (e.g., RNaseH) for degradation of the mRNA.

In some embodiments, the oligonucleotide comprises a stretch of DNA nucleotides (such as a stretch of 5 to 10 DNA nucleotides) sufficient to recruit RNaseH when hybridized to the target mRNA. In embodiments, the stretch of DNA nucleotides is a gap segment. In some embodiments, the oligonucleotide comprises at least one wing segment. In some embodiments, the oligonucleotide comprises at least one gap segment flanked by two wing segments. For example, the oligonucleotide comprises a gap segment flanked by a 5′-wing segment and a 3′-wing segment. In some embodiments, the gap segment or the wing segment comprises at least one chemical modification. In some embodiments, the antisense oligonucleotide is a gapmer.

In some embodiments, the gene product (e.g., a transcript or a peptide/polypeptide transcribed or translated from the gene sequence of a gene, respectively) modulated by the oligonucleotide is part of the signaling pathway. In some embodiments, the signaling pathway is the RTK-SOS-RAS-ERK pathway. As such, in some embodiments, the decreasing of the expression of the gene due to the binding of the oligonucleotide to the endogenous nucleic acid can further decrease a signaling pathway expression comprising the gene product modulated by the oligonucleotide. In some embodiments, the decreasing gene or signaling pathway expression leads to therapeutic effects for treating the disease or condition. In some embodiments, the disease or condition is caused by increased gene or signaling pathway expression. In some embodiments, the disease or condition described herein is caused by genetic mutations associated with the gene or signaling pathway.

### Compositions

Described herein, in some embodiments, are compositions comprising at least one oligonucleotide described herein. In some embodiments, the composition comprises at least two, three, four, five, six, seven, eight, nine, ten, or more different oligonucleotides. In some embodiments, the oligonucleotide described herein is an antisense oligonucleotide for targeting and binding to an endogenous nucleic acid. In some embodiments, the binding of the oligonucleotide to the endogenous nucleic acid recruits endogenous nuclease for degrading the endogenous nucleic acid. In some embodiments, the binding of the oligonucleotide to the endogenous nucleic acid may inhibit the 5′cap formation, sterically hinder the recruitment of ribosome or ribosomal activity, altering the splicing of the endogenous nucleic acid. In some embodiments, any of the inhibition of the 5′cap formation, steric hinderance of the recruitment of the ribosome/ribosomal activity, alteration of the splicing, or the degradation of the endogenous nucleic acid decreases expression of the gene product encoded by the endogenous nucleic acid. In some embodiments, the degradation of the endogenous nucleic acid decreases expression of the gene product encoded by the endogenous nucleic acid. In some embodiments, the decreased expression of the gene product can treat a disease or condition described herein. In some embodiments, the degradation of the endogenous nucleic acid can treat a disease or condition described herein.

In some embodiments, the oligonucleotide comprises a length of at least five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some embodiments, the oligonucleotide comprises a length of at least 10, 11, 12, 13, 14, 15, 16, 17, 18, 19, or 20 nucleic acid bases. In some embodiments, the oligonucleotide comprises 10 nucleic acid bases. In some embodiments, the oligonucleotide comprises 11 nucleic acid bases. In some embodiments, the oligonucleotide comprises 12 nucleic acid bases. In some embodiments, the oligonucleotide comprises 13 nucleic acid bases. In some embodiments, the oligonucleotide comprises 14 nucleic acid bases. In some embodiments, the oligonucleotide comprises 15 nucleic acid bases. In some embodiments, the oligonucleotide comprises 16 nucleic acid bases. In some embodiments, the oligonucleotide comprises 17 nucleic acid bases. In some embodiments, the oligonucleotide comprises 18 nucleic acid bases. In some embodiments, the oligonucleotide comprises 19 nucleic acid bases. In some embodiments, the oligonucleotide comprises 20 nucleic acid bases.

In some embodiments, the oligonucleotide comprises at least one gap segment. In some embodiments, the gap segment comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some embodiments, the gap segment comprises four, five, six, seven, right, nine, 10, 11, 12, 13, or 14 nucleic acid bases. In some embodiments, the gap segment comprises four, five, or six nucleic acid bases. In some embodiments, the gap segment comprises five nucleic acid bases. In some embodiments, the gap segment comprises six nucleic acid bases. In some embodiments, the gap segment comprises seven nucleic acid bases. In some embodiments, the gap segment comprises eight nucleic acid bases. In some embodiments, the gap segment comprises nine nucleic acid bases. In some embodiments, the gap segment comprises 10 nucleic acid bases. In some embodiments, the gap segment comprises 11 nucleic acid bases. In some embodiments, the gap segment comprises 12 nucleic acid bases. In some embodiments, the gap segment comprises 13 nucleic acid bases. In some embodiments, the gap segment comprises 14 nucleic acid bases.

In some embodiments, the oligonucleotide comprises at least one wing segment. In some embodiments, the at least one wing segment is a 5′-end wing segment that is covalently connected to the gap segment at the 5′-end of the gap segment. In some embodiments, the at least one wing segment is a 3′-end wing segment that is covalently connected to the gap segment at the 3′-end of the gap segment. In some embodiments, the gap segment is flanked by the wing segments at both the 5′-end and the 3′-end of the gap segment. In some embodiments, the wing segment comprises at least one, two, three, four, five, six, seven, eight, nine, 10, 11, 12, or more nucleic acid bases. In some embodiments, the wing segment comprises one nucleic acid base. In some embodiments, the wing segment comprises two nucleic acid bases. In some embodiments, the wing segment comprises three nucleic acid bases. In some embodiments, the wing segment comprises four nucleic acid bases. In some embodiments, the wing segment comprises five nucleic acid bases. In some embodiments, the wing segment comprises six nucleic acid bases. In some embodiments, the wing segment comprises seven nucleic acid bases. In some embodiments, the wing segment comprises eight nucleic acid bases. In some embodiments, the wing segment comprises nine nucleic acid bases. In some embodiments, the wing segment comprises 10 nucleic acid bases. In embodiments, the length of the wing segments are independently selected from 2 to 5 nucleic acid bases.

In some embodiments, the oligonucleotide is an antisense oligonucleotide. In some embodiments, the antisense oligonucleotide binds to a target nucleic acid. In some embodiments, the oligonucleotide comprises a nucleic acid sequence that allows the oligonucleotide to bind to target nucleic acid by base pairing such as Watson Crick base pairing. Compositions and methods provided herein can be utilized to modulate expression of a gene or signaling pathway. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an oligonucleotide described herein can facilitate a knockdown. A knockdown can reduce the expression of a target RNA. In some cases, a knockdown can be accompanied by modulating of an mRNA. In some cases, a knockdown can occur with substantially little to no modulating of an mRNA. In some instances, a knockdown can occur by targeting an untranslated region of the target RNA, such as a 3′ UTR, a 5′ UTR or both. In some cases, a knockdown can occur by targeting a coding region of the target RNA.

In some embodiments, the oligonucleotide is an antisense oligonucleotide for targeting and binding any one of the gene products or nucleic acids described herein. In some embodiments, the gene product(s) being targeted and bound by the antisense oligonucleotide is SOS RAS/Rac guanine nucleotide exchange factor 1 or son of sevenless 1 (SOS1) and/or SOS RAS/Rac guanine nucleotide exchange factor 2 son of sevenless 2 (SOS2). In some embodiments, the gene product(s) being targeted and bound by the antisense oligonucleotide is SOS RAS/Rac guanine nucleotide exchange factor. In some embodiments, the antisense oligonucleotide targets and binds (i.e., specifically hybridizes to) to an mRNA of SOS1 (SEQ ID NOs: 190-192, Table 5) and/or mRNA of SOS2 (SEQ ID NO: 193, Table 5). In some embodiments, the antisense oligonucleotide targets and binds to an mRNA of SOS1, where the mRNA of SOS 1 is at least 70%, 75%, 80%, 85%, 90%, 95%, or 99% identical to SEQ ID NOs: 190-192. As used herein, the term “identity” includes the exchange of T nucleotides for U, and vice versa. In some embodiments, the antisense oligonucleotide targets and binds to an mRNA of SOS2 (SEQ ID NO: 193). In some embodiments, the antisense oligonucleotide targets and binds to an mRNA of SOS2, where the mRNA of SOS 2 is at least 70%, 75%, 80%, 85%, 90%, 95%, or 99% identical to SEQ ID NO: 193.

In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 563 to 622 of SEQ ID NO: 190; nucleic acid position 1074 to 1133 of SEQ ID NO: 191; nucleic acid position 1074 to 1133 of SEQ ID NO: 192; or nucleic acid position 740 to 799 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 569 to 588 of SEQ ID NO: 190; nucleic acid position 1080 to 1099 of SEQ ID NO: 191; nucleic acid position 1080 to 1099 of SEQ ID NO: 192; or nucleic acid position 746 to 765 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 1403 to 1462 of SEQ ID NO: 190; nucleic acid position 1914 to 1973 of SEQ ID NO: 191; nucleic acid position 1914 to 1973 of SEQ ID NO: 192; or nucleic acid position 1574 to 1633 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 1427 to 1449 of SEQ ID NO: 190; nucleic acid position 1938 to 1960 of SEQ ID NO: 191; nucleic acid position 1938 to 1960 of SEQ ID NO: 192; or nucleic acid position 1598 to 1620 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 1943 to 2062 of SEQ ID NO: 190, nucleic acid position 2454 to 2573 of SEQ ID NO: 191; nucleic acid position 2454 to 2573 of SEQ ID NO: 192; or nucleic acid position 2114 to 2233 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 1973 to 2004 of SEQ ID NO: 190, nucleic acid position 2484 to 2515 of SEQ ID NO: 191; nucleic acid position 2484 to 2515 of SEQ ID NO: 192; or nucleic acid position 2144 to 2175 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 3143 to 3202 of SEQ ID NO: 190, nucleic acid position 3654 to 3713 of SEQ ID NO: 191; nucleic acid position 3654 to 3713 of SEQ ID NO: 192; or nucleic acid position 3314 to 3373 of SEQ ID NO: 193. In some embodiments, the antisense oligonucleotide targets and binds to a nucleic acid fragment of an mRNA of SOS1 or SOS2, where the nucleic acid fragment comprises a nucleic acid sequence that is identical to a nucleic acid sequence between: nucleic acid position 3158 to 3178 of SEQ ID NO: 190, nucleic acid position 3669 to 3689 of SEQ ID NO: 191; nucleic acid position 3669 to 3689 of SEQ ID NO: 192; or nucleic acid position 3328 to 3348 of SEQ ID NO: 193. In some embodiments, the antisense oligonucleotide targeting and binding to the nucleic acid fragment between any one the nucleic acid position corresponding to any one of SEQ ID NOS: 190-193 described herein comprises at least one wobble nucleic acid (e.g., the antisense oligonucleotide can comprise at least one mismatch with the nucleic acid fragment of the SOS1 or SOS2 mRNA). In some embodiments, the antisense oligonucleotide targeting and binding to the nucleic acid fragment between any one the nucleic acid position corresponding to any one of SEQ ID NOS: 190-193 described herein comprises one wobble nucleic acid (e.g., the antisense oligonucleotide can comprise one mismatch with the nucleic acid fragment of the SOS1 or SOS2 mRNA). In some embodiments, the antisense oligonucleotide targeting and binding to the nucleic acid fragment between any one the nucleic acid position corresponding to any one of SEQ ID NOS: 190-193 described herein comprises at least two wobble nucleic acids (e.g., the antisense oligonucleotide can comprise at least two mismatches with the nucleic acid fragment of the SOS1 or SOS2 mRNA). In some embodiments, the antisense oligonucleotide targeting and binding to the nucleic acid fragment between any one the nucleic acid position corresponding to any one of SEQ ID NOS: 190-193 described herein comprises two wobble nucleic acids (e.g., the antisense oligonucleotide can comprise two mismatches with the nucleic acid fragment of the SOS1 or SOS2 mRNA). In some embodiments, the antisense oligonucleotide targeting and binding to the nucleic acid fragment between any one the nucleic acid position corresponding to any one of SEQ ID NOS: 190-193 comprises at least five, at least six, at least seven, at least nine, at least 10, at least 11, or at least 12 consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises five consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises six consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises seven consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises eight consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises nine consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises 10 consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises 11 consecutive nucleotides that are identical to the nucleic acid fragment. In some embodiments, the antisense oligonucleotide targeting and binding to the nucleic acid fragment between any one the nucleic acid position corresponding to any one of SEQ ID NOS: 190-193 comprises 12 consecutive nucleotides that are identical to the nucleic acid fragment.

In some embodiments, the antisense oligonucleotide comprises a nucleic acid sequence that is at least 70%, 75%, 80%, 85%, 90%, 95%, or 99% identical (or 100% identical) to any one of SEQ ID NOs: 1-189. In some embodiments, the antisense oligonucleotide described herein can target and bind to either or both SOS1 mRNA or SOS2 mRNA. In some embodiments, the antisense oligonucleotide comprises at least one gap segment. In some embodiments, the at least one gap segment comprises a nucleic acid sequence that is at least 70%, 75%, 80%, 85%, 90%, 95%, or 99% identical (or 100% identical) to any one of SEQ ID NOs: 1-189.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical (or 100% identical) to any one of SEQ ID NOs: 1-189. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of any one of SEQ ID NOs: 1-189.

In some embodiments, the oligonucleotide comprises at least one gap segment. In some embodiments, the at least one gap segment comprises an nucleic acid sequence that is at least 70%, 75%, 80%, 85%, 90%, 95%, or 99% identical (or 100% identical) to any one of SEQ ID NOs: 1-189. In some embodiments, the at least one gap segment comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of any one of SEQ ID NOs: 1-189.

In some embodiments, the antisense oligonucleotide comprises a nucleic acid sequence that is at least 70%, 75%, 80%, 85%, 90%, 95%, or 99% identical (or 100% identical) to any one of SEQ ID NOs: 2-189 (Table 4). In some embodiments, the antisense oligonucleotide described herein can target and bind to either or both SOS1 mRNA or SOS2 mRNA. In some embodiments, the antisense oligonucleotide comprises at least one gap segment. In some embodiments, the at least one gap segment comprises a nucleic acid sequence that is at least 70%, 75%, 80%, 85%, 90%, 95%, or 99% identical (or 100% identical) to any one of SEQ ID Nos: 2-189 (Table 4).

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical (or 100% identical) to any one of SEQ ID NOs: 2-189 (Table 4). In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of any one of SEQ ID NOs: 2-189 (Table 4).

In some embodiments, the antisense oligonucleotide comprises or consists of a nucleic acid sequence of SEQ ID NOs: 1, 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 or 187. In some embodiments, the antisense oligonucleotide described herein can target and bind to either or both SOS1 mRNA or SOS2 mRNA. In some embodiments, the antisense oligonucleotide comprises at least one gap segment. In some embodiments, the antisense oligonucleotide has a structure (i.e., nucleobase sequence and chemical modification pattern) shown in Table 4.

In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of any one of SEQ ID NOs: 1, 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 or 187.

In some embodiments, the antisense oligonucleotide comprises a nucleic acid sequence that is at least 70%, 75%, 80%, 85%, 90%, 95%, or 99% identical (or 100% identical) to any one of SEQ ID NOs: 20, 31, 42, 56, 65, 66, 73, 94, 95, 101, 107, 139, 158, or 187. In some embodiments, the antisense oligonucleotide described herein can target and bind to either or both SOS1 mRNA or SOS2 mRNA (i.e., is complementary to an equal length segment of the target mRNA). In some embodiments, the antisense oligonucleotide comprises at least one gap segment. In some embodiments, the at least one gap segment comprises a nucleic acid sequence that is at least 70%, 75%, 80%, 85%, 90%, 95%, or 99% identical (or 100% identical) to any one of SEQ ID NOs: 20, 31, 42, 56, 65, 66, 73, 94, 95, 101, 107, 139, 158, or 187. In embodiments, the antisense oligonucleotide comprises or consists of a nucleotide sequence selected from SEQ ID NOS: 187, 20, 31, 42, 56, 65, 66, 73, 94, 95, 101, 107, 139, and 158.

In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of any one of SEQ ID NOs: 20, 31, 42, 56, 65, 66, 73, 94, 95, 101, 107, 139, 158, or 187.

In some embodiments, the antisense oligonucleotide comprises a nucleic acid sequence that comprises or consist of the nucleic acid sequence of SEQ ID NO: 20. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 20. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 20.

In some embodiments, the antisense oligonucleotide comprises a nucleic acid sequence that comprises or consists of the nucleic acid sequence of SEQ ID NO: 31. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 31. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 31.

In some embodiments, the antisense oligonucleotide comprises a nucleic acid sequence that comprises or consists of the nucleic acid sequence of SEQ ID NO: 42. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 42. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 42.

In some embodiments, the antisense oligonucleotide comprises a nucleic acid sequence that comprises or consists of the nucleic acid sequence of SEQ ID NO: 56. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 56. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 56.

In some embodiments, the antisense oligonucleotide comprises a nucleic acid sequence that comprises or consists of the nucleic acid sequence of SEQ ID NO: 65. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 65. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 65.

In some embodiments, the antisense oligonucleotide comprises a nucleic acid sequence that comprises or consists of the nucleic acid sequence of SEQ ID NO: 66. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 66. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 66.

In some embodiments, the antisense oligonucleotide comprises a nucleic acid sequence that comprises or consists of the nucleic acid sequence of SEQ ID NO: 73. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 73. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 73.

In some embodiments, the antisense oligonucleotide comprises a nucleic acid sequence that comprises or consists of the nucleic acid sequence of SEQ ID NO: 94. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 94. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 94.

In some embodiments, the antisense oligonucleotide comprises a nucleic acid sequence that comprises or consists of the nucleic acid sequence of SEQ ID NO: 95. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 95. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 95.

In some embodiments, the antisense oligonucleotide comprises a nucleic acid sequence that comprises or consists of the nucleic acid sequence of SEQ ID NO: 101. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 101. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 101.

In some embodiments, the antisense oligonucleotide comprises a nucleic acid sequence that comprises or consists of the nucleic acid sequence of SEQ ID NO: 107. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 107. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 107.

In some embodiments, the antisense oligonucleotide comprises a nucleic acid sequence that comprises or consists of the nucleotide sequence of SEQ ID NO: 139. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 139. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 139.

In some embodiments, the antisense oligonucleotide comprises a nucleic acid sequence that comprises or consists of SEQ ID NO: 158. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 158. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 158.

In some embodiments, the antisense oligonucleotide comprises a nucleic acid sequence that comprises or consists of the nucleic acid sequence of SEQ ID NO: 187. In some embodiments, the antisense oligonucleotide described herein can target and bind to either or both SOS1 mRNA or SOS2 mRNA. In some embodiments, the antisense oligonucleotide comprises at least one gap segment.

In some embodiments, the antisense oligonucleotide targets and binds to an mRNA of SOS1 and/or SOS2. In some embodiments, the antisense oligonucleotide comprises an nucleic acid sequence that is at least 70%, 75%, 80%, 85%, 90%, 95%, or 99% identical to SEQ ID NO: 187. In some embodiments, the antisense oligonucleotide comprises a nucleic acid sequence comprising at least three, at least four, at least five, at least six, at least seven, at least eight, at least nine, at least 10, at least 11, at least 12, at least 13, at least 14, at least 15, or more consecutive nucleotides of a nucleic acid sequence of SEQ ID NO: 187.

In some embodiments, the binding (e.g., hybridization) of the antisense oligonucleotide to the target mRNA leads to degradation of the target mRNA or blocks translation of the target mRNA. In some embodiments, the binding of the antisense oligonucleotide to the target mRNA creates a duplex nucleic acid molecule, which then recruits an endogenous nuclease for degradation of the mRNA. In some embodiments, the antisense oligonucleotide has a stretch of DNA nucleotides sufficient to recruit RNaseH, and thereby trigger degradation of the target mRNA. For example, the antisense oligonucleotide may have a stretch (e.g., a central stretch) of at least 6 or at least 8 DNA nucleotides (e.g., 6, 7, or 8 nucleotides). In some embodiments, one or more DNA nucleotides comprise a 2′ chemical modification independently selected from 2′-Fluoro, 2′-Methyl, and 2′-Ethyl. In some embodiments, DNA nucleotides contain a 2′-H. For example, the antisense oligonucleotide may be a gapmer having a 5′ and a 3′ segment, each of the 5′ and 3′ segments being (independently selected) from 2 to 6 nucleotides or from 2 to 5 nucleotides (e.g., 3, 4, or 5 nucleotides), and where the 5′ and 3′ segments do not contain DNA nucleotides. In some embodiments, the gapmer is a 3-8-3 gapmer, having a central bock of DNA nucleotides and 5′ and 3′ segments of 3 RNA nucleotides each (which are optionally modified RNA nucleotides). In some embodiments, the antisense oligonucleotide is a 4-8-3 gapmer, a 3-8-4 gapmer, or a 4-8-4 gapmer. In some embodiments, one or more nucleotides of the 5′ segment and the 3′ segment comprise 2′-O substituents, optionally where all of the nucleotides of the 5′ segment and the 3′ segment comprise 2′-O substituents. Exemplary 2′-O substituents are independently selected from 2′-O methyl, 2′-O ethyl, 2′-O methoxyethyl (MOE), and a bridged nucleotide (e.g., a locked or bi-cyclic nucleotide) having a 2′ to 4′ bridge. In some embodiments, the bridged nucleotide has a methylene bridge (LNA) or a constrained ethyl bridge (cEt), or other bridged nucleotide described herein.

The term “gapmer” refers to an oligonucleotide having a central block of deoxynucleotides (also referred to herein as “DNA nucleotides”) with 5′ and 3′ segments (of at least 2 nucleotides) of RNA nucleotides. As used herein, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl.

Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge and a constrained ethyl (cEt) bridge (see, U.S. Pat. Nos. 7,399,845 and 7,569,686, which are hereby incorporated by reference in their entireties).

For simplicity nucleotide sequences may be shown herein using DNA nucleotide sequences (i.e., including T nucleobases) or as RNA nucleotide sequences (i.e., including U nucleobases). It is understood from the context that when a nucleotide or sequence is intended to be RNA, T nucleotides are substituted with U (or modified U such as pseudouridine or 1-methylpseudouridine); and when the nucleotide or sequence is intended to be DNA, T nucleotides are employed. However, in embodiments, RNA nucleotides in the antisense oligonucleotide may employ T (thymine) bases, and DNA nucleotides in the antisense oligonucleotides may employ U (uracil) bases.

In embodiments, antisense oligonucleotides can be fully phosphorothioate linked, as described elsewhere herein.

In some embodiments, the antisense oligonucleotide may contain one or more modified bases, as described elsewhere herein. In some embodiments, cytosine is replaced with 5-methylcytosine, which may enhance base pairing. Other modified bases (particularly of cytosine or guanine) can be employed to reduce immunogenicity, where needed. Other modified bases are described in U.S. Pat. No. 10,064,959, which is hereby incorporated by reference. In various embodiments, cytidine nucleobases in the antisense oligonucleotide are 5-methyl cytidine. Thus, it will be understood by the skilled person that where a sequence includes a cytidine nucleobase (“C”), the term includes 5-methyl C. Further, it is understood that where uracil bases are included in a sequence, the term “U” includes pseudouridine and N1-methylpseudouridine.

In some embodiments, the oligonucleotide described herein targets and binds an endogenous nucleic acid encoding a gene product associated with the KRAS-RAF-MEK-ERK signaling pathway. In some embodiments, the gene product associated with the KRAS-RAF-MEK-ERK signaling pathway is SOS1. In some embodiments, the gene product associated with the KRAS-RAF-MEK-ERK signaling pathway is SOS2. In some embodiments, the oligonucleotide described herein modulates or affects the expression or activity of a gene product in or associated with the KRAS-RAF-MEK-ERK signaling pathway. In some embodiments, the gene product associated with the KRAS-RAF-MEK-ERK signaling pathway is RAS. In some embodiments, the gene product associated with the KRAS-RAF-MEK-ERK signaling pathway is RAF. In some embodiments, the gene product associated with the KRAS-RAF-MEK-ERK signaling pathway is MEK. In some embodiments, the gene product associated with the KRAS-RAF-MEK-ERK signaling pathway is ERK.

In some embodiments, the oligonucleotide, upon binding to the endogenous nucleic acid, forms a duplex with the endogenous nucleic acid and recruits an endogenous nuclease for degrading the endogenous nucleic acid. In some embodiments, the endogenous nuclease is a deoxyribonuclease. In some embodiments, the endogenous nuclease is a ribonuclease. In some embodiments, the ribonucleases is an endoribonuclease. In some embodiments, the endoribonuclease comprises endoribonuclease or RNase A, P, H, I, III, T1, T2, U2, V1, PhyM, or V. In some embodiments, the ribonuclease is an exoribonuclease. In some embodiments, the exoribonuclease comprises RNase P, H, II, R, D, or T. In some embodiments, the nuclease comprises polynucleotide phosphorylase (PNPase), oligoribonuclease, exoribonuclease I, or exoribonuclease II. In some embodiments, the ribonuclease recruited by the oligonucleotide binding to the endogenous nucleic acid is RNase H.

In some embodiments, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some embodiments, the oligonucleotide comprises at least one gap segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some embodiments, the oligonucleotide comprises at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some embodiments, the oligonucleotide comprises at least one gap segment and at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications.

In some embodiments, the oligonucleotide described herein binds to an endogenous nucleic acid (e.g., an mRNA) encoding SOS1, where the binding of the oligonucleotide to the SOS1 endogenous nucleic acid decreases the endogenous expression of SOS1 in a cell by at least 10%, 20%, 30%, 40%, 50%, 60%, 70%, 80%, 90%, or more compared to the endogenous expression of SOS1 not modulated by the oligonucleotide. In some embodiments, the oligonucleotide described herein binds to an endogenous nucleic acid (e.g., an mRNA) encoding SOS2, where the binding of the oligonucleotide to the SOS2 endogenous nucleic acid decreases the endogenous expression of SOS2 in a cell by at least 10%, 20%, 30%, 40%, 50%, 60%, 70%, 80%, 90%, or more compared to the endogenous expression of SOS2 not modulated by the oligonucleotide.

In some embodiments, the oligonucleotide described herein binds to an endogenous nucleic acid (e.g., an mRNA) encoding SOS1, where the binding of the oligonucleotide to the SOS1 endogenous nucleic acid decreases the endogenous expression of a gene in, or an activity of, the KRAS-RAF-MEK-ERK signaling pathway in a cell by at least 10%, 20%, 30%, 40%, 50%, 60%, 70%, 80%, 90%, or more compared to the endogenous expression of the KRAS-RAF-MEK-ERK signaling pathway not modulated by the oligonucleotide.

In some embodiments, the oligonucleotide described herein binds to an endogenous nucleic acid (e.g., an mRNA) encoding SOS2, where the binding of the oligonucleotide to the SOS2 endogenous nucleic acid decreases the endogenous expression of a gene in, or an activity of, the KRAS-RAF-MEK-ERK signaling pathway in a cell by at least 10%, 20%, 30%, 40%, 50%, 60%, 70%, 80%, 90%, or more compared to the endogenous expression of a gene in, or an activity of the KRAS-RAF-MEK-ERK signaling pathway not modulated by the oligonucleotide.

In some embodiments, the composition comprises at least two oligonucleotides, where a first oligonucleotide binds to the SOS1 endogenous nucleic acid (e.g., an SOS1 mRNA) and a second oligonucleotide binds to the SOS2 endogenous nucleic acid (e.g., an SOS2 mRNA). In some embodiments, the binding of the oligonucleotide to both SOS1 and SOS2 endogenous nucleic acids decreases the endogenous expression of a gene in, or an activity of, the KRAS-RAF-MEK-ERK signaling pathway in a cell by at least 10%, 20%, 30%, 40%, 50%, 60%, 70%, 80%, 90%, or more compared to the endogenous expression of a gene in, or an activity of the KRAS-RAF-MEK-ERK signaling pathway not modulated by the oligonucleotide. In some embodiments, the binding of the oligonucleotide to both SOS1 and SOS2 endogenous nucleic acids increases killing of cancer cell by at least 10%, 20%, 30%, 40%, 50%, 60%, 70%, 80%, 90%, or more compared to binding of an oligonucleotide to either SOS1 or SOS2 only. In some embodiments, the binding of the oligonucleotide to both SOS1 and SOS2 endogenous nucleic acids decreases cellular proliferation by at least 10%, 20%, 30%, 40%, 50%, 60%, 70%, 80%, 90%, or more compared to binding of an oligonucleotide to either SOS1 or SOS2 only.

In some embodiments, the composition is formulated for administration to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some embodiments, the composition is formulated into a dosage form. In some embodiments, the composition is formulated to include at least one excipient. In some embodiments, the excipient is a pharmaceutically acceptable excipient.

In some embodiments, the composition comprising the oligonucleotide described herein is used to treat a disease or condition by decreasing the endogenous expression of a gene in, or an activity of, the KRAS-RAF-MEK-ERK signaling pathway associated with the disease or condition. In some embodiments, the composition comprising the oligonucleotide describe herein treats the disease or condition described herein by directly decreasing the gene expression associated with disease or condition described herein. In some embodiments, the composition comprising the oligonucleotide treats the disease or condition by decreasing the gene expression as part of a signaling pathway described herein. In some embodiments, the composition comprising the oligonucleotide described herein treats a disease or condition by decreasing the endogenous SOS1 expression. In some embodiments, the composition comprising the oligonucleotide described herein treats a disease or condition by decreasing the endogenous SOS2 expression. In some embodiments, the composition comprising the oligonucleotide described herein treats a disease or condition by decreasing both endogenous SOS1 and SOS2 expressions. In some embodiments, the composition comprising the oligonucleotide described herein treats a disease or condition by decreasing the endogenous KRAS expression. In some embodiments, the composition comprising the oligonucleotide described herein treats a disease or condition by decreasing the endogenous KRAS-RAF-MEK-ERK signaling pathway expression or activity. In some embodiments, the disease or condition described herein is cancer.

### Chemical Modifications

Described herein, in some embodiments, is an oligonucleotide comprising at least one chemical modification. In some embodiments, the oligonucleotide is single-stranded. In some embodiments, the oligonucleotide is an antisense oligonucleotide. In some embodiments, the oligonucleotide comprises ribonucleotides, deoxyribonucleotides, chemically-modified derivative thereof, or a combination thereof. In some embodiments, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more chemical modifications. In some embodiments, the oligonucleotide does not have an intramolecular structure feature. In some embodiments, the oligonucleotide comprises at least one gap segment comprising at least one, two, three, four, five, six, seven, eight, nine, ten, 11, 12, 13, 14, 15, or more chemically modified nucleotides. In some embodiments, the oligonucleotide comprises at least one wing segment comprising at least one, two, three, four, five, six, seven, eight, nine, ten, or more chemically modified nucleotides. In some embodiments, the oligonucleotide comprises a 5′-end wing segment comprising at least one, two, three, four, five, six, seven, eight, nine, ten, or more chemically modified nucleotides. In some embodiments, the oligonucleotide comprises a 3′-end wing segment comprising at least one, two, three, four, five, six, seven, eight, nine, ten, or more chemically modified nucleotides. In some embodiments, the at least one wing segment is covalently fused to the 5′-end of the gap segment. In some embodiments, the at least one wing segment is covalently fused to the 3′-end of the gap segment.

In some embodiments, the oligonucleotide comprises at least one, two, three, four, five, six, seven, eight, nine, ten, 11, 12, 13, 14, 15, 16, 17, 18, 19, 20 or more chemically modified nucleotides at the 5′ end of the oligonucleotide. In some embodiments, the oligonucleotide comprises at least one, two, three, four, five, six, seven, eight, nine, ten, 11, 12, 13, 14, 15, 16, 17, 18, 19, 20 or more chemically modified nucleotides at the 3′ end of the oligonucleotide. In some embodiments, the oligonucleotide comprises at least one, two, three, four, five, six, seven, eight, nine, ten, 11, 12, 13, 14, 15, 16, 17, 18, 19, 20 or more chemically modified nucleotides at both the 5′ and the 3′ end of the oligonucleotide. In some embodiments, the oligonucleotide comprises at least one chemical modification in the gap segment of the oligonucleotide. In some embodiments, the oligonucleotide comprises at least one chemical modification in the nucleotide bases adjacent the gap segment. In some embodiments, at least 10%, 20%, 30%, 40%, 50%, 60%, 70%, 80%, 90% of the bases or internucleotide linkage of the oligonucleotide comprises modifications. In some embodiments, the oligonucleotide comprises 100% modified nucleotide bases.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the oligonucleotide comprises at least one chemically modified base. In some instances, the comprises at least one, two, three, four, five, six, seven, eight, nine, 10, 15, 20, or more modified bases. In some cases, chemical modifications to the base moiety include natural and synthetic modifications of adenine, guanine, cytosine, thymine, or uracil, and purine or pyrimidine bases.

In some embodiments, the at least one chemical modification of the oligonucleotide comprises a modification of any one of or any combination of: 2′ modified nucleotide comprising 2′-O-methyl, 2′-O-methoxyethyl(2′-O-MOE), 2′-O-aminopropyl, 2′-deoxy, 2′-deoxy-2′-fluoro, 2′-O-aminopropyl(2′-O-AP), 2′-O-dimethylaminoethyl(2′-O-DMAOE), 2′-O-dimethylaminopropyl (2′-O-DMAP), 2′-O-dimethylaminoethyloxyethyl(2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of chemical modification to the oligonucleotide can include: modification of one or both of non-linking or linking phosphate oxygens in the phosphodiester backbone linkage (e.g., sulfur(S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap-OH) or 3′ end of the nucleic acid sequence (3′ tail or modification of 3′ end-OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl(2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some embodiments, the chemical modification of the oligonucleotide comprises at least one substitution of one or both of non-linking phosphate oxygen atoms in a phosphodiester backbone linkage of the oligonucleotide. In some embodiments, the at least one chemical modification of the oligonucleotide comprises a substitution of one or more of linking phosphate oxygen atoms in a phosphodiester backbone linkage of the oligonucleotide. A non-limiting example of a chemical modification of a phosphate oxygen atom is a sulfur atom. In some embodiments, the chemical modification of the oligonucleotide comprises at least one chemical modification to a sugar of a nucleotide of the oligonucleotide. In some embodiments, the chemical modification of the oligonucleotide comprises at least one chemical modification to the sugar of the nucleotide, where the chemical modification comprises at least one locked nucleic acid (LNA). In some embodiments, the chemical modification of the oligonucleotide comprises at least one chemical modification to the sugar of the nucleotide of the oligonucleotide comprising at least one unlocked nucleic acid (UNA). In some embodiments, the chemical modification of the oligonucleotide comprises at least one chemical modification to the sugar of the nucleotide of the oligonucleotide comprising at least one ethylene nucleic acid (ENA). In some embodiments, the chemical modification of the oligonucleotide comprises at least one chemical modification to the sugar comprising a modification of a constituent of the sugar, where the sugar is a ribose sugar. In some embodiments, the chemical modification of the oligonucleotide comprises at least one chemical modification to the constituent of the ribose sugar of the nucleotide of the oligonucleotide comprising a 2′-O-methyl group. In some embodiments, the chemical modification of the oligonucleotide comprises at least one chemical modification comprising replacement of a phosphate moiety of the oligonucleotide with a dephospho linker. In some embodiments, the chemical modification of the oligonucleotide comprises at least one chemical modification of a phosphate backbone of the oligonucleotide. In some embodiments, the oligonucleotide comprises a phosphothioate group. In some embodiments, the chemical modification of the oligonucleotide comprises at least one chemical modification comprising a modification to a base of a nucleotide of the oligonucleotide. In some embodiments, the chemical modification of the oligonucleotide comprises at least one chemical modification comprising an unnatural base of a nucleotide. In some embodiments, the chemical modification of the oligonucleotide comprises at least one chemical modification comprising a morpholino group (e.g., a phosphorodiamidate morpholino oligomer, PMO), a cyclobutyl group, pyrrolidine group, or peptide nucleic acid (PNA) nucleoside surrogate. In some embodiments, the chemical modification of the oligonucleotide comprises at least one chemical modification comprising at least one stereopure nucleic acid. In some embodiments, the at least one chemical modification can be positioned proximal to a 5′ end of the oligonucleotide. In some embodiments, the at least one chemical modification can be positioned proximal to a 3′ end of the oligonucleotide. In some embodiments, the at least one chemical modification can be positioned proximal to both 5′ and 3′ ends of the oligonucleotide.

In some embodiments, an oligonucleotide comprises a backbone comprising a plurality of sugar and phosphate moieties covalently linked together. In some cases, a backbone of an oligonucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an oligonucleotide can lack a 5′ reducing hydroxyl, a 3′ reducing hydroxyl, or both, capable of being exposed to a solvent. In some embodiments, a backbone of an oligonucleotide can lack a 5′ reducing hydroxyl, a 3′ reducing hydroxyl, or both, capable of being exposed to nucleases. In some embodiments, a backbone of an oligonucleotide can lack a 5′ reducing hydroxyl, a 3′ reducing hydroxyl, or both, capable of being exposed to hydrolytic enzymes. In some instances, a backbone of an oligonucleotide can be represented as a polynucleotide sequence in a circular 2-dimensional format with one nucleotide after the other. In some instances, a backbone of an oligonucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some embodiments, the oligonucleotide described herein comprises at least one chemical modification. A chemical modification can be a substitution, insertion, deletion, chemical modification, physical modification, stabilization, purification, or any combination thereof. In some cases, a modification is a chemical modification. Suitable chemical modifications comprise any one of: 5′ adenylate, 5′ guanosine-triphosphate cap, 5′ N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2-O-methyl 3phosphorothioate or any combinations thereof.

In some cases, a modification can be permanent. In other cases, a modification can be transient. In some cases, multiple modifications are made to the oligonucleotide. the oligonucleotide modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some instances, chemical modifications to enhance guide stability, synthesis, localization, intracellular retention, or lengthen half-lives may not be genetically encodable. An oligonucleotide can be circular, substantially circular, or otherwise linked in a contiguous fashion (e.g., can be arranged as a loop) and can also retain a substantially similar secondary structure as a substantially similar oligonucleotide that may not be circular or may not be a loop.

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some embodiments, the chiral phosphate product is present in a diastereomeric excess of 50%, 60%, 70%, 80%, 90%, or more. In some embodiments, the chiral phosphate product is present in a diastereomeric excess of 95%. In some embodiments, the chiral phosphate product is present in a diastereomeric excess of 96%. In some embodiments, the chiral phosphate product is present in a diastereomeric excess of 97%. In some embodiments, the chiral phosphate product is present in a diastereomeric excess of 98%. In some embodiments, the chiral phosphate product is present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g., a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the oligonucleotide described herein comprises at least one chemically modified phosphate backbone. Exemplary chemically modification of the phosphate group or backbone can include replacing one or more of the oxygens with a different substituent. Furthermore, the modified nucleotide present in the oligonucleotide can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified oligonucleotide can be stereopure (e.g., S or R confirmation). In some cases, the chemically modified oligonucleotide comprises stereopure phosphate modification. For example, the chemically modified oligonucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the oligonucleotide can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the oligonucleotide described herein comprises at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein comprises modifying ribophosphate backbone of the oligonucleotide. In some embodiments, the oligonucleotide described herein comprises at least one chemically modified ribophosphate backbone. Exemplary chemically modified ribophosphate backbone can include scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include morpholino such as a phosphorodiamidate morpholino oligomer (PMO), cyclobutyl, pyrrolidine and peptide nucleic acid (PNA) nucleoside surrogates.

### Modification of Sugar

In some embodiments, the chemical modification described herein comprises modification of sugar. In some embodiments, the oligonucleotide described herein comprises at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the I position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g., L-nucleosides. In some aspects, the oligonucleotide described herein includes the sugar group ribose, which is a 5-membered ring having an oxygen. Exemplary modified nucleosides and modified nucleotides can include replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some embodiments, the modifications to the sugar of the oligonucleotide comprises modifying the oligonucleotide to include locked nucleic acid (LNA), unlocked nucleic acid (UNA), ethylene nucleic acid (ENA), constrained ethyl (cEt) sugar, or bridged nucleic acid (BNA).

### Modification of a Constituent of the Ribose Sugar

In some embodiments, the oligonucleotide described herein comprises at least one chemical modification of a constituent of the ribose sugar. In some embodiments, the chemical modification of the constituent of the ribose sugar can include 2′-O-methyl, 2′-O-methoxyethyl(2′-O-MOE), 2′-fluoro, 2′-aminoethyl, 2′-deoxy-2′-fuloarabinou-cleic acid, 2′-deoxy, 2′-deoxy-2′-fluoro, 2′-O-methyl, 3′-phosphorothioate, 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), 2′-O—N-methylacetamido (2′-O-NMA) 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂(R═H, C₁-C₁₂ alkyl or a protecting group); and combinations thereof.

In some instances, the oligonucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl, 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′—CH(CH₃)—O-2′ and 4′-CH(CH₂OCH₃)—O-2′, and analogs thereof; 4′-C(CH₃) (CH₃)—O-2′ and analogs thereof.

### Modifications on the Base of Nucleotide

In some embodiments, the chemical modification described herein comprises modification of the base of nucleotide (e.g., the nucleobase). Exemplary nucleobases can include adenine (A), thymine (T), guanine (G), cytosine (C), and uracil (U). These nucleobases can be modified or replaced to in the oligonucleotide described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein comprises modifying an uracil. In some embodiments, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein comprises modifying a cytosine. In some embodiments, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar (p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, 06-Methyl-2′-deoxyguanosine, 2′-F-ara-guanosine, and 2′-F-guanosine.

In some cases, the chemical modification of the oligonucleotide can include introducing or substituting a nucleic acid analog or an unnatural nucleic acid into the oligonucleotide. In some embodiments, nucleic acid analog can be any one of the chemically modified nucleic acid described herein. all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxy guanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-ami nohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-tauri nomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-th io-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), G-clamps, phenoxazine cytidine (e.g., 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification comprises chemically modifying the 5′ or 3′ end such as 5′ cap or 3′ tail of the oligonucleotide. In some embodiments, the oligonucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the oligonucleotide. In some embodiments, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the oligonucleotide can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

In some embodiments, the oligonucleotide comprising at least one chemical modification, upon binding to the target RNA, is more specific in recruiting the endogenous nuclease for decreasing expression the target RNA compared to an oligonucleotide sharing identical nucleic acid sequence, but without any chemical modification, with the oligonucleotide comprising at least one chemical modification. In some embodiments, the oligonucleotide comprising at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more specific in recruiting the endogenous nuclease for decreasing expression the target RNA compared to an oligonucleotide sharing identical nucleic acid sequence, but without any chemical modification, with the oligonucleotide comprising at least one chemical modification.

In some embodiments, the oligonucleotide comprising at least one chemical modification comprises an increased resistance towards degradation by hydrolysis or nuclease digestion compared to an oligonucleotide sharing identical nucleic acid sequence, but without any chemical modification, with the oligonucleotide comprising at least one chemical modification. In some embodiments, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis compared to an oligonucleotide sharing identical nucleic acid sequence, but without any chemical modification, with the oligonucleotide comprising at least one chemical modification.

In some embodiments, the oligonucleotide comprising at least one chemical modification induces less immunogenicity or less innate immune response compared an oligonucleotide sharing identical nucleic acid sequence.

In some embodiments, the oligonucleotide comprising at least one chemical modification, when contacted with the target RNA, is less likely to induce off-target modulating of the target RNA compared to the off-target modulating of the target RNA induced by an oligonucleotide sharing identical nucleic acid sequence, but without any chemical modification, with the oligonucleotide comprising at least one chemical modification. In some embodiments, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce off-target modulating compared to off-target modulating induced by an oligonucleotide sharing identical nucleic acid sequence, but without any chemical modification, with the oligonucleotide comprising at least one chemical modification.

### Methods of Delivery

Described herein, in some embodiments, are methods of delivering the oligonucleotide described herein to a cell. In some embodiments, the method comprises delivering directly or indirectly an oligonucleotide to the cell. In some embodiments, the method comprises contacting the cell with the composition or the oligonucleotide described herein. In some embodiments, the method comprises expressing the composition or the oligonucleotide described herein in the cell. In some embodiments, the oligonucleotide or vector encoding the oligonucleotide can be delivered into the cell via any of the transfection methods described herein. In some embodiments, the oligonucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the oligonucleotide or vector encoding the oligonucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of oligonucleotide or vector encoding the oligonucleotide into a host cell is calcium phosphate transfection.

Chemical means for introducing the oligonucleotide or vector encoding the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide or vector encoding the oligonucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo or in vivo). In another aspect, the oligonucleotide or vector encoding the oligonucleotide can be associated with a lipid. The oligonucleotide or vector encoding the oligonucleotide associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the oligonucleotide or vector encoding the oligonucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the oligonucleotide or vector encoding the oligonucleotide described herein can be administered to the subject in need thereof via the use of the transgenic cells generated by introduction of the oligonucleotide or vector encoding the oligonucleotide first into allogeneic or autologous cells. In some cases, the cell can be isolated. In some embodiments, the cell can be isolated from the subject.

In some embodiments, the oligonucleotide described herein is conjugated. In some embodiments, the oligonucleotide is conjugated to with a peptide, antibody, lipid, carbohydrate, or polymer. In some embodiments, the oligonucleotide is conjugated to with a peptide, antibody, lipid, carbohydrate, or polymer at the 5′ end of the oligonucleotide. In some embodiments, the oligonucleotide is conjugated to with a peptide, antibody, lipid, carbohydrate, or polymer at the 3′ end of the oligonucleotide. In some embodiments, the oligonucleotide is conjugated to with a peptide, antibody, lipid, carbohydrate, or polymer at any nucleic acid residue of the oligonucleotide. In some embodiments, the peptide, antibody, lipid, carbohydrate, or polymer conjugated to the oligonucleotide confers therapeutic effect. For example, the peptide, antibody, lipid, carbohydrate, or polymer conjugated to the oligonucleotide can be cytotoxic drug or drug for treating cancer. In some embodiments, the peptide, antibody, lipid, carbohydrate, or polymer conjugated to the oligonucleotide increases the efficiency of the oligonucleotide binding to the endogenous nucleic acid. In some embodiments, the peptide, antibody, lipid, carbohydrate, or polymer conjugated to the oligonucleotide confers targeting specificity of the oligonucleotide to specific types of cells (e.g., cancer cells, etc.). In some embodiments, the peptide, antibody, lipid, carbohydrate, or polymer conjugated to the oligonucleotide confers stability of the oligonucleotide in vitro, ex vivo, or in vivo. For example, the oligonucleotide can be conjugated with polyethylene glycol (PEG) or endosomolytic agent to decrease immunogenicity or degradation. In some embodiments, the peptide, antibody, lipid, carbohydrate, or polymer conjugated to the oligonucleotide to facilitate the oligonucleotide for entering cell. In some embodiments, the peptide, antibody, lipid, carbohydrate, or polymer conjugated to the oligonucleotide to facilitate and release to the oligonucleotide in the cell. In some embodiments, the peptide, antibody, lipid, carbohydrate, or polymer conjugated to the oligonucleotide comprises at least one targeting moiety for targeting the cell. Non-limiting examples of the targeting moiety comprises a signaling peptide, a chemokine, a chemokine receptor, an adhesion molecule, an antigen, or an antibody.

The linker for conjugating the oligonucleotide to the peptide, antibody, lipid, or polymer can be any linker that connects biomolecules. In some embodiments, a linker described herein is a cleavable linker or a non-cleavable linker. In some instances, the linker is a cleavable linker. In other instances, the linker is a non-cleavable linker. In some cases, the linker is a non-polymeric linker. A non-polymeric linker refers to a linker that does not contain a repeating unit of monomers generated by a polymerization process. In some embodiments, the linker comprises a peptide moiety. In some instances, the peptide moiety comprises at least 2, 3, 4, 5, or 6 more amino acid residues. In some embodiments, the linker comprises a benzoic acid group, or its derivatives thereof. In some embodiments, the linker can comprise nucleic acid linker such as DNA linker. In such case, the peptide, antibody, lipid, or polymer can be conjugated on one end of the nucleic acid linker or intercalated into the nucleic acid base pairing of the nucleic acid linker. In some embodiments, the linker can be a peptide linker. The peptide linker can be flexible (e.g., poly-glycine linker) or rigid (e.g., EAAAK repeat linker, SEQ ID NO: 196). In some embodiments, the peptide linker can be cleaved (e.g., a disulfide bond). In some embodiments, the linker comprises polymers such PEG, polylactic acid (PLA), or polyacrylic acid (PAA).

### Methods of Treatment

Disclosed herein, in some embodiments, are methods of modulating KRAS-mediated signaling pathway in a cancer cell by treating or contacting the cancer cell with a composition comprising antisense oligonucleotide, composition, or pharmaceutical composition described herein, thereby reducing expression of SOS1 or SOS2 protein or mRNA in the cancer cell. Also disclosed herein, in some embodiments, are methods of treating a subject in need thereof by administrating a therapeutic effective amount of the oligonucleotide, composition, or pharmaceutical composition described herein to the subject. In some embodiments, the method comprises administering at least one additional active ingredient as a combination therapy for treating the disease or condition. In some embodiments, the method treats the subject by modulating gene expression or signaling pathway expression in the subject. In some embodiments, the method comprises decreasing gene expression by contacting endogenous nucleic acid (e.g., endogenous mRNA) with the oligonucleotide described herein. In some embodiments, the method comprises decreasing SOS1, SOS2, or a combination of SOS1 and SOS2 in the subject or in the cancer cell by contacting mRNA of SOS1 or SOS2 with the oligonucleotide described herein, where the binding of the oligonucleotide to the mRNA recruits endogenous nuclease for degradation of the mRNA. In some embodiments, the method comprises decreasing expression of signaling pathway such as KRAS-mediated signaling pathway. In some embodiments, the method comprises decreasing expression of a gene in or the activity of KRAS-RAF-MEK-ERK signaling pathway.

In some embodiments, the disease or condition described herein is a genetic disorder. In some embodiments, the genetic disorder is Noonan syndrome. In some embodiments, the genetic disorder is hereditary gingival fibromatosis type 1.

In some embodiments, the oligonucleotide, composition, or pharmaceutical composition can be administered to the subject alone (e.g., standalone treatment). In some embodiments, the oligonucleotide, composition, or pharmaceutical composition is administered in combination with an additional agent (e.g., an active ingredient described herein). In some cases, the additional agent as used herein is administered alone. The oligonucleotide, composition, or pharmaceutical composition and the additional agent can be administered together or sequentially. Non-limiting examples of the additional agent comprise N-(2-(4-(4-bis(2-chloroethyl)aminophenyl) butyryl)aminoethyl)-5-(4-amidinophenyl)-2-furanecarboxamide hydrochloride; Allyl isothiocyanate; Benzyl isothiocyanate; Phenethyl isothiocyanate; Belinostat; Berberin; Casticin; Chrysin; Bufalin; Fisetin; Fucoidan; Galic acid; Gemcitabine; Guizhi Fuling Decoction; JOTO1007; Quercetin; Rasfonin; 2,3,7,8-tetrachlorodibenzodioxin; Triptolide; 4-Hydroxybutenolide; or a combination thereof. In some embodiments, the at least one additional agent or ingredient comprises an KRAS inhibitor. In some embodiments, the at least one additional agent or ingredient comprises RAF inhibitor. In some embodiments, the at least one additional agent or ingredient comprises a MEK inhibitor. In some embodiments, the at least one additional agent or ingredient comprises an ERK inhibitor. In some embodiments, the at least one additional agent or ingredient comprises a SH2 inhibitor. The combination therapies can be administered within the same day, or can be administered one or more days, weeks, months, or years apart.

In some embodiments, the oligonucleotide, composition, or pharmaceutical composition is a first-line treatment for the disease or condition. In some embodiments, the oligonucleotide, composition, or pharmaceutical composition is a second-line, third-line, or fourth-line treatment. In some embodiments, the oligonucleotide, composition, or pharmaceutical composition comprises at least one, two, three, four, five, six, seven, eight, nine, 10, 20, 30 or more oligonucleotide. In general, method disclosed herein comprises administering the oligonucleotide, composition, or pharmaceutical composition by oral administration. However, in some instances, method comprises administering the oligonucleotide, composition, or pharmaceutical composition by intraperitoneal injection. In some instances, the method comprises administering the pharmaceutical composition in the form of an anal suppository. In some instances, the method comprises administering the oligonucleotide, composition, or pharmaceutical composition by intravenous (“i.v.”) administration. It is conceivable that one can also administer the oligonucleotide, composition, or pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

Suitable dose and dosage administrated to a subject is determined by factors including, but no limited to, the particular the oligonucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some embodiments, the administration of the oligonucleotide, composition, or pharmaceutical composition described herein is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In some embodiments, the administration of the oligonucleotide, composition, or pharmaceutical composition described herein inhibits growth of the tumor by at least 10%, 15%, 20%, 30%, 40%, 50%, or more. In some embodiments, the administration of the oligonucleotide, composition, or pharmaceutical composition described herein at a dose that inhibits growth of the tumor by at least 10%, 15%, 20%, 30%, 40%, 50%, or more. In some embodiments, the administration of the oligonucleotide, composition, or pharmaceutical composition described herein at a schedule that inhibits growth of the tumor by at least 10%, 15%, 20%, 30%, 40%, 50%, or more. In some embodiments, the administration of the oligonucleotide, composition, or pharmaceutical composition described herein at a dose and a schedule that inhibits growth of the tumor by at least 10%, 15%, 20%, 30%, 40%, 50%, or more.

In some embodiments, the administration of the oligonucleotide, composition, or pharmaceutical composition described herein to the subject in a dose that is sufficient to inhibit growth of the tumor. In some embodiments, the administration of the oligonucleotide, composition, or pharmaceutical composition described herein to the subject in a schedule that is sufficient to inhibit growth of the tumor. In some embodiments, the administration of the oligonucleotide, composition, or pharmaceutical composition described herein to the subject in a dose and a schedule that are sufficient to inhibit growth of the tumor.

In certain embodiments, where the subject's condition does not improve, upon the doctor's discretion the administration of the pharmaceutical composition is administered chronically, that is, for an extended period of time, including throughout the duration of the subject's life in order to ameliorate or otherwise control or limit the symptoms of the subject's disease or condition. In certain embodiments wherein a subject's status does improve, the dose of the pharmaceutical composition being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of the pharmaceutical composition being administered can be temporarily reduced or temporarily suspended for a certain length of time (i.e., a “drug diversion”). In specific embodiments, the length of the pharmaceutical composition diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during the pharmaceutical composition diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

In some embodiments, the disease or condition described herein is a cancer. In some embodiments, the cancer is associated with SOS1. In some embodiments, the cancer is associated with SOS2. In some embodiments, the cancer is associated with KRAS. In some embodiments, the cancer is associated with an abnormality of KRAS-mediated signaling pathway. In some embodiments, the cancer is a lung cancer, a pancreatic cancer, or a colon cancer. Other non-limiting examples of the cancer can include Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sézary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

### Pharmaceutical Compositions

Described herein, in some embodiments, is a pharmaceutical composition comprising the oligonucleotide or the composition described herein. Pharmaceutical composition, as used herein, refers to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical compositions described herein comprise the oligonucleotide, the compositions, the cells contacted with the oligonucleotide or contacted with the composition comprising the oligonucleotide, or a combination thereof.

In some embodiments, the composition of the pharmaceutical composition described herein comprises at least one active ingredient. In some embodiments, the at least one active ingredient functions as a combination therapy with an oligonucleotide described herein. In some embodiments, the at least one active ingredient comprises a cancer therapeutic agent. In some embodiments, the at least one active ingredient comprises a modulator of the KRAS-mediated signaling pathway (e.g., a KRAS-RAF-MEK-ERK signaling pathway). In some embodiments, the at least one active ingredient comprises an inhibitor of the KRAS-mediated signaling pathway. In some embodiments, the at least one active ingredient comprises an inhibitor of SOS. In some embodiments, the at least one active ingredient comprises an inhibitor of SOS1. In some embodiments, the at least one active ingredient comprises an inhibitor of SOS2. In some embodiments, the at least one active ingredient comprises an inhibitor of SOS1 or SOS2. In some embodiments, the at least one active ingredient comprises an inhibitor of both SOS1 and SOS2. In some embodiments, the inhibitor of SOS comprises at least one oligonucleotide described herein. In some embodiments, the inhibitor of SOS does not comprise an oligonucleotide described herein.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, pharmaceutical compositions exist in unsolvated form or in solvated forms with pharmaceutically acceptable solvents such as water, ethanol, and the like. The solvated forms of the pharmaceutical compositions are also considered to be disclosed herein.

In some embodiments, a pharmaceutical composition exists as a tautomer. All tautomers are included within the scope of the agents presented herein. As such, it is to be understood that a pharmaceutical composition or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they can be regarded as different isomeric forms of the same compound.

In some embodiments, a pharmaceutical composition exists as an enantiomer, diastereomer, or other steroisomeric form. The agents disclosed herein include all enantiomeric, diastereomeric, and epimeric forms as well as mixtures thereof.

In some embodiments, pharmaceutical compositions described herein can be prepared as prodrugs. A “prodrug” refers to an agent that is converted into the parent drug in vivo. Prodrugs are often useful because, in some situations, they can be easier to administer than the parent drug. They may, for instance, be bioavailable by oral administration whereas the parent is not. The prodrug may also have improved solubility in pharmaceutical compositions over the parent drug. An example, without limitation, of a prodrug would be a pharmaceutical composition described herein, which is administered as an ester (the “prodrug”) to facilitate transmittal across a cell membrane where water solubility is detrimental to mobility but which then is metabolically hydrolyzed to the carboxylic acid, the active enzyme, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the pharmaceutical composition. In certain embodiments, a prodrug is enzymatically metabolized by one or more steps or processes to the biologically, pharmaceutically or therapeutically active form of the pharmaceutical composition.

Prodrug forms of the pharmaceutical compositions, wherein the prodrug is metabolized in vivo to produce an agent as set forth herein are included within the scope of the claims. Prodrug forms of the herein described pharmaceutical compositions, wherein the prodrug is metabolized in vivo to produce an agent as set forth herein are included within the scope of the claims. In some cases, some of the pharmaceutical compositions described herein can be a prodrug for another derivative or active compound. In some embodiments described herein, hydrazones are metabolized in vivo to produce a pharmaceutical composition.

### Kits

Described herein, in some embodiments, are kits for using the oligonucleotide, the compositions, or the pharmaceutical compositions described herein. In some embodiments, the kits disclosed herein may be used to treat a disease or condition in a subject. In some embodiments, the kit comprises an assemblage of materials or components apart from the oligonucleotide, the composition, or the pharmaceutical composition. In some embodiments, the kit comprises the components for assaying and selecting for suitable oligonucleotide for treating a disease or a condition. In some embodiments, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the oligonucleotide. In some embodiments, the kit comprises instructions thawing or otherwise restoring biological activity of the oligonucleotide, which may have been cryopreserved or lyophilized during storage or transportation. In some embodiments, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example the oligonucleotide, the composition, or the pharmaceutical composition may be in dissolved, dehydrated, or lyophilized form. The components are typically contained in suitable packaging material(s).

### Description of Embodiments

1. An antisense oligonucleotide that inhibits the expression of son of sevenless 1 (SOS1) or son of sevenless 2 (SOS2) mRNA, wherein the antisense oligonucleotide comprises 10 to 30 linked nucleotides and has a sequence that is complementary to SOS1 or SOS2 mRNA, and wherein the oligonucleotide has at least 8 contiguous nucleotides of any one of SEQ ID NOS: 1 to 189.

2. The antisense oligonucleotide of embodiment 1, wherein the oligonucleotide is at least 12 nucleotides in length.

3. The antisense oligonucleotide of embodiment 2, wherein the oligonucleotide is at least 14 nucleotides in length.

4. The antisense oligonucleotide of embodiment 2, wherein the oligonucleotide is from 10 to 24 nucleotides in length, or 10 to 16 nucleotides in length, or 12 to 16 nucleotides in length.

5. The antisense oligonucleotide of embodiment 4, wherein the oligonucleotide is 12, 13, 14, 15, or 16 nucleotides in length.

6. The antisense oligonucleotide of embodiment 5, wherein the oligonucleotide is 14 nucleotides in length.

7. The antisense oligonucleotide of any one of embodiments 1 to 6, wherein the oligonucleotide comprises at least 8, at least 10, or at least 12 contiguous nucleobases of any one of SEQ ID NOs: 1 to 189.

8. The antisense oligonucleotide of embodiment 7, wherein the oligonucleotide comprises or consists of a nucleobase sequence of any one of SEQ ID Nos: 1 to 189.

9. The antisense oligonucleotide of any one of embodiments 1 to 8, wherein the oligonucleotide has a nucleobase sequence and structure selected from Table 4.

10. The antisense oligonucleotide of embodiment 8 or 9, wherein the oligonucleotide has a nucleobase sequence selected from SEQ ID NOs: 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 and 187.

11. The antisense oligonucleotide of any one of embodiments 1 to 10, wherein the antisense oligonucleotide has a stretch of at least 6 DNA nucleotides sufficient to recruit RNaseH.

12. The antisense oligonucleotide of embodiment 11, wherein the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides.

13. The antisense oligonucleotide of embodiment 12, wherein the 5′ and 3′ segments are each 3 nucleotides in length, and the 5′ and 3′ segments flank an internal sequence of 8 DNA nucleotides.

14. The antisense oligonucleotide of embodiment 13, wherein one or more nucleotides of the 5′ segment and the 3′ segment comprise 2′-O substituents, optionally where all of the nucleotides of the 5′ segment and the 3′ segment comprise 2′-O substituents.

15. The antisense oligonucleotide of embodiment 14, wherein the 2′-O substituents are independently selected from 2′-O methyl, 2′-O ethyl, 2′-O methoxyethyl (MOE), and a bridged nucleotide having a 2′ to 4′ bridge.

16. The antisense oligonucleotide of embodiment 15, wherein the bridged nucleotide has a methylene bridge (LNA) or a constrained ethyl bridge (cEt).

17. The antisense oligonucleotide of any one of embodiments 1 to 16, wherein the antisense oligonucleotide has a modified backbone.

18. The antisense oligonucleotide of embodiment 17, wherein the antisense oligonucleotide comprises one or more phosphorothioate or phosphorodithioate nucleotides.

19. The antisense oligonucleotide of embodiment 18, wherein the oligonucleotide is fully phosphorothioate or phosphorodithioate linked.

20. The antisense oligonucleotide of embodiment 19, wherein the oligonucleotide is fully phosphorothioate linked.

21. The antisense oligonucleotide of any one of embodiments 1 to 20, wherein cytidine nucleobases in the antisense oligonucleotide are 5-methyl cytidine.

22. The antisense oligonucleotide of any one of embodiments 1 to 21, further comprising a cell penetrating moiety.

23. The antisense oligonucleotide of embodiment 22, wherein the cell penetrating moiety is conjugated directly or indirectly at the 3′ end of the oligonucleotide, optionally though a linker.

24. The antisense oligonucleotide of embodiment 22 or 23, wherein the moiety comprises a sterol conjugate or fatty acid conjugate, which is optionally cholesteryl, palmitoyl, or stearyl conjugate.

25. The antisense oligonucleotide of any one of embodiments 1 to 24, wherein the composition does not comprise any encapsulation or transfection reagent.

26. The antisense oligonucleotide of any one of embodiments 1 to 25, wherein the antisense oligonucleotide is encapsulated in a particle.

27. The antisense oligonucleotide of embodiment 26, wherein the particle is a liposome, polymeric nanoparticle, or lipid nanoparticle.

28. The antisense oligonucleotide of any one of embodiments 1 to 27, wherein the oligonucleotide is formulated for parenteral administration.

29. A pharmaceutical composition comprising an antisense oligonucleotide of any one of embodiments 1 to 28, and a pharmaceutically acceptable carrier or vehicle.

30. A method for treating a subject having a condition associated with aberrant expression or activity of SOS1 or SOS2, comprising administering an effective amount of the composition of any one of embodiments 1 to 28, or the composition of embodiment 29, to the subject.

31. The method of embodiment 30, wherein the subject has Noonan syndrome.

32. The method of embodiment 30, wherein the subject has hereditary gingival fibromatosis type 1.

33. The method of embodiment 30, wherein the subject has a malignancy associated with aberrant expression of SOS1 or SOS2.

34. The method of embodiment 33, wherein the malignancy associated with aberrant expression of SOS1 or SOS2 is non-metastatic.

35 The method of embodiment 33, wherein the malignancy associated with aberrant expression of SOS1 or SOS2 is metastatic.

36. The method of any one of embodiments 33 to 35, wherein the malignancy is a carcinoma.

37. The method of embodiment 36, wherein the malignancy is breast cancer, cervical cancer, pancreatic cancer, squamous cell carcinoma, head and neck cancer, thyroid cancer, gastric cancer, colon cancer, or liver cancer.

38. The method of embodiment 36 or 37, wherein the malignancy is cervical squamous cell carcinoma, endocervical adenocarcinoma, cholangiocarcinoma, esophageal carcinoma, colon adenocarcinoma, oral squamous cell carcinoma, head and neck squamous cell carcinoma, lung adenocarcinoma, lung squamous cell carcinoma, non-small cell lung cancer, liver hepatocellular carcinoma, lung squamous cell carcinoma, rectum adenocarcinoma, stomach adenocarcinoma, thyroid carcinoma, or pancreatic adenocarcinoma.

9. The method of any one of embodiments 30 to 38, wherein the composition is administered parenterally.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular.

Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Modulating Expressions of SOS

### Cell Culture Condition and In Vitro Transfection

ATCC cells were grown in RPMI-1640. The media were supplemented with 10% fetal bovine serum, 100 U/ml penicillin and 100 U/ml streptomycin (Pen-strep, or PS), and cell were incubated at 37° C. in a humidified incubator containing 5% CO₂. For antisense treatment, cells were plated at 70% confluency the previous day in 96-well plates. On the day of transfection, cells were washed once with OptiMEM medium and incubated in 80 μL OptiMEM. Transfection mixture was prepared in OptiMEM by mixing the antisense and the Lipofectamine RNAiMax transfection reagents at desired concentration, and 20 μL of the transfection mixture was added into each well, and incubated for 2 hours. At the end of the 2 hours, 10 μL of serum were added in to well, and made up the volume to 200 μL with respective culture medium for the cell line. Alternatively, media were replaced 2 hours post transfection or on the next day. The ASO treatment could also be done without transfection reagent. In this case, the ASOs were diluted with OptiMEM, and added into the cell culture in a volume less than 5% of the entire volume.

mRNA Knockdown Detection

Cells were harvested by lysis at 48 hours post transfection. The lysis and the follow up mRNA detection were conducted according to the Quantigen assay specified by the manufacturer (ThermoFisher).

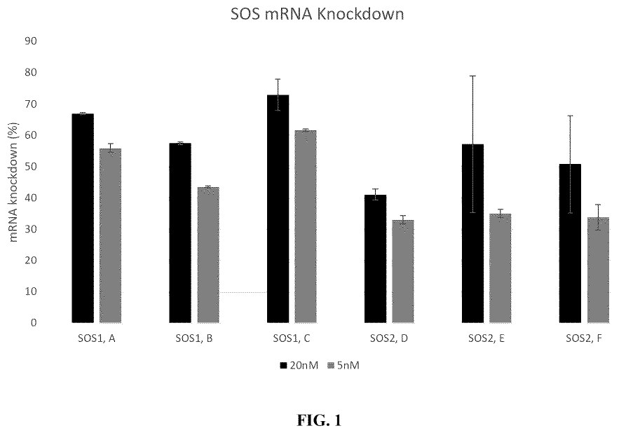

The ability of ASOs to knockdown desired mRNA was assessed as follows. NCI-H358 (ATCC) cells were plated in clear, flat bottom 96-well plate at 15,000 cells per well in RPMI1640 with 10% FBS for overnight. Cells were transfected with ASO at 5 nM and 20 nM complexed with RNAiMax according to manufacturer's instructions (Thermo Fisher). After 3 hours of transfection, transfection mixture was removed and replenished with RPMI1640+10% FBS and incubated for 48 hours. The mRNA quantitation was performed using QuantiGene from Thermo Fisher according to its instructions. The results for the initial study with initial test were in FIG. 1 and sequences of A-F were in Table 1. In FIG. 1, on the x-axis, SOS1 or SOS2 depicts the mRNA being measured, and A-F depicts the ASO used as shown in Table 1. In order to accommodate screening studies and normalize inter- and intra-experimental variation, SEQ ID NO: 1 was selected as the benchmark ASO in the screening process implemented in all plates and to normalize the mRNA knockdown.

TABLE 1
ASO nucleic acid sequence for normalizing the experimental variation

| ASO# | Nucleic acid sequence | SEQ ID NO: |
|---|---|---|
| A | GCTCGAATGATCGGAA | 194 |
| B | ATTCGACTCACCACAG | 195 |
| C | TCGCCTATTGACTGCA | 196 |
| D | TCGCTCCTCTACATCT | 197 |
| E | TTCCCATCCATCGATA | 198 |
| F | CCCATCAAATGCACCA | 199 |

For screening ASO in SOS2 knockdown function, the procedure is the same, except 10 nM ASOs were used in the transfection conditions and Cell line MIA PaCa-2 (ATCC)] was used (noted in Table 2). The mRNA knockdown data were recorded as both actual mRNA knockdown percentage and normalized with SEQ ID NO: 1 activity to facilitate comparison. The results are listed in Table 2.

TABLE 2
Normalized SOS2 mRNA knockdown

| | | MIA PaCa-2 | |
|---|---|---|---|
| Cell Line | | 10 nM | |
| ASO conc. | | SOS2 mRNA | mRNA Knockdown |
| SEQ ID NO | ASO# | knockdown (%) | Normalized to SEQ ID NO: 1 |
| 1 | 1086 | 31 | 100 |
| 2 | 386 | 23 | 75 |
| 3 | 394 | 26 | 83 |
| 4 | 562 | 8 | 27 |
| 5 | 573 | 40 | 128 |
| 6 | 578 | 28 | 89 |
| 7 | 581 | 35 | 113 |
| 8 | 583 | 19 | 60 |
| 9 | 657 | −4 | −13 |
| 10 | 660 | 37 | 120 |
| 11 | 662 | 29 | 94 |
| 12 | 776 | −31 | −98 |
| 13 | 840 | 17 | 56 |
| 14 | 882 | −21 | −68 |
| 15 | 905 | 38 | 122 |
| 16 | 938 | 47 | 151 |
| 17 | 952 | 27 | 86 |
| 18 | 957 | 32 | 104 |
| 19 | 960 | 35 | 114 |
| 20 | 1038 | 53 | 172 |
| 21 | 1043 | 30 | 96 |
| 22 | 1069 | 11 | 35 |
| 23 | 1174 | 38 | 122 |
| 24 | 1245 | 12 | 40 |
| 25 | 1247 | 9 | 28 |
| 26 | 1251 | 29 | 93 |
| 27 | 1307 | 57 | 184 |
| 28 | 1326 | −24 | −76 |
| 29 | 1345 | 8 | 24 |
| 30 | 1377 | −4 | −12 |
| 31 | 1407 | 36 | 117 |
| 32 | 1410 | −5 | −17 |
| 33 | 1452 | 21 | 68 |
| 34 | 1472 | 5 | 17 |
| 35 | 1512 | 6 | 19 |
| 36 | 1514 | 22 | 72 |
| 37 | 1573 | 6 | 20 |
| 38 | 1607 | −14 | −46 |
| 39 | 1636 | 23 | 73 |
| 40 | 1639 | 5 | 15 |
| 41 | 1643 | 33 | 106 |
| 42 | 1647 | 51 | 162 |
| 43 | 1654 | 43 | 138 |
| 44 | 1700 | 4 | 13 |
| 45 | 1732 | 20 | 66 |
| 46 | 1764 | −8 | −26 |
| 47 | 1804 | 47 | 150 |
| 48 | 1816 | 39 | 103 |
| 49 | 1820 | 48 | 127 |
| 50 | 1859 | 19 | 50 |
| 51 | 1933 | 43 | 113 |
| 52 | 1937 | −16 | −44 |
| 53 | 1985 | 25 | 67 |
| 54 | 2008 | −3 | −8 |
| 55 | 2011 | 1 | 4 |
| 56 | 2036 | 34 | 90 |
| 57 | 2054 | 25 | 65 |
| 58 | 2084 | 17 | 45 |
| 59 | 2118 | −1 | −3 |
| 60 | 2142 | 6 | 15 |
| 61 | 2172 | 34 | 90 |
| 62 | 2202 | −12 | −31 |
| 63 | 2227 | 34 | 90 |
| 64 | 2248 | 32 | 85 |
| 65 | 2297 | 33 | 87 |
| 66 | 2304 | 43 | 114 |
| 67 | 2306 | 18 | 47 |
| 68 | 2310 | 3 | 7 |
| 69 | 2340 | 19 | 50 |
| 70 | 2343 | 27 | 72 |
| 71 | 2445 | 29 | 76 |
| 72 | 2471 | 27 | 70 |
| 73 | 2478 | 40 | 106 |
| 74 | 2480 | 40 | 106 |
| 75 | 2483 | 25 | 65 |
| 76 | 2486 | 7 | 17 |
| 77 | 2489 | 20 | 53 |
| 78 | 2525 | 22 | 59 |
| 79 | 2533 | 22 | 60 |
| 80 | 2564 | 38 | 101 |
| 81 | 2616 | 29 | 76 |
| 82 | 2637 | 28 | 75 |
| 83 | 2641 | 39 | 103 |
| 84 | 2643 | 33 | 88 |
| 85 | 2681 | 42 | 113 |
| 86 | 2690 | 36 | 97 |
| 87 | 2757 | 37 | 97 |
| 88 | 2761 | 34 | 91 |
| 89 | 2763 | 25 | 68 |
| 90 | 2768 | 24 | 64 |
| 91 | 2825 | 22 | 58 |
| 92 | 2854 | 18 | 47 |
| 93 | 2886 | 29 | 78 |
| 94 | 2915 | 29 | 94 |
| 95 | 2921 | 34 | 108 |
| 96 | 3002 | 30 | 95 |
| 97 | 3031 | 26 | 84 |
| 98 | 3033 | 30 | 95 |
| 99 | 3102 | 0 | 0 |
| 100 | 3106 | 1 | 5 |
| 101 | 3114 | 33 | 104 |
| 102 | 3139 | 14 | 46 |
| 103 | 3222 | 4 | 14 |
| 104 | 3227 | −4 | −12 |
| 105 | 3232 | −2 | −6 |
| 106 | 3240 | 2 | 7 |
| 107 | 3263 | 23 | 74 |
| 108 | 3270 | 19 | 61 |
| 109 | 3335 | −25 | −79 |
| 110 | 3350 | 23 | 74 |
| 111 | 3354 | 28 | 89 |
| 112 | 3371 | 24 | 76 |
| 113 | 3406 | −13 | −42 |
| 114 | 3416 | 9 | 29 |
| 115 | 3418 | −12 | −38 |
| 116 | 3425 | 31 | 100 |
| 117 | 3452 | 23 | 73 |
| 118 | 3466 | 16 | 50 |
| 119 | 3492 | 31 | 98 |
| 120 | 3496 | 2 | 7 |
| 121 | 3498 | −2 | −7 |
| 122 | 3501 | 5 | 15 |
| 123 | 3519 | 14 | 43 |
| 124 | 3526 | 22 | 69 |
| 125 | 3588 | 25 | 79 |
| 126 | 3641 | 10 | 31 |
| 127 | 3711 | 1 | 3 |
| 128 | 3747 | −5 | −15 |
| 129 | 3812 | 21 | 67 |
| 130 | 3823 | 20 | 63 |
| 131 | 3893 | 20 | 62 |
| 132 | 3991 | 11 | 36 |
| 133 | 4046 | 6 | 20 |
| 134 | 4062 | 17 | 54 |
| 135 | 4097 | 27 | 87 |
| 136 | 4101 | 28 | 90 |
| 137 | 4103 | 16 | 50 |
| 138 | 4105 | 30 | 97 |
| 139 | 4121 | 37 | 119 |
| 140 | 4132 | 14 | 40 |
| 141 | 4142 | 28 | 78 |
| 142 | 4187 | 15 | 41 |
| 143 | 4192 | 31 | 87 |
| 144 | 4199 | 31 | 87 |
| 145 | 4214 | 21 | 59 |
| 146 | 4218 | 17 | 48 |
| 147 | 4222 | −10 | −27 |
| 148 | 4242 | 24 | 68 |
| 149 | 4253 | 1 | 2 |
| 150 | 4259 | 37 | 103 |
| 151 | 4265 | 5 | 14 |
| 152 | 4290 | 20 | 56 |
| 153 | 4318 | 20 | 55 |
| 154 | 4393 | 25 | 71 |
| 155 | 4418 | −6 | −17 |
| 156 | 4463 | 15 | 42 |
| 157 | 4523 | 37 | 102 |
| 158 | 4580 | 32 | 89 |
| 159 | 4584 | 25 | 71 |
| 160 | 4635 | 21 | 57 |
| 161 | 4710 | 31 | 86 |
| 162 | 4766 | 14 | 40 |
| 163 | 4768 | 3 | 10 |
| 164 | 4773 | 36 | 101 |
| 165 | 4913 | 18 | 50 |
| 166 | 4953 | 10 | 29 |
| 167 | 5078 | −2 | −5 |
| 168 | 5103 | 21 | 59 |
| 169 | 5111 | 28 | 78 |
| 170 | 5114 | 17 | 47 |
| 171 | 5118 | 25 | 70 |
| 172 | 5122 | 34 | 94 |
| 173 | 5133 | 16 | 44 |
| 174 | 5167 | 13 | 35 |
| 175 | 5172 | 20 | 55 |
| 176 | 5178 | 24 | 68 |
| 177 | 5187 | 21 | 59 |
| 178 | 5192 | 31 | 87 |
| 179 | 5208 | 35 | 96 |
| 180 | 5211 | 28 | 79 |
| 181 | 5214 | 43 | 119 |
| 182 | 5303 | 8 | 21 |
| 183 | 5416 | −21 | −57 |
| 184 | 5470 | 2 | 4 |
| 185 | 5481 | 1 | 2 |
| 186 | 1039 | 37 | 0 |
| 187 | 1039a | 30 | 96.8 |
| 188 | 1040 | 7 | 22.6 |
| 189 | 1040a | 0 | 0 |

### Cell Proliferation Assay

After the antisense treatment, cells were monitored at 3 or 5 days post transfection. Cell proliferation was monitored by CellTiter-Glo assay according to the protocol specified by the manufacturer (Promega).

### Inhibition of Cell Growth

The ability of ASOs to inhibit cell growth was assessed as follows. MIA PaCa-2 cells were plated in clear 384-well plates (S-Bio, #MS-9384UZ) at 800 cells per well in RPMI1640 with 10% FBS for overnight. Cells were treated with 2.5 μM ASO (noted in Table 3). After 5 days, cell proliferation was determined by measuring total ATP content using the Cell Titer Glo reagent (Promega, G7570) according to manufacturer's instructions. MIA PaCa-2 cell carries KRAS mutation. The results for growth inhibition are shown in Table 3.

In some cases, even if a SOS2 ASO may not exhibit substantial cell growth inhibition activity, it can still be combined with other SOS1/SOS2 ASOs to achieve dual SOS1 and SOS2 inhibition. For example, a SOS2 ASO may be combined with SEQ ID NO: 1 for dual SOS1/SOS2 inhibition.

TABLE 3
Cell growth inhibition

| Cell Line | | MIA PaCa-2 | |
|---|---|---|---|
| ASO conc. | | 2.5 uM | |
| SEQ | | 3D Growth | 3D Growth Inhibition (%) |
| ID NO | ASO# | Inhibition (%) | Normalized to SEQ ID NO: 1 |
| 1 | 1086 | 43 | 100 |
| 2 | 386 | −18 | −40 |
| 3 | 394 | −9 | −20 |
| 4 | 562 | −13 | −28 |
| 5 | 573 | −3 | −7 |
| 6 | 578 | −8 | −18 |
| 7 | 581 | 15 | 32 |
| 8 | 583 | −12 | −26 |
| 9 | 657 | −7 | −15 |
| 10 | 660 | 19 | 41 |
| 11 | 662 | −7 | −16 |
| 12 | 776 | −4 | −8 |
| 13 | 840 | 0 | 0 |
| 14 | 882 | −4 | −9 |
| 15 | 905 | −10 | −21 |
| 16 | 938 | 9 | 19 |
| 17 | 952 | −5 | −10 |
| 18 | 957 | 6 | 14 |
| 19 | 960 | −10 | −21 |
| 20 | 1038 | 50 | 109 |
| 21 | 1043 | −8 | −17 |
| 22 | 1069 | −2 | −4 |
| 23 | 1174 | 0 | 0 |
| 24 | 1245 | −5 | −11 |
| 25 | 1247 | −1 | −2 |
| 26 | 1251 | −1 | −3 |
| 27 | 1307 | −3 | −7 |
| 28 | 1326 | −5 | −11 |
| 29 | 1345 | 5 | 10 |
| 30 | 1377 | 1 | 3 |
| 31 | 1407 | 21 | 46 |
| 32 | 1410 | 0 | 0 |
| 33 | 1452 | −5 | −10 |
| 34 | 1472 | −1 | −3 |
| 35 | 1512 | −1 | −2 |
| 36 | 1514 | 1 | 3 |
| 37 | 1573 | −7 | −16 |
| 38 | 1607 | −6 | −13 |
| 39 | 1636 | −4 | −9 |
| 40 | 1639 | −3 | −7 |
| 41 | 1643 | −27 | −58 |
| 42 | 1647 | 27 | 58 |
| 43 | 1654 | −2 | −5 |
| 44 | 1700 | 4 | 9 |
| 45 | 1732 | 2 | 5 |
| 46 | 1764 | −8 | −17 |
| 47 | 1804 | −6 | −14 |
| 48 | 1816 | 0 | 0 |
| 49 | 1820 | −3 | −7 |
| 50 | 1859 | −14 | −31 |
| 51 | 1933 | −3 | −6 |
| 52 | 1937 | −3 | −6 |
| 53 | 1985 | −7 | −14 |
| 54 | 2008 | −20 | −44 |
| 55 | 2011 | −9 | −19 |
| 56 | 2036 | 39 | 85 |
| 57 | 2054 | −7 | −16 |
| 58 | 2084 | −6 | −13 |
| 59 | 2118 | −9 | −19 |
| 60 | 2142 | 7 | 14 |
| 61 | 2172 | 5 | 10 |
| 62 | 2202 | −4 | −9 |
| 63 | 2227 | 6 | 13 |
| 64 | 2248 | 0 | −1 |
| 65 | 2297 | 16 | 34 |
| 66 | 2304 | 13 | 28 |
| 67 | 2306 | 8 | 17 |
| 68 | 2310 | 4 | 8 |
| 69 | 2340 | 2 | 4 |
| 70 | 2343 | 8 | 18 |
| 71 | 2445 | −6 | −12 |
| 72 | 2471 | 14 | 31 |
| 73 | 2478 | −16 | −36 |
| 74 | 2480 | −12 | −26 |
| 75 | 2483 | −8 | −18 |
| 76 | 2486 | −9 | −20 |
| 77 | 2489 | −11 | −23 |
| 78 | 2525 | 7 | 15 |
| 79 | 2533 | 0 | 0 |
| 80 | 2564 | −1 | −3 |
| 81 | 2616 | −29 | −62 |
| 82 | 2637 | 1 | 2 |
| 83 | 2641 | 2 | 5 |
| 84 | 2643 | −2 | −5 |
| 85 | 2681 | 6 | 13 |
| 86 | 2690 | −15 | −32 |
| 87 | 2757 | −3 | −6 |
| 88 | 2761 | 0 | −1 |
| 89 | 2763 | 27 | 58 |
| 90 | 2768 | 6 | 14 |
| 91 | 2825 | 2 | 4 |
| 92 | 2854 | −4 | −9 |
| 93 | 2886 | 2 | 4 |
| 94 | 2915 | 19 | 47 |
| 95 | 2921 | 17 | 42 |
| 96 | 3002 | 10 | 25 |
| 97 | 3031 | 11 | 28 |
| 98 | 3033 | 8 | 21 |
| 99 | 3102 | 45 | 112 |
| 100 | 3106 | 1 | 1 |
| 101 | 3114 | 37 | 93 |
| 102 | 3139 | 34 | 84 |
| 103 | 3222 | 20 | 51 |
| 104 | 3227 | 13 | 32 |
| 105 | 3232 | 7 | 17 |
| 106 | 3240 | 2 | 6 |
| 107 | 3263 | 35 | 86 |
| 108 | 3270 | 3 | 7 |
| 109 | 3335 | −1 | −3 |
| 110 | 3350 | 26 | 65 |
| 111 | 3354 | 3 | 8 |
| 112 | 3371 | 15 | 37 |
| 113 | 3406 | 5 | 13 |
| 114 | 3416 | 11 | 27 |
| 115 | 3418 | 3 | 8 |
| 116 | 3425 | −4 | 19 |
| 117 | 3452 | 15 | 45 |
| 118 | 3466 | 7 | 48 |
| 119 | 3492 | 9 | 53 |
| 120 | 3496 | 4 | 16 |
| 121 | 3498 | 9 | 45 |
| 122 | 3501 | 22 | 76 |
| 123 | 3519 | 10 | 45 |
| 124 | 3526 | −9 | −24 |
| 125 | 3588 | −5 | −12 |
| 126 | 3641 | 15 | 38 |
| 127 | 3711 | 14 | 35 |
| 128 | 3747 | 12 | 29 |
| 129 | 3812 | 14 | 36 |
| 130 | 3823 | 5 | 14 |
| 131 | 3893 | 7 | 17 |
| 132 | 3991 | 7 | 17 |
| 133 | 4046 | 13 | 32 |
| 134 | 4062 | 14 | 36 |
| 135 | 4097 | 16 | 39 |
| 136 | 4101 | 12 | 31 |
| 137 | 4103 | 15 | 36 |
| 138 | 4105 | 16 | 40 |
| 139 | 4121 | 21 | 53 |
| 140 | 4132 | 15 | 37 |
| 141 | 4142 | 15 | 38 |
| 142 | 4187 | 29 | 71 |
| 143 | 4192 | 12 | 31 |
| 144 | 4199 | 7 | 16 |
| 145 | 4214 | 16 | 40 |
| 146 | 4218 | 6 | 16 |
| 147 | 4222 | 6 | 15 |
| 148 | 4242 | −1 | −3 |
| 149 | 4253 | 6 | 15 |
| 150 | 4259 | 2 | 4 |
| 151 | 4265 | 3 | 8 |
| 152 | 4290 | 9 | 23 |
| 153 | 4318 | 5 | 12 |
| 154 | 4393 | 6 | 14 |
| 155 | 4418 | 4 | 9 |
| 156 | 4463 | 4 | 9 |
| 157 | 4523 | 15 | 38 |
| 158 | 4580 | 43 | 108 |
| 159 | 4584 | 4 | 10 |
| 160 | 4635 | 7 | 16 |
| 161 | 4710 | 18 | 46 |
| 162 | 4766 | 14 | 34 |
| 163 | 4768 | 22 | 55 |
| 164 | 4773 | 8 | 19 |
| 165 | 4913 | −24 | −59 |
| 166 | 4953 | −1 | −2 |
| 167 | 5078 | −2 | −6 |
| 168 | 5103 | −2 | −5 |
| 169 | 5111 | −3 | −8 |
| 170 | 5114 | 10 | 25 |
| 171 | 5118 | 20 | 51 |
| 172 | 5122 | 7 | 17 |
| 173 | 5133 | −12 | −29 |
| 174 | 5167 | −6 | −14 |
| 175 | 5172 | 5 | 12 |
| 176 | 5178 | 19 | 48 |
| 177 | 5187 | −5 | −14 |
| 178 | 5192 | 6 | 15 |
| 179 | 5208 | 7 | 17 |
| 180 | 5211 | 10 | 25 |
| 181 | 5214 | 0 | 1 |
| 182 | 5303 | 3 | 8 |
| 183 | 5416 | 7 | 17 |
| 184 | 5470 | 3 | 7 |
| 185 | 5481 | 10 | 25 |
| 186 | 1039 | 0 | 0 |
| 187 | 1039a | 24 | 55.8 |
| 188 | 1040 | 0 | 0 |
| 189 | 1040a | 3 | 7.0 |

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

TABLE 4
Exemplary Antisense Oligonucleotides

| Oligo | | Sequence Identifier for |
|---|---|---|
| No. | Antisense Oligonucleotide | Nucleobase Sequence |
| 1086 | +A*+T*+T*T*C*A*T*A*A*T*G*+C*+C*+G | SEQ ID NO: 1 |
| 386 | +T*+G*+C*+A*C*T*T*G*T*T*C*+C*+T*+G | SEQ ID NO: 2 |
| 394 | +G*+A*+G*T*G*G*G*A*T*G*C*+A*+C*+T | SEQ ID NO: 3 |
| 562 | +T*+A*+G*C*A*G*A*T*T*G*T*+G*+C*+A | SEQ ID NO: 4 |
| 573 | +T*+C*+G*T*T*T*T*T*C*T*A*+T*+A*+G | SEQ ID NO: 5 |
| 578 | +C*+G*+T*T*T*T*C*G*T*T*T*+T*+T*+C | SEQ ID NO: 6 |
| 581 | +C*+T*+T*C*G*T*T*T*T*C*G*+T*+T*+T | SEQ ID NO: 7 |
| 583 | +T*+T*+C*T*T*C*G*T*T*T*T*+C*+G*+T | SEQ ID NO: 8 |
| 657 | +A*+T*+G*G*T*A*G*T*C*C*A*+C*+T*+T | SEQ ID NO: 9 |
| 660 | +T*+A*+C*A*T*G*G*T*A*G*T*+C*+C*+A | SEQ ID NO: 10 |
| 662 | +G*+A*+T*A*C*A*T*G*G*T*A*+G*+T*+C | SEQ ID NO: 11 |
| 776 | +G*+A*+C*A*C*T*T*T*A*A*T*+G*+T*+C | SEQ ID NO: 12 |
| 840 | +G*+A*+G*A*G*A*A*A*C*C*A*+A*+A*+C | SEQ ID NO: 13 |
| 882 | +A*+T*+A*G*T*A*G*T*T*T*A*+A*+T*+T | SEQ ID NO: 14 |
| 905 | +G*+C*+G*A*T*T*T*C*A*G*T*+T*+C*+T | SEQ ID NO: 15 |
| 938 | +A*+T*+A*T*T*T*A*A*T*T*C*+C*+C*+G | SEQ ID NO: 16 |
| 952 | +A*+C*+A*C*T*T*T*T*A*T*G*+A*+T*+C | SEQ ID NO: 17 |
| 957 | +T*+C*+G*A*A*A*C*A*C*T*T*+T*+T*+A | SEQ ID NO: 18 |
| 960 | +T*+T*+C*T*C*G*A*A*A*C*A*+C*+T*+T | SEQ ID NO: 19 |
| 1038 | +T*+T*+C*A*T*G*T*A*T*A*T*+C*+T*+G | SEQ ID NO: 20 |
| 1043 | +G*+T*+C*A*A*T*T*C*A*T*G*+T*+A*+T | SEQ ID NO: 21 |
| 1069 | +C*+T*+T*C*A*A*T*C*A*A*A*+C*+C*+T | SEQ ID NO: 22 |
| 1174 | +G*+T*+G*A*T*A*A*T*G*T*T*+T*+C*+A | SEQ ID NO: 23 |
| 1245 | +G*+T*+G*T*A*G*A*G*C*A*A*+C*+T*+G | SEQ ID NO: 24 |
| 1247 | +A*+A*+G*T*G*T*A*G*A*G*C*+A*+A*+C | SEQ ID NO: 25 |
| 1251 | +C*+T*+G*A*A*A*G*T*G*T*A*+G*+A*+G | SEQ ID NO: 26 |
| 1307 | +A*+G*+C*A*T*A*A*G*A*C*G*+T*+G*+G | SEQ ID NO: 27 |
| 1326 | +A*+C*+A*G*T*G*A*T*A*C*A*+C*+T*+G | SEQ ID NO: 28 |
| 1345 | +G*+T*+A*A*C*T*C*A*A*A*G*+T*+A*+G | SEQ ID NO: 29 |
| 1377 | +T*+T*+G*T*T*C*T*T*C*A*C*+T*+A*+C | SEQ ID NO: 30 |
| 1407 | +A*+A*+T*A*G*C*T*T*G*G*T*+T*+C*+A | SEQ ID NO: 31 |
| 1410 | +A*+G*+T*A*A*T*A*G*C*T*T*+G*+G*+T | SEQ ID NO: 32 |
| 1452 | +C*+T*+T*G*T*A*A*A*T*T*C*+G*+G*+T | SEQ ID NO: 33 |
| 1472 | +C*+G*+A*C*G*T*C*T*A*G*G*+T*+G*+A | SEQ ID NO: 34 |
| 1512 | +T*+A*+A*T*T*G*G*T*G*A*C*+T*+A*+T | SEQ ID NO: 35 |
| 1514 | +C*+T*+T*A*A*T*T*G*G*T*G*+A*+C*+T | SEQ ID NO: 36 |
| 1573 | +C*+C*+C*A*T*C*C*A*T*C*G*+A*+T*+A | SEQ ID NO: 37 |
| 1607 | +A*+A*+T*T*C*A*T*T*A*C*A*+A*+C*+A | SEQ ID NO: 38 |
| 1636 | +C*+G*+A*T*T*C*T*T*G*T*C*+A*+A*+T | SEQ ID NO: 39 |
| 1639 | +C*+A*+C*C*G*A*T*T*C*T*T*+G*+T*+C | SEQ ID NO: 40 |
| 1643 | +T*+T*+G*G*C*A*C*C*G*A*T*+T*+C*+T | SEQ ID NO: 41 |
| 1647 | +A*+T*+G*T*T*T*G*G*C*A*C*+C*+G*+A | SEQ ID NO: 42 |
| 1654 | +G*+C*+C*G*T*T*C*A*T*G*T*+T*+T*+G | SEQ ID NO: 43 |
| 1700 | +T*+G*+A*T*T*A*G*G*T*T*T*+A*+C*+A | SEQ ID NO: 44 |
| 1732 | +C*+A*+C*T*A*C*T*G*T*A*A*+C*+C*+T | SEQ ID NO: 45 |
| 1764 | +C*+A*+T*G*A*C*A*A*A*T*T*+T*+T*+T | SEQ ID NO: 46 |
| 1804 | +G*+C*+T*C*A*C*A*A*G*T*A*+T*+C*+T | SEQ ID NO: 47 |
| 1816 | +A*+T*+G*C*A*T*G*C*T*T*G*+T*+G*+C | SEQ ID NO: 48 |
| 1820 | +T*+C*+A*A*A*T*G*C*A*T*G*+C*+T*+T | SEQ ID NO: 49 |
| 1859 | +G*+C*+A*G*C*A*A*A*T*A*T*+T*+A*+T | SEQ ID NO: 50 |
| 1933 | +T*+T*+C*G*A*T*C*T*A*G*A*+G*+T*+A | SEQ ID NO: 51 |
| 1937 | +A*+A*+C*A*T*T*C*G*A*T*C*+T*+A*+G | SEQ ID NO: 52 |
| 1985 | +G*+G*+T*A*A*T*C*T*C*A*G*+T*+G*+G | SEQ ID NO: 53 |
| 2008 | +C*+A*+A*A*A*C*G*A*T*A*T*+A*+C*+T | SEQ ID NO: 54 |
| 2011 | +C*+T*+A*C*A*A*A*A*C*G*A*+T*+A*+T | SEQ ID NO: 55 |
| 2036 | +A*+C*+A*A*T*G*T*T*T*T*C*+C*+T*+C | SEQ ID NO: 56 |
| 2054 | +T*+G*+C*A*A*G*T*T*G*T*+C*+T*+T*C | SEQ ID NO: 57 |
| 2084 | +C*+C*+T*T*T*A*A*T*A*A*T*+G*+G*+G | SEQ ID NO: 58 |
| 2118 | +T*+G*+T*T*A*A*C*C*T*T*T*+C*+A*+A | SEQ ID NO: 59 |
| 2142 | +A*+T*+T*G*G*G*A*T*C*T*G*+C*+A*+T | SEQ ID NO: 60 |
| 2172 | +A*+C*+G*A*T*A*T*G*T*G*G*+T*+A*+A | SEQ ID NO: 61 |
| 2202 | +G*+C*+T*C*A*G*C*A*A*T*T*+C*+C*+T | SEQ ID NO: 62 |
| 2227 | +G*+A*+A*T*T*T*C*A*A*A*C*+C*+G*+T | SEQ ID NO: 63 |
| 2248 | +C*+G*+T*C*A*G*T*A*G*G*T*+T*+C*+T | SEQ ID NO: 64 |
| 2297 | +A*+G*+G*T*C*T*G*C*A*C*T*+G*+A*+T | SEQ ID NO: 65 |
| 2304 | +T*+C*+T*T*T*T*A*A*G*G*T*+C*+T*+G | SEQ ID NO: 66 |
| 2306 | +A*+A*+T*C*T*T*T*T*A*A*G*+G*+T*+C | SEQ ID NO: 67 |
| 2310 | +G*+C*+G*A*A*A*T*C*T*T*T*+T*+A*+A | SEQ ID NO: 68 |
| 2340 | +C*+C*+T*A*A*G*T*T*G*T*A*+C*+T*+G | SEQ ID NO: 69 |
| 2343 | +G*+A*+T*C*C*T*A*A*G*T*T*+G*+T*+A | SEQ ID NO: 70 |
| 2445 | +C*+C*+C*T*C*T*T*A*C*A*C*+T*+T*+G | SEQ ID NO: 71 |
| 2471 | +G*+A*+C*T*C*T*A*C*C*C*A*+T*+T*+T | SEQ ID NO: 72 |
| 2478 | +A*+G*+C*A*A*T*T*G*A*C*T*+C*+T*+A | SEQ ID NO: 73 |
| 2480 | +T*+T*+A*G*C*A*A*T*T*G*A*+C*+T*+C | SEQ ID NO: 74 |
| 2483 | +A*+T*+C*T*T*A*G*C*A*A*T*+T*+G*+A | SEQ ID NO: 75 |
| 2486 | +A*+T*+G*A*T*C*T*T*A*G*C*+A*+A*+T | SEQ ID NO: 76 |
| 2489 | +C*+T*+G*A*T*G*A*T*C*T*T*+A*+G*+C | SEQ ID NO: 77 |
| 2525 | +T*+G*+G*C*T*T*A*C*T*C*C*+G*+T*+T | SEQ ID NO: 78 |
| 2533 | +T*+A*+A*T*A*T*T*A*T*G*G*+C*+T*+T | SEQ ID NO: 79 |
| 2564 | +C*+A*+T*T*C*A*A*T*T*G*G*+T*+G*+G | SEQ ID NO: 80 |
| 2616 | +A*+T*+G*A*A*G*T*G*T*C*A*+T*+G*+A | SEQ ID NO: 81 |
| 2637 | +C*+T*+G*A*C*G*T*G*C*A*A*+T*+T*+T | SEQ ID NO: 82 |
| 2641 | +T*+C*+A*G*C*T*G*A*C*G*T*+G*+C*+A | SEQ ID NO: 83 |
| 2643 | +T*+G*+T*C*A*G*C*T*G*A*C*+G*+T*+G | SEQ ID NO: 84 |
| 2681 | +G*+A*+C*G*G*T*T*G*A*A*C*+T*+T*+T | SEQ ID NO: 85 |
| 2690 | +A*+C*+A*A*G*T*T*C*A*G*A*+C*+G*+G | SEQ ID NO: 86 |
| 2757 | +A*+T*+G*G*C*G*A*A*T*C*A*+T*+T*+T | SEQ ID NO: 87 |
| 2761 | +T*+G*+G*T*A*T*G*G*C*G*A*+A*+T*+C | SEQ ID NO: 88 |
| 2763 | +T*+G*+T*G*G*T*A*T*G*G*C*+G*+A*+A | SEQ ID NO: 89 |
| 2768 | +A*+G*+A*T*T*T*G*T*G*G*T*+A*+T*+G | SEQ ID NO: 90 |
| 2825 | +G*+C*+C*A*C*C*C*G*T*T*C*+T*+T*+C | SEQ ID NO: 91 |
| 2854 | +G*+C*+A*G*A*A*T*T*T*C*T*+A*+T*+A | SEQ ID NO: 92 |
| 2886 | +G*+C*+C*A*T*T*G*A*A*A*T*+T*+A*+T | SEQ ID NO: 93 |
| 2915 | +G*+A*+A*T*T*T*A*C*T*G*C*+A*+C*+T | SEQ ID NO: 94 |
| 2921 | +G*+A*+C*A*C*T*G*A*A*T*T*+T*+A*+C | SEQ ID NO: 95 |
| 3002 | +T*+G*+A*C*T*T*A*A*T*T*C*+C*+A*+C | SEQ ID NO: 96 |
| 3031 | +G*+T*+T*T*T*A*C*T*A*G*G*+T*+A*+T | SEQ ID NO: 97 |
| 3033 | +A*+A*+G*T*T*T*T*A*C*T*A*+G*+G*+T | SEQ ID NO: 98 |
| 3102 | +T*+T*+C*T*T*C*G*G*T*C*T*+T*+C*+A | SEQ ID NO: 99 |
| 3106 | +T*+C*+C*C*T*T*C*T*T*C*G*+G*+T*+C | SEQ ID NO: 100 |
| 3114 | +A*+T*+C*A*T*T*A*T*T*C*C*+C*+T*+T | SEQ ID NO: 101 |
| 3139 | +A*+A*+T*C*T*T*T*C*C*C*T*+T*+T*+C | SEQ ID NO: 102 |
| 3222 | +C*+C*+G*T*A*A*A*C*A*G*T*+A*+A*+G | SEQ ID NO: 103 |
| 3227 | +T*+C*+T*A*T*C*C*G*T*A*A*+A*+C*+A | SEQ ID NO: 104 |
| 3232 | +C*+T*+G*G*T*T*C*T*A*T*C*+C*+G*+T | SEQ ID NO: 105 |
| 3240 | +C*+C*+T*C*A*T*A*T*C*T*G*+G*+T*+T | SEQ ID NO: 106 |
| 3263 | +G*+G*+G*T*T*A*A*G*G*T*T*+T*+T*+C | SEQ ID NO: 107 |
| 3270 | +T*+C*+C*C*A*T*G*G*G*G*T*+T*+A*+A | SEQ ID NO: 108 |
| 3335 | +T*+T*+T*C*G*A*G*G*T*T*C*+A*+A*+T | SEQ ID NO: 109 |
| 3350 | +G*+G*+T*G*G*C*T*G*T*T*T*+G*+C*+A | SEQ ID NO: 110 |
| 3354 | +T*+C*+G*A*G*G*T*G*G*C*T*+G*+T*+T | SEQ ID NO: 111 |
| 3371 | +G*+T*+T*G*A*T*T*T*C*C*T*+A*+G*+G | SEQ ID NO: 112 |
| 3406 | +T*+G*+T*T*A*G*G*C*C*T*T*+A*+T*+T | SEQ ID NO: 113 |
| 3416 | +T*+G*+T*C*G*G*C*C*T*G*T*+G*+T*+T | SEQ ID NO: 114 |
| 3418 | +C*+A*+T*G*T*C*G*G*C*C*T*+G*+T*+G | SEQ ID NO: 115 |
| 3425 | +G*+T*+A*G*A*G*C*C*A*T*G*+T*+C*+G | SEQ ID NO: 116 |
| 3452 | +G*+T*+T*G*G*G*T*G*A*C*C*+T*+C*+G | SEQ ID NO: 117 |
| 3466 | +C*+T*+C*T*T*T*C*T*A*A*T*+G*+G*+T | SEQ ID NO: 118 |
| 3492 | +C*+C*+G*A*C*T*A*A*A*G*C*+T*+T*+A | SEQ ID NO: 119 |
| 3496 | +C*+A*+A*T*C*C*G*A*C*T*A*+A*+A*+G | SEQ ID NO: 120 |
| 3498 | +A*+G*+C*A*A*T*C*C*G*A*C*+T*+A*+A | SEQ ID NO: 121 |
| 3501 | +T*+T*+C*A*G*C*A*A*T*C*C*+G*+A*+C | SEQ ID NO: 122 |
| 3519 | +T*+G*+T*T*G*A*T*T*C*C*A*+G*+C*+T | SEQ ID NO: 123 |
| 3526 | +C*+T*+G*A*C*A*C*T*G*T*T*+G*+A*+T | SEQ ID NO: 124 |
| 3588 | +T*+A*+C*A*C*T*A*A*G*G*T*+C*+T*+G | SEQ ID NO: 125 |
| 3641 | +G*+G*+A*G*C*A*A*A*G*A*T*+G*+C*+T | SEQ ID NO: 126 |
| 3711 | +G*+G*+G*C*T*C*T*T*C*A*C*+T*+T*+A | SEQ ID NO: 127 |
| 3747 | +A*+A*+A*C*T*T*T*T*T*T*C*+G*+A*+G | SEQ ID NO: 128 |
| 3812 | +G*+G*+T*G*G*A*A*T*A*G*C*+A*+G*+G | SEQ ID NO: 129 |
| 3823 | +G*+A*+G*G*C*T*G*T*C*T*C*+G*+G*+T | SEQ ID NO: 130 |
| 3893 | +G*+G*+T*G*G*A*C*T*A*T*G*+C*+A*+G | SEQ ID NO: 131 |
| 3991 | +G*+T*+G*G*C*T*G*A*A*G*A*+T*+T*+A | SEQ ID NO: 132 |
| 4046 | +C*+A*+C*G*T*A*C*T*A*A*T*+G*+T*+C | SEQ ID NO: 133 |
| 4062 | +G*+C*+T*T*G*G*C*G*A*A*T*+T*+T*+G | SEQ ID NO: 134 |
| 4097 | +C*+G*+C*G*G*T*A*C*C*C*T*+T*+G*+G | SEQ ID NO: 135 |
| 4101 | +T*+C*+G*A*C*G*C*G*G*T*A*+C*+C*+C | SEQ ID NO: 136 |
| 4103 | +C*+A*+T*C*G*A*C*G*C*G*G*+T*+A*+C | SEQ ID NO: 137 |
| 4105 | +A*+G*+C*A*T*C*G*A*C*G*C*+G*+G*+T | SEQ ID NO: 138 |
| 4121 | +C*+T*+A*G*A*A*C*T*G*A*G*+C*+A*+C | SEQ ID NO: 139 |
| 4132 | +G*+A*+T*T*A*T*T*C*T*G*A*+C*+T*+A | SEQ ID NO: 140 |
| 4142 | +G*+G*+A*T*G*A*G*C*A*A*G*+A*+T*+T | SEQ ID NO: 141 |
| 4187 | +A*+G*+A*T*G*A*G*G*G*C*T*+T*+G*+A | SEQ ID NO: 142 |
| 4192 | +T*+T*+G*G*C*A*G*A*T*G*A*+G*+G*+G | SEQ ID NO: 143 |
| 4199 | +G*+G*+C*A*G*T*T*T*T*G*G*+C*+A*+G | SEQ ID NO: 144 |
| 4214 | +T*+T*+G*T*A*A*G*T*C*T*T*+T*+G*+G | SEQ ID NO: 145 |
| 4218 | +C*+C*+G*T*T*T*G*T*A*A*G*+T*+C*+T | SEQ ID NO: 146 |
| 4222 | +G*+C*+T*C*C*C*G*T*T*T*G*+T*+A*+A | SEQ ID NO: 147 |
| 4242 | +G*+T*+A*C*A*A*T*G*G*G*G*+G*+G*+T | SEQ ID NO: 148 |
| 4253 | +A*+A*+A*G*G*C*A*G*T*C*T*+G*+T*+A | SEQ ID NO: 149 |
| 4259 | +T*+C*+T*A*G*C*A*A*A*G*G*+C*+A*+G | SEQ ID NO: 150 |
| 4265 | +G*+C*+A*T*T*T*T*C*T*A*G*+C*+A*+A | SEQ ID NO: 151 |
| 4290 | +T*+G*+G*C*T*A*A*G*G*T*C*+A*+T*+T | SEQ ID NO: 152 |
| 4318 | +A*+T*+A*C*C*A*T*T*C*C*A*+G*+T*+G | SEQ ID NO: 153 |
| 4393 | +T*+G*+T*A*A*G*A*G*C*A*T*+T*+A*+T | SEQ ID NO: 154 |
| 4418 | +A*+A*+G*C*T*T*A*T*T*T*G*+A*+T*+C | SEQ ID NO: 155 |
| 4463 | +G*+A*+T*A*A*T*G*G*T*G*A*+A*+G*+G | SEQ ID NO: 156 |
| 4523 | +A*+T*+A*C*T*G*G*C*C*T*T*+T*+T*+G | SEQ ID NO: 157 |
| 4580 | +A*+T*+C*A*T*G*T*C*A*C*T*+T*+T*+A | SEQ ID NO: 158 |
| 4584 | +T*+G*+C*A*A*T*C*A*T*G*T*+C*+A*+C | SEQ ID NO: 159 |
| 4635 | +C*+A*+T*C*T*A*T*T*A*C*T*+C*+A*+A | SEQ ID NO: 160 |
| 4710 | +T*+G*+G*T*C*A*A*A*G*A*G*+G*+T*+T | SEQ ID NO: 161 |
| 4766 | +G*+T*+T*G*G*T*G*T*T*A*T*+T*+C*+A | SEQ ID NO: 162 |
| 4768 | +C*+A*+G*T*T*G*G*T*G*T*T*+A*+T*+T | SEQ ID NO: 163 |
| 4773 | +A*+A*+G*A*A*C*A*G*T*T*G*+G*+T*+G | SEQ ID NO: 164 |
| 4913 | +A*+T*+C*A*T*T*G*G*G*T*T*+A*+T*+G | SEQ ID NO: 165 |
| 4953 | +A*+A*+A*G*G*C*A*C*A*T*C*+T*+G*+A | SEQ ID NO: 166 |
| 5078 | +A*+T*+A*T*A*G*G*T*T*A*T*+T*+A*+G | SEQ ID NO: 167 |
| 5103 | +T*+A*+T*A*G*G*C*C*T*C*A*+A*+G*+T | SEQ ID NO: 168 |
| 5111 | +G*+A*+T*T*G*T*A*T*T*A*T*+A*+G*+G | SEQ ID NO: 169 |
| 5114 | +G*+C*+A*G*A*T*T*G*T*A*T*+T*+A*+T | SEQ ID NO: 170 |
| 5118 | +A*+G*+T*A*G*C*A*G*A*T*T*+G*+T*+A | SEQ ID NO: 171 |
| 5122 | +G*+T*+A*A*A*G*T*A*G*C*A*+G*+A*+T | SEQ ID NO: 172 |
| 5133 | +T*+T*+T*A*T*G*A*A*T*T*A*+G*+T*+A | SEQ ID NO: 173 |
| 5167 | +C*+A*+G*T*T*G*G*T*T*C*C*+T*+A*+T | SEQ ID NO: 174 |
| 5172 | +C*+A*+A*G*G*C*A*G*T*T*G*+G*+T*+T | SEQ ID NO: 175 |
| 5178 | +T*+G*+A*A*G*G*C*A*A*G*G*+C*+A*+G | SEQ ID NO: 176 |
| 5187 | +A*+C*+T*A*G*G*T*C*T*T*G*+A*+A*+G | SEQ ID NO: 177 |
| 5192 | +A*+A*+G*T*T*A*C*T*A*G*G*+T*+C*+T | SEQ ID NO: 178 |
| 5208 | +A*+C*+G*A*G*A*T*T*T*A*T*+A*+G*+A | SEQ ID NO: 179 |
| 5211 | +A*+A*+C*A*C*G*A*G*A*T*T*+T*+A*+T | SEQ ID NO: 180 |
| 5214 | +G*+T*+T*A*A*C*A*C*G*A*G*+A*+T*+T | SEQ ID NO: 181 |
| 5303 | +A*+C*+T*T*A*T*C*T*G*A*T*+T*+A*+A | SEQ ID NO: 182 |
| 5416 | +G*+T*+A*A*A*T*A*A*A*C*A*+A*+C*+G | SEQ ID NO: 183 |
| 5470 | +T*+T*+A*T*T*A*A*T*C*T*T*+G*+T*+T | SEQ ID NO: 184 |
| 5481 | +A*+C*+T*G*G*G*G*T*G*A*T*+T*+T*+A | SEQ ID NO: 185 |
| 1039 | +A*+T*+T*C*A*T*G*T*A*T*A*+T*+C*+T | SEQ ID NO: 186 |
| 1039a | +G*+T*+T*C*A*T*G*T*A*T*A*+T*+C*+T | SEQ ID NO: 187 |
| 1040 | +A*+A*+T*T*C*A*T*G*T*A*T*+A*+T*+C | SEQ ID NO: 188 |
| 1040a | +A*+G*+T*T*C*A*T*G*T*A*T*+A*+T*+C | SEQ ID NO: 189 |

“+” indicates LNA, and may be optionally replaced with 2′-MOE or 2′-OMe, or other bridged nucleotide such as cEt;
“*” indicates a phosphorothioate internucleotide linkage;
C nucleotides may be optionally 5-methyl C

TABLE 5
Exemplary mRNA sequence of SOS1 or SOS2

| | SOS1/ | |
|---|---|---|
| SEQ ID | SOS2 | |
| NO | mRNA | Nucleic acid sequence |
| 190 | SOS1/ | attagagaaaaaaggcgacaggcattctgaatagcaggagacagagacagaagtgatgtgtttgtag |
| | SOS2 | gaggcaagagcagttcattttgagtgagagatacagctagaaagataagtagcggtccgattgtgca |
| | mRNA | aggccatggataccaggttaaggactttggaatttatcctgggacgccagtgtgagttcttgaacag |
| | #1 | aaaagtccaggggcaagttcatcctactctcgagtctaatgatgatgctcttcagtatgttgaagaa |
| | | ttaattttgcaattattaaatatgctatgccaagctcagccccgaagtgcttcagatgtagaggaac |
| | | gtgttcaaaaaagtttccctcatccaattgataaatgggcaatagctgatgcccaatcagctattga |
| | | aaagaggaagcgaagaaaccctttatctctcccagtagaaaaaattcatcctttattaaaggaggtc |
| | | ctaggttataaaattgaccaccaggtttctgtttacatagtagcagtcttagaatacatttctgcag |
| | | acattttaaagctggttgggaattatgtaagaaatatacggcattatgaaattacaaaacaagatat |
| | | taaagtggcaatgtgtgctgacaaggtattgatggatatgtttcatcaagatgtagaagatattaat |
| | | atattatctttaactgacgaagagccttccacctcaggagaacaaacttactatgatttggtaaaag |
| | | catttatggcagaaattcgacaatatataagggaactaaatctaattataaaagtttttagagagcc |
| | | ctttgtctccaattcaaaattgttttcagctaatgatgtagaaaatatatttagtcgcatagtagat |
| | | atacatgaacttagtgtaaagttactgggccatatagaagatacagtagaaatgacagatgaaggca |
| | | gtccccatccactagtaggaagctgctttgaagacttagcagaggaactggcatttgatccatatga |
| | | atcgtatgctcgagatattttgcgacctggttttcatgatcgtttccttagtcagttatcaaagcct |
| | | ggggcagcactttatttgcagtcaataggcgaaggtttcaaagaagctgttcaatatgttttaccca |
| | | ggctgcttctggcccctgtttaccactgtctccattactttgaacttttgaagcagttagaagaaaa |
| | | aagtgaagatcaagaagacaaggaatgtttaaaacaagcaataacagctttgcttaatgttcagagt |
| | | ggtatggaaaaaatatgttctaaaagtcttgcaaaacgaagactgagtgaatctgcatgtcggtttt |
| | | atagtcagcaaatgaaggggaaacaactagcaatcaagaagatgaacgagattcagaagaatattga |
| | | tggttgggagggaaaagacattggacagtgttgtaatgaatttataatggaaggaactcttacacgt |
| | | gtaggagccaaacatgagagacacatatttctctttgatggcttaatgatttgctgtaaatcaaatc |
| | | atgggcagccaagacttcctggtgctagcaatgcagaatatcgtcttaaagaaaagttttttatgcg |
| | | aaaggtacaaattaatgataaagatgacaccaatgaatacaagcatgcttttgaaataattttaaaa |
| | | gatgaaaatagtgttatattttctgccaagtcagctgaagagaaaaacaattggatggcagcattga |
| | | tatctttacagtaccggagtacactggaaaggatgcttgatgtaacaatgctacaggaagagaaaga |
| | | ggagcagatgaggctgcctagtgctgatgtttatagatttgcagagcctgactctgaagagaatatt |
| | | atatttgaagagaacatgcagcccaaggctggaattccaattatcaaagcaggaactgttattaaac |
| | | ttatagagaggcttacgtaccatatgtacgcagatcccaattttgttcggacatttcttacaacata |
| | | cagatccttttgcaaacctcaagaactactgagtcttataatagaaaggtttgaaattccagagcct |
| | | gagccaacagaagctgatcgcatagctatagagaatggagatcaacccttgagtgcagaactgaaaa |
| | | gatttagaaaagaatatatacagcctgtgcaactgcgagtattaaatgtatgtcggcactgggtaga |
| | | gcaccacttctatgattttgaaagagatgcatatcttttgcaacgaatggaagaatttattggaaca |
| | | gtaagaggtaaagcaatgaaaaaatgggttgaatccatcactaaaataatccaaaggaaaaaaattg |
| | | caagagacaatggaccaggtcataatattacatttcagagttcacctcccacagttgagtggcatat |
| | | aagcagacctgggcacatagagacttttgacctgctcaccttacacccaatagaaattgctcgacaa |
| | | ctcactttacttgaatcagatctataccgagctgtacagccatcagaattagttggaagtgtgtgga |
| | | caaaagaagacaaagaaattaactctcctaatcttctgaaaatgattcgacataccaccaacctcac |
| | | tctgtggtttgagaaatgtattgtagaaactgaaaatttagaagaaagagtagctgtggtgagtcga |
| | | attattgagattctacaagtctttcaagagttgaacaactttaatggtgtccttgaggttgtcagtg |
| | | ctatgaattcatcacctgtttacagactagaccacacatttgagcaaataccaagtcgccagaagaa |
| | | aattttagaagaagctcatgaattgagtgaagatcactataagaaatatttggcaaaactcaggtct |
| | | attaatccaccatgtgtgcctttctttggaatttatctcactaatatcttgaaaacagaagaaggca |
| | | accctgaggtcctaaaaagacatggaaaagagcttataaactttagcaaaaggaggaaagtagcaga |
| | | aataacaggagagatccagcagtaccaaaatcagccttactgtttacgagtagaatcagatatcaaa |
| | | aggttctttgaaaacttgaatccgatgggaaatagcatggagaaggaatttacagattatcttttca |
| | | acaaatccctagaaatagaaccacgaaaccctaagcctctcccaagatttccaaaaaaatatagcta |
| | | tcccctaaaatctcctggtgttcgtccatcaaacccaagaccaggtaccatgaggcatcccacacct |
| | | ctgcagcaggagccaaggaaaattagttatagtaggatccctgaaagtgaaacagaaagtacagcat |
| | | ctgcaccaaattctccaagaacaccgttaacacctccgcctgcttctggtgcttccagtaccacaga |
| | | tgtttgcagtgtatttgattccgatcattcgagcccttttcactcaagcaatgataccgtctttatc |
| | | caagttactctgccccatggcccaagatctgcttctgtatcatctataagtttaaccaaaggcactg |
| | | atgaagtgcctgtccctcctcctgttcctccacgaagacgaccagaatctgccccagcagaatcttc |
| | | accatctaagattatgtctaagcatttggacagtcccccagccattcctcctaggcaacccacatca |
| | | aaagcctattcaccacgatattcaatatcagaccggacctctatctcagaccctcctgaaagccctc |
| | | ccttattaccaccacgagaacctgtgaggacacctgatgttttctcaagctcaccactacatctcca |
| | | acctccccctttgggcaaaaaaagtgaccatggcaatgccttcttcccaaacagcccttcccccttt |
| | | acaccacctcctcctcaaacaccttctcctcacggcacaagaaggcatctgccatcaccaccattga |
| | | cacaagaagtggaccttcattccattgctgggccgcctgttcctccacgacaaagcacttctcaaca |
| | | tatccctaaactccctccaaaaacttacaaaagggagcacacacacccatccatgcacagagatgga |
| | | ccaccactgttggagaatgcccattcttcctgagttcctctgtactgggatgtatattttcctagcc |
| | | ccaaatccattgctggcaatggatgcactgaatgtgccagcactgaggagttaaaatgagaactcca |
| | | aacactaacgactcttcttcaagatgcagtataagacaatgaattttaacctagatgtaattataca |
| | | atggaaatggtattccagtttagaatatggaaagaccgacctagaggaattggacaactgattgcac |
| | | ctgaaaatcataaagggactttttctggccaataggcaggagtcctctttttgtgaagtgatctttt |
| | | gtgaagtgatcattaaagggatggaaaacacagtctagtgtccagcaggcccacatgacagtttttg |
| | | taattcaaattatgcacttttaaaaaaaaaaacttaaacagggatcttaatatcttcctttgttttc |
| | | ctttgctttactcttctactttagaatattttcttaaaaatcactcaaaggactgtgaggaaaggct |
| | | gtggtacctgaccttgttgaaatcaaggcccggcactgtactacaggcctgtttacagattattacg |
| | | gtgaactgaatgggtaccgaggcttcaccaaagaggtacttttttgttgttgttgttgttttaggaa |
| | | taattgtaccaattttaagagcattccccccacctgtccccacacacccaaacaaaatgtggtggtg |
| | | ttgccttcaaaaaagagaagttttgtgtcattaacatgacagaagaactttttaaaaaaaaataact |
| | | gtcaactattctatttgcatttaggagactgttcatctatgctagattgtcattttccctccttctc |
| | | ccacagaagtttactggtagtccatgtcatggctcgtagctatccctctaaccataccatggaaatg |
| | | caggcacccaatgtgaaaaggagcacttgctgggcatcactgacaccgctcatgttttacacatagt |
| | | tgagtaatcagcatatctagaattatcttgcattgcctaaatcatatgtatatagtgaatgttatat |
| | | aatatacctggcaggtctgttttaatttaattgaataaagatacaaatactttgtttggctggcata |
| | | tattaaattattatatgaagaaaatggttgattcttatttcttttagttgaaaaacacacaaaaaca |
| | | ctaaacataaaagcacgttttgtggtacgccttggtttctagttctggatatatgtattaatcttaa |
| | | agaatgatctaaagtcacagcttggcatataggaagatacttggttagaataggatgggtatctttt |
| | | aagcagaactttgtaagtaagttttaagttcctaagcaacaagtatacagtttgttaaggtttatat |
| | | tgaacattctaccttgcttgaggttttctgctttatcaaaactattcataatagaacatctacctgc |
| | | atcattactatcataatatggattattcataaagcgtatgtactgttcttctgagtctaacaaatac |
| | | tgtgactgaaatgcatgcaccttgttgaagagaaaacattttattaatctccactataatgtctgtg |
| | | aaatgtgcagtattccatccttagagagtttatctggttcatcagcatctgcatgtatcctctgaat |
| | | aatgaagcaacatttcagcaattctgttaaccacaccattagagacatttttagaagtgctgcaaca |
| | | tggtgggaacctagtactttaataatttcctatgatatttattagagcataaaatgtacatcacgat |
| | | agtacagtatgtagtcctttaagaccagaaggtatgttaactgcaccatcattaagcagtctatcct |
| | | attttcttatttatttgttagaattgaatcctttctcaatatctgtaccattcctatgatggtagcc |
| | | attcttctctttataaaatatttcctctttgaaaagcgtcctaaaacattaatttcaaatcccctct |
| | | tggatgcaaagtgtccctaagaccatccattcaacagtatttatgtgagccaacttactagagccgc |
| | | tacaactaatcatgatagacagtccagaatctccctggactttatactgggccagcctcattgtccc |
| | | taagatttctctttttatgtctcatggattacttctctttttaattgccccaggcacattccaaatg |
| | | agaacctggtaaaagtcacattggacccccttcagtatgtcttcggctaatgtgttagccttcaata |
| | | aggtgataaccccttttcaacctatctagtctataaagaggaaaaattcagatttggagttctaatc |
| | | ctatttctcccaaagggaactattcttctaccccatggttctaggagaccacttcactgtgcaacag |
| | | ttttacatgcattgtggatggaggatatttttattctgaactgttacatttaggtaaaaatattttc |
| | | tgcagtggtaactgattagaaaaatgccaattggatgcccttaggtggaggtgagaaaatggcatcc |
| | | ttgccttcttctcaatatgaaacattaactagttgacaaatttatccttgtagtaatgaaaatctat |
| | | ttaatcagggaccagaaatggctgaggagataaatgcatcattacaaaattctgcttttgaatcctg |
| | | gacattacaagggggtaaatgcagcatgactttttgttaaccacattccaaaatgtggaacatttct |
| | | tttagaaatgaaaatatttcaaggctgatgtattttaagactacacattatcaggaaacatacattg |
| | | agagttcgcttaattaaaggttgttgggcatcaaattatgtttagtaggttactattctctaacaac |
| | | tcaaggatgctttaatggatctgaatttgacaaagagcatgccacactaatactacagtcaacaaca |
| | | gcccagagaacaattactatgtcagctggaggctatattatgattctaaattcttaaaggttttttt |
| | | ccctccataaatcaaaaattaccttatgtaaaccaaaaattagttggtatttatggtcatgatctta |
| | | attctcaagtttagcttaatcttgtatttcattgtttgtcttctaatatgacagcttaaattcagat |
| | | ttttaagtgactcagcaaaataggaggagtgtcccaatttattagtgttgtacatattgaagaaaac |
| | | ctttttgttccttcagatttagaaagaaacagtttaaccatttatttcttggtattctgctgctgtg |
| | | aataggtttacttattcttttaacatatattgtgtgactacgcaagtataggtcctgttgtattgca |
| | | ttaatctttaccagtaactaaacatcacaaggttaatattggtttggctgaaagaattatgcagtaa |
| | | agttatttataagggaacatgatgactttattcaatatttttcttctttgaaacatctcattactaa |
| | | cttttaagattatttcataatcccttatacatgagccaatgaaatattttgagctctacttaagaag |
| | | catgaagtctatattataaatctaaacaacaaaagcacttgtaacttgtttagtaaattccatgcct |
| | | tattttccatttttgacaccgtaaagtgcattttctgtcgactgtgagcaaatttcccttttgcctt |
| | | taatgaaaatagtgcattaagtagccaattgctgcagaattataaagcaaattagctaacagtgggt |
| | | cacattggactaccactcaaagaaacaagatgcataacagatgatggaaggtacatgtggtgtggct |
| | | tagggagagcgcacctgtgccagatgcatgccactttcagtagctgccaaatgtgcctttaattaag |
| | | aacatctctaatttttttcttcagatcaagtcagcatttggggtcgggatttgggcagggaactttg |
| | | gggtgcaaatatctggtgaaaccaccagagaatttgtttgtcaaagtgacatgtataattttagttg |
| | | agacgtgtttgtgtatatatgtgtatttgcctttgtttagtagcacggttatttgctaaaataattg |
| | | tcaaaaaattgctctgtcggtcggtctttgatgtaagaacaaagtgtggtctatacatctaatatgg |
| | | cctttgtacctaaccatgttcataggtaatctttgtactctgtgtgcagcagtatttggtttgcatt |
| | | taaagtgaaaataacggctgttatttttctggcattactaagataaactgaaaaataataaagaaaa |
| | | atgccttacatagcccacaa |
| 191 | SOS1/ | agtagcgagcaggttcagttcgctagtaggaagctccagcgctacaccggcggcggcggtggcggcg |
| | SOS2 | gcggccgcggcggcacctcagggcagccagattccccaacaacgacctccggtccttccccttcccc |
| | mRNA | gcgagacctccgccccagctccgccccgcccggggctcttgcgtttcggagtcccaactacgcggag |
| | #2 | cgggcgccgcggccggagcgtccctccacacgtcctcccccagccgccagctccgccgcggggccag |
| | | accgggcccgggagtgcagccccggcgggcgggcggcggccgcggcagagggcagcccggggcggcg |
| | | ccggcgggccggggaggggcggggggcctggccccggggcggtggtgcgggatgccgccgcctccgg |
| | | gccgcgtccgttctccacggctggtacctgtgtcgggtgggtggccaggcgcgggcctcgcccccca |
| | | gccccctcgccagggctagcccggctgcgcggcgcccggagggggccgggccgtccggtggggccgc |
| | | ggccctgttccgcgctgcgagctcgccctctcgcggctccctggcccggccgccgccgcccctctcc |
| | | ccgcccagaggcgccccgggggcaccatgcaggcgcagcagctgccctacgagtttttcagcgaaga |
| | | gaacgcgcccaagtggcggggactactggtgcctgcgctgaaaaaggtccaggggcaagttcatcct |
| | | actctcgagtctaatgatgatgctcttcagtatgttgaagaattaattttgcaattattaaatatgc |
| | | tatgccaagctcagccccgaagtgcttcagatgtagaggaacgtgttcaaaaaagtttccctcatcc |
| | | aattgataaatgggcaatagctgatgcccaatcagctattgaaaagaggaagcgaagaaacccttta |
| | | tctctcccagtagaaaaaattcatcctttattaaaggaggtcctaggttataaaattgaccaccagg |
| | | tttctgtttacatagtagcagtcttagaatacatttctgcagacattttaaagctggttgggaatta |
| | | tgtaagaaatatacggcattatgaaattacaaaacaagatattaaagtggcaatgtgtgctgacaag |
| | | gtattgatggatatgtttcatcaagatgtagaagatattaatatattatctttaactgacgaagagc |
| | | cttccacctcaggagaacaaacttactatgatttggtaaaagcatttatggcagaaattcgacaata |
| | | tataagggaactaaatctaattataaaagtttttagagagccctttgtctccaattcaaaattgttt |
| | | tcagctaatgatgtagaaaatatatttagtcgcatagtagatatacatgaacttagtgtaaagttac |
| | | tgggccatatagaagatacagtagaaatgacagatgaaggcagtccccatccactagtaggaagctg |
| | | ctttgaagacttagcagaggaactggcatttgatccatatgaatcgtatgctcgagatattttgcga |
| | | cctggttttcatgatcgtttccttagtcagttatcaaagcctggggcagcactttatttgcagtcaa |
| | | taggcgaaggtttcaaagaagctgttcaatatgttttacccaggctgcttctggcccctgtttacca |
| | | ctgtctccattactttgaacttttgaagcagttagaagaaaaaagtgaagatcaagaagacaaggaa |
| | | tgtttaaaacaagcaataacagctttgcttaatgttcagagtggtatggaaaaaatatgttctaaaa |
| | | gtcttgcaaaacgaagactgagtgaatctgcatgtcggttttatagtcagcaaatgaaggggaaaca |
| | | actagcaatcaagaagatgaacgagattcagaagaatattgatggttgggagggaaaagacattgga |
| | | cagtgttgtaatgaatttataatggaaggaactcttacacgtgtaggagccaaacatgagagacaca |
| | | tatttctctttgatggcttaatgatttgctgtaaatcaaatcatgggcagccaagacttcctggtgc |
| | | tagcaatgcagaatatcgtcttaaagaaaagttttttatgcgaaaggtacaaattaatgataaagat |
| | | gacaccaatgaatacaagcatgcttttgaaataattttaaaagatgaaaatagtgttatattttctg |
| | | ccaagtcagctgaagagaaaaacaattggatggcagcattgatatctttacagtaccggagtacact |
| | | ggaaaggatgcttgatgtaacaatgctacaggaagagaaagaggagcagatgaggctgcctagtgct |
| | | gatgtttatagatttgcagagcctgactctgaagagaatattatatttgaagagaacatgcagccca |
| | | aggctggaattccaattatcaaagcaggaactgttattaaacttatagagaggcttacgtaccatat |
| | | gtacgcagatcccaattttgttcggacatttcttacaacatacagatccttttgcaaacctcaagaa |
| | | ctactgagtcttataatagaaaggtttgaaattccagagcctgagccaacagaagctgatcgcatag |
| | | ctatagagaatggagatcaacccttgagtgcagaactgaaaagatttagaaaagaatatatacagcc |
| | | tgtgcaactgcgagtattaaatgtatgtcggcactgggtagagcaccacttctatgattttgaaaga |
| | | gatgcatatcttttgcaacgaatggaagaatttattggaacagtaagaggtaaagcaatgaaaaaat |
| | | gggttgaatccatcactaaaataatccaaaggaaaaaaattgcaagagacaatggaccaggtcataa |
| | | tattacatttcagagttcacctcccacagttgagtggcatataagcagacctgggcacatagagact |
| | | tttgacctgctcaccttacacccaatagaaattgctcgacaactcactttacttgaatcagatctat |
| | | accgagctgtacagccatcagaattagttggaagtgtgtggacaaaagaagacaaagaaattaactc |
| | | tcctaatcttctgaaaatgattcgacataccaccaacctcactctgtggtttgagaaatgtattgta |
| | | gaaactgaaaatttagaagaaagagtagctgtggtgagtcgaattattgagattctacaagtctttc |
| | | aagagttgaacaactttaatggtgtccttgaggttgtcagtgctatgaattcatcacctgtttacag |
| | | actagaccacacatttgagcaaataccaagtcgccagaagaaaattttagaagaagctcatgaattg |
| | | agtgaagatcactataagaaatatttggcaaaactcaggtctattaatccaccatgtgtgcctttct |
| | | ttggaatttatctcactaatatcttgaaaacagaagaaggcaaccctgaggtcctaaaaagacatgg |
| | | aaaagagcttataaactttagcaaaaggaggaaagtagcagaaataacaggagagatccagcagtac |
| | | caaaatcagccttactgtttacgagtagaatcagatatcaaaaggttctttgaaaacttgaatccga |
| | | tgggaaatagcatggagaaggaatttacagattatcttttcaacaaatccctagaaatagaaccacg |
| | | aaaccctaagcctctcccaagatttccaaaaaaatatagctatcccctaaaatctcctggtgttcgt |
| | | ccatcaaacccaagaccaggtaccatgaggcatcccacacctctgcagcaggagccaaggaaaatta |
| | | gttatagtaggatccctgaaagtgaaacagaaagtacagcatctgcaccaaattctccaagaacacc |
| | | gttaacacctccgcctgcttctggtgcttccagtaccacagatgtttgcagtgtatttgattccgat |
| | | cattcgagcccttttcactcaagatctgcttctgtatcatctataagtttaaccaaaggcactgatg |
| | | aagtgcctgtccctcctcctgttcctccacgaagacgaccagaatctgccccagcagaatcttcacc |
| | | atctaagattatgtctaagcatttggacagtcccccagccattcctcctaggcaacccacatcaaaa |
| | | gcctattcaccacgatattcaatatcagaccggacctctatctcagaccctcctgaaagccctccct |
| | | tattaccaccacgagaacctgtgaggacacctgatgttttctcaagctcaccactacatctccaacc |
| | | tccccctttgggcaaaaaaagtgaccatggcaatgccttcttcccaaacagcccttccccctttaca |
| | | ccacctcctcctcaaacaccttctcctcacggcacaagaaggcatctgccatcaccaccattgacac |
| | | aagaagtggaccttcattccattgctgggccgcctgttcctccacgacaaagcacttctcaacatat |
| | | ccctaaactccctccaaaaacttacaaaagggagcacacacacccatccatgcacagagatggacca |
| | | ccactgttggagaatgcccattcttcctgagttcctctgtactgggatgtatattttcctagcccca |
| | | aatccattgctggcaatggatgcactgaatgtgccagcactgaggagttaaaatgagaactccaaac |
| | | actaacgactcttcttcaagatgcagtataagacaatgaattttaacctagatgtaattatacaatg |
| | | gaaatggtattccagtttagaatatggaaagaccgacctagaggaattggacaactgattgcacctg |
| | | aaaatcataaagggactttttctggccaataggcaggagtcctctttttgtgaagtgatcttttgtg |
| | | aagtgatcattaaagggatggaaaacacagtctagtgtccagcaggcccacatgacagtttttgtaa |
| | | ttcaaattatgcacttttaaaaaaaaaaacttaaacagggatcttaatatcttcctttgttttcctt |
| | | tgctttactcttctactttagaatattttcttaaaaatcactcaaaggactgtgaggaaaggctgtg |
| | | gtacctgaccttgttgaaatcaaggcccggcactgtactacaggcctgtttacagattattacggtg |
| | | aactgaatgggtaccgaggcttcaccaaagaggtacttttttgttgttgttgttgttttaggaataa |
| | | ttgtaccaattttaagagcattccccccacctgtccccacacacccaaacaaaatgtggtggtgttg |
| | | ccttcaaaaaagagaagttttgtgtcattaacatgacagaagaactttttaaaaaaaaataactgtc |
| | | aactattctatttgcatttaggagactgttcatctatgctagattgtcattttccctccttctccca |
| | | cagaagtttactggtagtccatgtcatggctcgtagctatccctctaaccataccatggaaatgcag |
| | | gcacccaatgtgaaaaggagcacttgctgggcatcactgacaccgctcatgttttacacatagttga |
| | | gtaatcagcatatctagaattatcttgcattgcctaaatcatatgtatatagtgaatgttatataat |
| | | atacctggcaggtctgttttaatttaattgaataaagatacaaatactttgtttggctggcatatat |
| | | taaattattatatgaagaaaatggttgattcttatttcttttagttgaaaaacacacaaaaacacta |
| | | aacataaaagcacgttttgtggtacgccttggtttctagttctggatatatgtattaatcttaaaga |
| | | atgatctaaagtcacagcttggcatataggaagatacttggttagaataggatgggtatcttttaag |
| | | cagaactttgtaagtaagttttaagttcctaagcaacaagtatacagtttgttaaggtttatattga |
| | | acattctaccttgcttgaggttttctgctttatcaaaactattcataatagaacatctacctgcatc |
| | | attactatcataatatggattattcataaagcgtatgtactgttcttctgagtctaacaaatactgt |
| | | gactgaaatgcatgcaccttgttgaagagaaaacattttattaatctccactataatgtctgtgaaa |
| | | tgtgcagtattccatccttagagagtttatctggttcatcagcatctgcatgtatcctctgaataat |
| | | gaagcaacatttcagcaattctgttaaccacaccattagagacatttttagaagtgctgcaacatgg |
| | | tgggaacctagtactttaataatttcctatgatatttattagagcataaaatgtacatcacgatagt |
| | | acagtatgtagtcctttaagaccagaaggtatgttaactgcaccatcattaagcagtctatcctatt |
| | | ttcttatttatttgttagaattgaatcctttctcaatatctgtaccattcctatgatggtagccatt |
| | | cttctctttataaaatatttcctctttgaaaagcgtcctaaaacattaatttcaaatcccctcttgg |
| | | atgcaaagtgtccctaagaccatccattcaacagtatttatgtgagccaacttactagagccgctac |
| | | aactaatcatgatagacagtccagaatctccctggactttatactgggccagcctcattgtccctaa |
| | | gatttctctttttatgtctcatggattacttctctttttaattgccccaggcacattccaaatgaga |
| | | acctggtaaaagtcacattggacccccttcagtatgtcttcggctaatgtgttagccttcaataagg |
| | | tgataaccccttttcaacctatctagtctataaagaggaaaaattcagatttggagttctaatccta |
| | | tttctcccaaagggaactattcttctaccccatggttctaggagaccacttcactgtgcaacagttt |
| | | tacatgcattgtggatggaggatatttttattctgaactgttacatttaggtaaaaatattttctgc |
| | | agtggtaactgattagaaaaatgccaattggatgcccttaggtggaggtgagaaaatggcatccttg |
| | | ccttcttctcaatatgaaacattaactagttgacaaatttatccttgtagtaatgaaaatctattta |
| | | atcagggaccagaaatggctgaggagataaatgcatcattacaaaattctgcttttgaatcctggac |
| | | attacaagggggtaaatgcagcatgactttttgttaaccacattccaaaatgtggaacatttctttt |
| | | agaaatgaaaatatttcaaggctgatgtattttaagactacacattatcaggaaacatacattgaga |
| | | gttcgcttaattaaaggttgttgggcatcaaattatgtttagtaggttactattctctaacaactca |
| | | aggatgctttaatggatctgaatttgacaaagagcatgccacactaatactacagtcaacaacagcc |
| | | cagagaacaattactatgtcagctggaggctatattatgattctaaattcttaaaggtttttttccc |
| | | tccataaatcaaaaattaccttatgtaaaccaaaaattagttggtatttatggtcatgatcttaatt |
| | | ctcaagtttagcttaatcttgtatttcattgtttgtcttctaatatgacagcttaaattcagatttt |
| | | taagtgactcagcaaaataggaggagtgtcccaatttattagtgttgtacatattgaagaaaacctt |
| | | tttgttccttcagatttagaaagaaacagtttaaccatttatttcttggtattctgctgctgtgaat |
| | | aggtttacttattcttttaacatatattgtgtgactacgcaagtataggtcctgttgtattgcatta |
| | | atctttaccagtaactaaacatcacaaggttaatattggtttggctgaaagaattatgcagtaaagt |
| | | tatttataagggaacatgatgactttattcaatatttttcttctttgaaacatctcattactaactt |
| | | ttaagattatttcataatcccttatacatgagccaatgaaatattttgagctctacttaagaagcat |
| | | gaagtctatattataaatctaaacaacaaaagcacttgtaacttgtttagtaaattccatgccttat |
| | | tttccatttttgacaccgtaaagtgcattttctgtcgactgtgagcaaatttcccttttgcctttaa |
| | | tgaaaatagtgcattaagtagccaattgctgcagaattataaagcaaattagctaacagtgggtcac |
| | | attggactaccactcaaagaaacaagatgcataacagatgatggaaggtacatgtggtgtggcttag |
| | | ggagagcgcacctgtgccagatgcatgccactttcagtagctgccaaatgtgcctttaattaagaac |
| | | atctctaatttttttcttcagatcaagtcagcatttggggtcgggatttgggcagggaactttgggg |
| | | tgcaaatatctggtgaaaccaccagagaatttgtttgtcaaagtgacatgtataattttagttgaga |
| | | cgtgtttgtgtatatatgtgtatttgcctttgtttagtagcacggttatttgctaaaataattgtca |
| | | aaaaattgctctgtcggtcggtctttgatgtaagaacaaagtgtggtctatacatctaatatggcct |
| | | ttgtacctaaccatgttcataggtaatctttgtactctgtgtgcagcagtatttggtttgcatttaa |
| | | agtgaaaataacggctgttatttttctggcattactaagataaactgaaaaataataaagaaaaatg |
| | | ccttacatagcccacaa |
| 192 | SOS1/ | agtagcgagcaggttcagttcgctagtaggaagctccagcgctacaccggcggcggcggtggcggcg |
| | SOS2 | gcggccgcggcggcacctcagggcagccagattccccaacaacgacctccggtccttccccttcccc |
| | mRNA | gcgagacctccgccccagctccgccccgcccggggctcttgcgtttcggagtcccaactacgcggag |
| | #3 | cgggcgccgcggccggagcgtccctccacacgtcctcccccagccgccagctccgccgcggggccag |
| | | accgggcccgggagtgcagccccggcgggcgggcggcggccgcggcagagggcagcccggggcggcg |
| | | ccggcgggccggggaggggcggggggcctggccccggggcggtggtgcgggatgccgccgcctccgg |
| | | gccgcgtccgttctccacggctggtacctgtgtcgggtgggtggccaggcgcgggcctcgcccccca |
| | | gccccctcgccagggctagcccggctgcgcggcgcccggagggggccgggccgtccggtggggccgc |
| | | ggccctgttccgcgctgcgagctcgccctctcgcggctccctggcccggccgccgccgcccctctcc |
| | | ccgcccagaggcgccccgggggcaccatgcaggcgcagcagctgccctacgagtttttcagcgaaga |
| | | gaacgcgcccaagtggcggggactactggtgcctgcgctgaaaaaggtccaggggcaagttcatcct |
| | | actctcgagtctaatgatgatgctcttcagtatgttgaagaattaattttgcaattattaaatatgc |
| | | tatgccaagctcagccccgaagtgcttcagatgtagaggaacgtgttcaaaaaagtttccctcatcc |
| | | aattgataaatgggcaatagctgatgcccaatcagctattgaaaagaggaagcgaagaaacccttta |
| | | tctctcccagtagaaaaaattcatcctttattaaaggaggtcctaggttataaaattgaccaccagg |
| | | tttctgtttacatagtagcagtcttagaatacatttctgcagacattttaaagctggttgggaatta |
| | | tgtaagaaatatacggcattatgaaattacaaaacaagatattaaagtggcaatgtgtgctgacaag |
| | | gtattgatggatatgtttcatcaagatgtagaagatattaatatattatctttaactgacgaagagc |
| | | cttccacctcaggagaacaaacttactatgatttggtaaaagcatttatggcagaaattcgacaata |
| | | tataagggaactaaatctaattataaaagtttttagagagccctttgtctccaattcaaaattgttt |
| | | tcagctaatgatgtagaaaatatatttagtcgcatagtagatatacatgaacttagtgtaaagttac |
| | | tgggccatatagaagatacagtagaaatgacagatgaaggcagtccccatccactagtaggaagctg |
| | | ctttgaagacttagcagaggaactggcatttgatccatatgaatcgtatgctcgagatattttgcga |
| | | cctggttttcatgatcgtttccttagtcagttatcaaagcctggggcagcactttatttgcagtcaa |
| | | taggcgaaggtttcaaagaagctgttcaatatgttttacccaggctgcttctggcccctgtttacca |
| | | ctgtctccattactttgaacttttgaagcagttagaagaaaaaagtgaagatcaagaagacaaggaa |
| | | tgtttaaaacaagcaataacagctttgcttaatgttcagagtggtatggaaaaaatatgttctaaaa |
| | | gtcttgcaaaacgaagactgagtgaatctgcatgtcggttttatagtcagcaaatgaaggggaaaca |
| | | actagcaatcaagaagatgaacgagattcagaagaatattgatggttgggagggaaaagacattgga |
| | | cagtgttgtaatgaatttataatggaaggaactcttacacgtgtaggagccaaacatgagagacaca |
| | | tatttctctttgatggcttaatgatttgctgtaaatcaaatcatgggcagccaagacttcctggtgc |
| | | tagcaatgcagaatatcgtcttaaagaaaagttttttatgcgaaaggtacaaattaatgataaagat |
| | | gacaccaatgaatacaagcatgcttttgaaataattttaaaagatgaaaatagtgttatattttctg |
| | | ccaagtcagctgaagagaaaaacaattggatggcagcattgatatctttacagtaccggagtacact |
| | | ggaaaggatgcttgatgtaacaatgctacaggaagagaaagaggagcagatgaggctgcctagtgct |
| | | gatgtttatagatttgcagagcctgactctgaagagaatattatatttgaagagaacatgcagccca |
| | | aggctggaattccaattatcaaagcaggaactgttattaaacttatagagaggcttacgtaccatat |
| | | gtacgcagatcccaattttgttcggacatttcttacaacatacagatccttttgcaaacctcaagaa |
| | | ctactgagtcttataatagaaaggtttgaaattccagagcctgagccaacagaagctgatcgcatag |
| | | ctatagagaatggagatcaacccttgagtgcagaactgaaaagatttagaaaagaatatatacagcc |
| | | tgtgcaactgcgagtattaaatgtatgtcggcactgggtagagcaccacttctatgattttgaaaga |
| | | gatgcatatcttttgcaacgaatggaagaatttattggaacagtaagaggtaaagcaatgaaaaaat |
| | | gggttgaatccatcactaaaataatccaaaggaaaaaaattgcaagagacaatggaccaggtcataa |
| | | tattacatttcagagttcacctcccacagttgagtggcatataagcagacctgggcacatagagact |
| | | tttgacctgctcaccttacacccaatagaaattgctcgacaactcactttacttgaatcagatctat |
| | | accgagctgtacagccatcagaattagttggaagtgtgtggacaaaagaagacaaagaaattaactc |
| | | tcctaatcttctgaaaatgattcgacataccaccaacctcactctgtggtttgagaaatgtattgta |
| | | gaaactgaaaatttagaagaaagagtagctgtggtgagtcgaattattgagattctacaagtctttc |
| | | aagagttgaacaactttaatggtgtccttgaggttgtcagtgctatgaattcatcacctgtttacag |
| | | actagaccacacatttgagcaaataccaagtcgccagaagaaaattttagaagaagctcatgaattg |
| | | agtgaagatcactataagaaatatttggcaaaactcaggtctattaatccaccatgtgtgcctttct |
| | | ttggaatttatctcactaatatcttgaaaacagaagaaggcaaccctgaggtcctaaaaagacatgg |
| | | aaaagagcttataaactttagcaaaaggaggaaagtagcagaaataacaggagagatccagcagtac |
| | | caaaatcagccttactgtttacgagtagaatcagatatcaaaaggttctttgaaaacttgaatccga |
| | | tgggaaatagcatggagaaggaatttacagattatcttttcaacaaatccctagaaatagaaccacg |
| | | aaaccctaagcctctcccaagatttccaaaaaaatatagctatcccctaaaatctcctggtgttcgt |
| | | ccatcaaacccaagaccaggtaccatgaggcatcccacacctctgcagcaggagccaaggaaaatta |
| | | gttatagtaggatccctgaaagtgaaacagaaagtacagcatctgcaccaaattctccaagaacacc |
| | | gttaacacctccgcctgcttctggtgcttccagtaccacagatgtttgcagtgtatttgattccgat |
| | | cattcgagcccttttcactcaagcaatgataccgtctttatccaagttactctgccccatggcccaa |
| | | gatctgcttctgtatcatctataagtttaaccaaaggcactgatgaagtgcctgtccctcctcctgt |
| | | tcctccacgaagacgaccagaatctgccccagcagaatcttcaccatctaagattatgtctaagcat |
| | | ttggacagtcccccagccattcctcctaggcaacccacatcaaaagcctattcaccacgatattcaa |
| | | tatcagaccggacctctatctcagaccctcctgaaagccctcccttattaccaccacgagaacctgt |
| | | gaggacacctgatgttttctcaagctcaccactacatctccaacctccccctttgggcaaaaaaagt |
| | | gaccatggcaatgccttcttcccaaacagcccttccccctttacaccacctcctcctcaaacacctt |
| | | ctcctcacggcacaagaaggcatctgccatcaccaccattgacacaagaagtggaccttcattccat |
| | | tgctgggccgcctgttcctccacgacaaagcacttctcaacatatccctaaactccctccaaaaact |
| | | tacaaaagggagcacacacacccatccatgcacagagatggaccaccactgttggagaatgcccatt |
| | | cttcctgagttcctctgtactgggatgtatattttcctagccccaaatccattgctggcaatggatg |
| | | cactgaatgtgccagcactgaggagttaaaatgagaactccaaacactaacgactcttcttcaagat |
| | | gcagtataagacaatgaattttaacctagatgtaattatacaatggaaatggtattccagtttagaa |
| | | tatggaaagaccgacctagaggaattggacaactgattgcacctgaaaatcataaagggactttttc |
| | | tggccaataggcaggagtcctctttttgtgaagtgatcttttgtgaagtgatcattaaagggatgga |
| | | aaacacagtctagtgtccagcaggcccacatgacagtttttgtaattcaaattatgcacttttaaaa |
| | | aaaaaaacttaaacagggatcttaatatcttcctttgttttcctttgctttactcttctactttaga |
| | | atattttcttaaaaatcactcaaaggactgtgaggaaaggctgtggtacctgaccttgttgaaatca |
| | | aggcccggcactgtactacaggcctgtttacagattattacggtgaactgaatgggtaccgaggctt |
| | | caccaaagaggtacttttttgttgttgttgttgttttaggaataattgtaccaattttaagagcatt |
| | | ccccccacctgtccccacacacccaaacaaaatgtggtggtgttgccttcaaaaaagagaagttttg |
| | | tgtcattaacatgacagaagaactttttaaaaaaaaataactgtcaactattctatttgcatttagg |
| | | agactgttcatctatgctagattgtcattttccctccttctcccacagaagtttactggtagtccat |
| | | gtcatggctcgtagctatccctctaaccataccatggaaatgcaggcacccaatgtgaaaaggagca |
| | | cttgctgggcatcactgacaccgctcatgttttacacatagttgagtaatcagcatatctagaatta |
| | | tcttgcattgcctaaatcatatgtatatagtgaatgttatataatatacctggcaggtctgttttaa |
| | | tttaattgaataaagatacaaatactttgtttggctggcatatattaaattattatatgaagaaaat |
| | | ggttgattcttatttcttttagttgaaaaacacacaaaaacactaaacataaaagcacgttttgtgg |
| | | tacgccttggtttctagttctggatatatgtattaatcttaaagaatgatctaaagtcacagcttgg |
| | | catataggaagatacttggttagaataggatgggtatcttttaagcagaactttgtaagtaagtttt |
| | | aagttcctaagcaacaagtatacagtttgttaaggtttatattgaacattctaccttgcttgaggtt |
| | | ttctgctttatcaaaactattcataatagaacatctacctgcatcattactatcataatatggatta |
| | | ttcataaagcgtatgtactgttcttctgagtctaacaaatactgtgactgaaatgcatgcaccttgt |
| | | tgaagagaaaacattttattaatctccactataatgtctgtgaaatgtgcagtattccatccttaga |
| | | gagtttatctggttcatcagcatctgcatgtatcctctgaataatgaagcaacatttcagcaattct |
| | | gttaaccacaccattagagacatttttagaagtgctgcaacatggtgggaacctagtactttaataa |
| | | tttcctatgatatttattagagcataaaatgtacatcacgatagtacagtatgtagtcctttaagac |
| | | cagaaggtatgttaactgcaccatcattaagcagtctatcctattttcttatttatttgttagaatt |
| | | gaatcctttctcaatatctgtaccattcctatgatggtagccattcttctctttataaaatatttcc |
| | | tctttgaaaagcgtcctaaaacattaatttcaaatcccctcttggatgcaaagtgtccctaagacca |
| | | tccattcaacagtatttatgtgagccaacttactagagccgctacaactaatcatgatagacagtcc |
| | | agaatctccctggactttatactgggccagcctcattgtccctaagatttctctttttatgtctcat |
| | | ggattacttctctttttaattgccccaggcacattccaaatgagaacctggtaaaagtcacattgga |
| | | cccccttcagtatgtcttcggctaatgtgttagccttcaataaggtgataaccccttttcaacctat |
| | | ctagtctataaagaggaaaaattcagatttggagttctaatcctatttctcccaaagggaactattc |
| | | ttctaccccatggttctaggagaccacttcactgtgcaacagttttacatgcattgtggatggagga |
| | | tatttttattctgaactgttacatttaggtaaaaatattttctgcagtggtaactgattagaaaaat |
| | | gccaattggatgcccttaggtggaggtgagaaaatggcatccttgccttcttctcaatatgaaacat |
| | | taactagttgacaaatttatccttgtagtaatgaaaatctatttaatcagggaccagaaatggctga |
| | | ggagataaatgcatcattacaaaattctgcttttgaatcctggacattacaagggggtaaatgcagc |
| | | atgactttttgttaaccacattccaaaatgtggaacatttcttttagaaatgaaaatatttcaaggc |
| | | tgatgtattttaagactacacattatcaggaaacatacattgagagttcgcttaattaaaggttgtt |
| | | gggcatcaaattatgtttagtaggttactattctctaacaactcaaggatgctttaatggatctgaa |
| | | tttgacaaagagcatgccacactaatactacagtcaacaacagcccagagaacaattactatgtcag |
| | | ctggaggctatattatgattctaaattcttaaaggtttttttccctccataaatcaaaaattacctt |
| | | atgtaaaccaaaaattagttggtatttatggtcatgatcttaattctcaagtttagcttaatcttgt |
| | | atttcattgtttgtcttctaatatgacagcttaaattcagatttttaagtgactcagcaaaatagga |
| | | ggagtgtcccaatttattagtgttgtacatattgaagaaaacctttttgttccttcagatttagaaa |
| | | gaaacagtttaaccatttatttcttggtattctgctgctgtgaataggtttacttattcttttaaca |
| | | tatattgtgtgactacgcaagtataggtcctgttgtattgcattaatctttaccagtaactaaacat |
| | | cacaaggttaatattggtttggctgaaagaattatgcagtaaagttatttataagggaacatgatga |
| | | ctttattcaatatttttcttctttgaaacatctcattactaacttttaagattatttcataatccct |
| | | tatacatgagccaatgaaatattttgagctctacttaagaagcatgaagtctatattataaatctaa |
| | | acaacaaaagcacttgtaacttgtttagtaaattccatgccttattttccatttttgacaccgtaaa |
| | | gtgcattttctgtcgactgtgagcaaatttcccttttgcctttaatgaaaatagtgcattaagtagc |
| | | caattgctgcagaattataaagcaaattagctaacagtgggtcacattggactaccactcaaagaaa |
| | | caagatgcataacagatgatggaaggtacatgtggtgtggcttagggagagcgcacctgtgccagat |
| | | gcatgccactttcagtagctgccaaatgtgcctttaattaagaacatctctaatttttttcttcaga |
| | | tcaagtcagcatttggggtcgggatttgggcagggaactttggggtgcaaatatctggtgaaaccac |
| | | cagagaatttgtttgtcaaagtgacatgtataattttagttgagacgtgtttgtgtatatatgtgta |
| | | tttgcctttgtttagtagcacggttatttgctaaaataattgtcaaaaaattgctctgtcggtcggt |
| | | ctttgatgtaagaacaaagtgtggtctatacatctaatatggcctttgtacctaaccatgttcatag |
| | | gtaatctttgtactctgtgtgcagcagtatttggtttgcatttaaagtgaaaataacggctgttatt |
| | | tttctggcattactaagataaactgaaaaataataaagaaaaatgccttacatagcccacaa |
| 193 | SOS1/ | agaagcgggccagcgccgccgggaaaggaggtcgccgcccggggtcgcccgggcttgcgggtcgccg |
| | SOS2 | cgcttcctctgcgggcgctgcgggaggcgagggccgcctcggcggaggaagccgaccccggggtctc |
| | mRNA | cgccagcccgccggcgccgcctccgagccgcctcggccgtagcctcggcccgggcgggaggcggcgg |
| | #4 | ccgccggcgaggcgaggcggcccgcgccctgcctgtcaggccaccggcccggcccgcgggctgcccc |
| | | cgatgcggaggcgctgtcgccggggccatgcagcaggcgccgcagccttacgagttcttcagcgagg |
| | | agaacagtccgaaatggcggggactgttggtctcggccctgcggaaggttcaggaacaagtgcatcc |
| | | cactctctcagctaatgaagagtctctctattatattgaagagctgatttttcagctgcttaataaa |
| | | ttatgcatggcccagccaaggactgttcaagatgtagaggagcgagttcagaagacctttcctcacc |
| | | caattgataaatgggccattgctgatgcacaatctgctatagaaaaacgaaaacgaagaaatcctct |
| | | tttactgcctgtggacaaaatccatccttcgttgaaggaagtattagggtacaaagtggactaccat |
| | | gtatccctatatattgtggctgtactagagtatatctcagctgatattttaaaattggctggtaatt |
| | | atgtttttaatatccggcattatgaaatatctcagcaggacattaaagtgtcaatgtgtgcggataa |
| | | ggttttgatggacatgtttgatcaggatgacataggtttggtttctctctgtgaagatgaacctagt |
| | | tcttctggtgaattaaactactatgatcttgtcagaactgaaatcgcagaagaaagacagtatctac |
| | | gggaattaaatatgatcataaaagtgtttcgagaagcctttctttctgatagaaagctgtttaaacc |
| | | ttctgatatcgaaaagatttttagtaacatttcagatatacatgaattgactgtgaaacttttaggt |
| | | ttgattgaagacacagttgaaatgactgatgaaagcagtcctcatcccttagctggcagctgttttg |
| | | aagatttggcagaagagcaagcatttgatccttatgaaacattatcacaggacattctttcaccaga |
| | | gtttcatgaacatttcaataaattgatggccagacctgcagttgctctacactttcagtccattgct |
| | | gatggttttaaagaggcagttcgttatgtccttccacgtcttatgctggtgccagtgtatcactgtt |
| | | ggcactactttgagttactaaagcaattgaaagcatgtagtgaagaacaagaagacagagaatgttt |
| | | gaaccaagctattactgctctcatgaatctccaaggtagcatggaccgaatttacaagcagtattca |
| | | cctagacgtcgacctggagatcctgtttgccctttttatagtcaccaattaagaagcaaacacctgg |
| | | ctatcaaaaaaatgaatgaaattcagaaaaatatcgatggatgggaaggcaaagatattggacagtg |
| | | ttgtaatgaattcattatggagggaccattgacaagaatcggtgccaaacatgaacggcatattttt |
| | | ctgtttgatggcttaatgatcagttgtaaacctaatcatggccagactcggcttccaggttacagta |
| | | gtgcagaatacaggttaaaagaaaaatttgtcatgaggaaaatacaaatttgtgataaagaagatac |
| | | ttgtgagcacaagcatgcatttgaattagtatccaaagatgagaacagcataatatttgctgctaag |
| | | tctgctgaagaaaaaaacaactggatggcagcccttatttctcttcattatcgtagtactctagatc |
| | | gaatgttagattcagtattattgaaagaagaaaatgagcaaccactgagattaccaagtcctgaagt |
| | | atatcgttttgtagtaaaagactctgaggaaaacattgtttttgaagacaacttgcaaagtagaagt |
| | | ggcatccccattattaaaggaggaactgtagtgaaattaattgaaaggttaacatatcatatgtatg |
| | | cagatcccaattttgttcgtacttttcttaccacatatcgttcattttgtaaaccacaggaattgct |
| | | gagcttactgattgaacggtttgaaattccagagccagaacctactgacgcagacaaattggcaata |
| | | gagaaaggcgagcagccaatcagtgcagaccttaaaagatttcgcaaggaatatgtccaaccagtac |
| | | aacttaggatcttaaatgtatttcggcattgggttgaacatcatttttatgactttgaaagagactt |
| | | ggaattgcttgaaagactagaatccttcatttcaagtgtaagagggaaagctatgaaaaaatgggta |
| | | gagtcaattgctaagatcatcaggaggaagaagcaagctcaggcaaacggagtaagccataatatta |
| | | cctttgaaagtccacctccaccaattgaatggcatatcagcaaaccaggacagtttgaaacatttga |
| | | tctcatgacacttcatccaatagaaattgcacgtcagctgacacttttggagtctgatctttacagg |
| | | aaagttcaaccgtctgaacttgtagggagtgtgtggaccaaagaagataaagaaataaattctccaa |
| | | atttattaaaaatgattcgccataccacaaatctcaccctctggtttgaaaaatgcattgtggaagc |
| | | agaaaattttgaagaacgggtggcagtactaagtagaattatagaaattctgcaagtttttcaagat |
| | | ttgaataatttcaatggcgtattggagatagtcagtgcagtaaattcagtgtcagtatacagactag |
| | | accatacctttgaggcactgcaggaaaggaaaaggaaaattttggacgaagctgtggaattaagtca |
| | | agatcactttaaaaaatacctagtaaaacttaagtcaatcaatccaccttgtgtgcctttttttgga |
| | | atatatttaacaaatattctgaagaccgaagaagggaataatgattttttaaaaaagaaagggaaag |
| | | atttaatcaatttcagtaagaggaggaaagtagctgaaattactggagaaattcagcagtatcagaa |
| | | tcagccttactgtttacggatagaaccagatatgaggagattctttgaaaaccttaaccccatggga |
| | | agtgcatctgaaaaagagtttacagattatttgttcaacaagtcactagaaattgaacctcgaaact |
| | | gcaaacagccacctcgatttcctaggaaatcaactttttccttaaaatctcctggaataaggcctaa |
| | | cacaggccgacatggctctacctcaggtactttacgaggtcacccaacaccattagaaagagaacca |
| | | tgtaaaataagctttagtcggattgctgaaactgagctggaatcaacagtgtcagcaccaacctctc |
| | | caaatacaccatctactccaccagtatctgcttcttcagaccttagtgtatttttagatgtggatct |
| | | caacagctcctgtggcagcaatagcatctttgctccagtgcttttgccacattcaaagtctttcttt |
| | | agttcatgtggtagtttacataaactaagtgaagagcccctgattcctcctcctcttcctcctcgaa |
| | | aaaagtttgatcatgatgcttcaaattccaagggaaatatgaaatctgatgatgatcctcctgctat |
| | | tccaccgagacagcctcctcctccaaaggtaaaacccagagttcctgttcctactggtgcatttgat |
| | | gggcctctgcatagtccacctccgccaccaccaagagatcctcttcctgatacccctccaccagttc |
| | | cccttcggcctccagaacactttataaactgtccatttaatcttcagccacctccactggggcatct |
| | | tcacagagattcagactggctcagagacattagtacgtgtccaaattcgccaagcactcctcctagc |
| | | acaccctctccaagggtaccgcgtcgatgctatgtgctcagttctagtcagaataatcttgctcatc |
| | | ctccagctccccctgttccaccaaggcagaattcaagccctcatctgccaaaactgccaccaaagac |
| | | ttacaaacgggagctttcgcaccccccattgtacagactgcctttgctagaaaatgcagaaactccc |
| | | caatgaccttagccatatgtagtcattgacactggaatggtatttgtaaagttttttttttaattta |
| | | ttcaaaaaaagacatagtattttagtactttttacaaataatgctcttacaaaaatgctactgatca |
| | | aataagcttttaagaattggaaaaataaaaatacaaaagtccttcaccattatcctcaggtgatcag |
| | | tagcattgccttgtcttggatcctcagtgctgccaaaaggccagtataaagaatttatatttgcact |
| | | gtaaactctgcaaaaatatggtttaaagtgacatgattgcactgaaaagggatagtgcttttgtgaa |
| | | atttttcaaatttgagtaatagatgcctttttaaggcagtgaatttacacaaatatgggaggggtat |
| | | atggtgttactgatttttaaacctctttgaccatcttctagtttttacttctagtttttacatctag |
| | | gagaattgtgaataacaccaactgttcttaaactctttaatgccatgtcttaaatgccggtatttgc |
| | | tgctgaagacaaaaatgaaaagtaggatgaaaataatagaatggcactgtaagtgtttattattttg |
| | | tcaaaatgtaaacaaagactacataacccaatgatggagggaaaaagggcatgtatctcattcagat |
| | | gtgccttttgtttttgcagactatgacgtctttagctaatgaattgcctattgttatggaaaacagt |
| | | taatatgccatgtatgtacagttttgtttatattgtatatttaaagatactgctaataacctatata |
| | | aatttaagtgacttgaggcctataatacaatctgctactttactaattcataaattcaaaaaaaatt |
| | | ctatggcataggaaccaactgccttgccttcaagacctagtaactttctctataaatctcgtgttaa |
| | | ctgaaattttttttaaatatattttttagattggtaatatttaaaccagcaaatacttaaagcttta |
| | | ttaaacattttaatcagataagtgagtaaagcttttatttgccatttggatgccttcgttcaaagtg |
| | | atagagtgttttgctgatagtgctgtagcagcagttgtaaagtagccaaaagccacgttgtttattt |
| | | actggtttgtggccttttactgtgctttgtatcagagttcttaacaagattaataaatcaccccagt |
| | | cttaatttttaaaa |

What is claimed is:

1. An antisense oligonucleotide that inhibits the expression of son of sevenless 1 (SOS1) or son of sevenless 2 (SOS2) mRNA, wherein the antisense oligonucleotide comprises 10 to 30 linked nucleotides and has a sequence that is complementary to SOS1 or SOS2 mRNA, and wherein the oligonucleotide has at least 8 contiguous nucleotides of any one of SEQ ID NOS: 1 to 189.

2. The antisense oligonucleotide of claim 1, wherein the oligonucleotide is at least 12 nucleotides in length.

3. The antisense oligonucleotide of claim 2, wherein the oligonucleotide is at least 14 nucleotides in length.

4. The antisense oligonucleotide of claim 2, wherein the oligonucleotide is from 10 to 24 nucleotides in length, or 10 to 16 nucleotides in length, or 12 to 16 nucleotides in length.

5. The antisense oligonucleotide of claim 4, wherein the oligonucleotide is 12, 13, 14, 15, or 16 nucleotides in length.

6. The antisense oligonucleotide of claim 5, wherein the oligonucleotide is 14 nucleotides in length.

7. The antisense oligonucleotide of any one of claims 1 to 6, wherein the oligonucleotide comprises at least 8, at least 10, or at least 12 contiguous nucleobases of any one of SEQ ID NOs: 1 to 189.

8. The antisense oligonucleotide of claim 7, wherein the oligonucleotide comprises or consists of a nucleobase sequence of any one of SEQ ID Nos: 1 to 189.

9. The antisense oligonucleotide of any one of claims 1 to 8, wherein the oligonucleotide has a nucleobase sequence and structure selected from Table 4.

10. The antisense oligonucleotide of claim 8 or 9, wherein the oligonucleotide has a nucleobase sequence selected from SEQ ID NOs: 2, 3, 5, 6, 7, 10, 11, 15, 16, 17, 18, 19, 20, 21, 23, 26, 27, 31, 41, 42, 43, 47, 48, 49, 51, 56, 61, 63, 64, 65, 66, 73, 74, 80, 83, 84, 85, 86, 87, 88, 94, 95, 96, 97, 98, 101, 107, 111, 116, 119, 135, 136, 138, 139, 143, 144, 150, 157, 158, 161, 164, 172, 178, 179, 181 and 187.

11. The antisense oligonucleotide of any one of claims 1 to 10, wherein the antisense oligonucleotide has a stretch of at least 6 DNA nucleotides sufficient to recruit RNaseH.

12. The antisense oligonucleotide of claim 11, wherein the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides.

13. The antisense oligonucleotide of claim 12, wherein the 5′ and 3′ segments are each 3 nucleotides in length, and the 5′ and 3′ segments flank an internal sequence of 8 DNA nucleotides.

14. The antisense oligonucleotide of claim 13, wherein one or more nucleotides of the 5′ segment and the 3′ segment comprise 2′-O substituents, optionally where all of the nucleotides of the 5′ segment and the 3′ segment comprise 2′-O substituents.

15. The antisense oligonucleotide of claim 14, wherein the 2′-O substituents are independently selected from 2′-O methyl, 2′-O ethyl, 2′-O methoxyethyl (MOE), and a bridged nucleotide having a 2′ to 4′ bridge.

16. The antisense oligonucleotide of claim 15, wherein the bridged nucleotide has a methylene bridge (LNA) or a constrained ethyl bridge (cEt).

17. The antisense oligonucleotide of any one of claims 1 to 16, wherein the antisense oligonucleotide has a modified backbone.

18. The antisense oligonucleotide of claim 17, wherein the antisense oligonucleotide comprises one or more phosphorothioate or phosphorodithioate nucleotides.

19. The antisense oligonucleotide of claim 18, wherein the oligonucleotide is fully phosphorothioate or phosphorodithioate linked.

20. The antisense oligonucleotide of claim 19, wherein the oligonucleotide is fully phosphorothioate linked.

21. The antisense oligonucleotide of any one of claims 1 to 20, wherein cytidine nucleobases in the antisense oligonucleotide are 5-methyl cytidine.

22. The antisense oligonucleotide of any one of claims 1 to 21, further comprising a cell penetrating moiety.

23. The antisense oligonucleotide of claim 22, wherein the cell penetrating moiety is conjugated directly or indirectly at the 3′ end of the oligonucleotide, optionally though a linker.

24. The antisense oligonucleotide of claim 22 or claim 23, wherein the moiety comprises a sterol conjugate or fatty acid conjugate, which is optionally cholesteryl, palmitoyl, or stearyl conjugate.

25. The antisense oligonucleotide of any one of claims 1 to 24, wherein the composition does not comprise any encapsulation or transfection reagent.

26. The antisense oligonucleotide of any one of claims 1 to 25, wherein the antisense oligonucleotide is encapsulated in a particle.

27. The antisense oligonucleotide of claim 26, wherein the particle is a liposome, polymeric nanoparticle, or lipid nanoparticle.

28. The antisense oligonucleotide of any one of claims 1 to 27, wherein the oligonucleotide is formulated for parenteral administration.

29. A pharmaceutical composition comprising an antisense oligonucleotide of any one of claims 1 to 28, and a pharmaceutically acceptable carrier or vehicle.

30. A method for treating a subject having a condition associated with aberrant expression or activity of SOS1 or SOS2, comprising administering an effective amount of the composition of any one of claims 1 to 28, or the composition of claim 29, to the subject.

31. The method of claim 30, wherein the subject has Noonan syndrome.

32. The method of claim 30, wherein the subject has hereditary gingival fibromatosis type 1.

33. The method of claim 30, wherein the subject has a malignancy associated with aberrant expression of SOS1 or SOS2.

34. The method of claim 33, wherein the malignancy associated with aberrant expression of SOS1 or SOS2 is non-metastatic.

35. The method of claim 33, wherein the malignancy associated with aberrant expression of SOS1 or SOS2 is metastatic.

36. The method of any one of claims 33 to 35, wherein the malignancy is a carcinoma.

37. The method of claim 36, wherein the malignancy is breast cancer, cervical cancer, pancreatic cancer, squamous cell carcinoma, head and neck cancer, thyroid cancer, gastric cancer, colon cancer, or liver cancer.

38. The method of claim 36 or 37, wherein the malignancy is cervical squamous cell carcinoma, endocervical adenocarcinoma, cholangiocarcinoma, esophageal carcinoma, colon adenocarcinoma, oral squamous cell carcinoma, head and neck squamous cell carcinoma, lung adenocarcinoma, lung squamous cell carcinoma, non-small cell lung cancer, liver hepatocellular carcinoma, lung squamous cell carcinoma, rectum adenocarcinoma, stomach adenocarcinoma, thyroid carcinoma, or pancreatic adenocarcinoma.

39. The method of any one of claims 30 to 38, wherein the composition is administered parenterally.


SEQUENCE LISTING (195 sequences)

SEQ ID NO: 1 - DNA, 14 bp, synthetic construct
  source 1..14: mol_type = other DNA
atttcataatgccg

SEQ ID NO: 2 - DNA, 14 bp, synthetic construct
  source 1..14: mol_type = other DNA
tgcacttgttcctg

SEQ ID NO: 3 - DNA, 14 bp, synthetic construct
  source 1..14: mol_type = other DNA
gagtgggatgcact

SEQ ID NO: 4 - DNA, 14 bp, synthetic construct
  source 1..14: mol_type = other DNA
tagcagattgtgca

SEQ ID NO: 5 - DNA, 14 bp, synthetic construct
  source 1..14: mol_type = other DNA
tcgtttttctatag

SEQ ID NO: 6 - DNA, 14 bp, synthetic construct
  source 1..14: mol_type = other DNA
cgttttcgtttttc

SEQ ID NO: 7 - DNA, 14 bp, synthetic construct
  source 1..14: mol_type = other DNA
cttcgttttcgttt

SEQ ID NO: 8 - DNA, 14 bp, synthetic construct
  source 1..14: mol_type = other DNA
ttcttcgttttcgt

SEQ ID NO: 9 - DNA, 14 bp, synthetic construct
  source 1..14: mol_type = other DNA
atggtagtccactt

SEQ ID NO: 10 - DNA, 14 bp, synthetic construct
  source 1..14: mol_type = other DNA
tacatggtagtcca

SEQ ID NO: 11 - DNA, 14 bp, synthetic construct
  source 1..14: mol_type = other DNA
gatacatggtagtc

SEQ ID NO: 12 - DNA, 14 bp, synthetic construct
  source 1..14: mol_type = other DNA
gacactttaatgtc

SEQ ID NO: 13 - DNA, 14 bp, synthetic construct
  source 1..14: mol_type = other DNA
gagagaaaccaaac

SEQ ID NO: 14 - DNA, 14 bp, synthetic construct
  source 1..14: mol_type = other DNA
atagtagtttaatt

SEQ ID NO: 15 - skipped

SEQ ID NO: 16 - DNA, 14 bp, synthetic construct
  source 1..14: mol_type = other DNA
gcgatttcagttct

SEQ ID NO: 17 - DNA, 14 bp, synthetic construct
  source 1..14: mol_type = other DNA
atatttaattcccg

SEQ ID NO: 18 - DNA, 14 bp, synthetic construct
  source 1..14: mol_type = other DNA
acacttttatgatc

SEQ ID NO: 19 - DNA, 14 bp, synthetic construct
  source 1..14: mol_type = other DNA
tcgaaacactttta

SEQ ID NO: 20 - DNA, 14 bp, synthetic construct
  source 1..14: mol_type = other DNA
ttctcgaaacactt

SEQ ID NO: 21 - DNA, 14 bp, synthetic construct
  source 1..14: mol_type = other DNA
ttcatgtatatctg

SEQ ID NO: 22 - DNA, 14 bp, synthetic construct
  source 1..14: mol_type = other DNA
gtcaattcatgtat

SEQ ID NO: 23 - DNA, 14 bp, synthetic construct
  source 1..14: mol_type = other DNA
cttcaatcaaacct

SEQ ID NO: 24 - DNA, 14 bp, synthetic construct
  source 1..14: mol_type = other DNA
gtgataatgtttca

SEQ ID NO: 25 - DNA, 14 bp, synthetic construct
  source 1..14: mol_type = other DNA
gtgtagagcaactg

SEQ ID NO: 26 - DNA, 14 bp, synthetic construct
  source 1..14: mol_type = other DNA
aagtgtagagcaac

SEQ ID NO: 27 - DNA, 14 bp, synthetic construct
  source 1..14: mol_type = other DNA
ctgaaagtgtagag

SEQ ID NO: 28 - DNA, 14 bp, synthetic construct
  source 1..14: mol_type = other DNA
agcataagacgtgg

SEQ ID NO: 29 - DNA, 14 bp, synthetic construct
  source 1..14: mol_type = other DNA
acagtgatacactg

SEQ ID NO: 30 - DNA, 14 bp, synthetic construct
  source 1..14: mol_type = other DNA
gtaactcaaagtag

SEQ ID NO: 31 - DNA, 14 bp, synthetic construct
  source 1..14: mol_type = other DNA
ttgttcttcactac

SEQ ID NO: 32 - DNA, 14 bp, synthetic construct
  source 1..14: mol_type = other DNA
aatagcttggttca

SEQ ID NO: 33 - DNA, 14 bp, synthetic construct
  source 1..14: mol_type = other DNA
agtaatagcttggt

SEQ ID NO: 34 - DNA, 14 bp, synthetic construct
  source 1..14: mol_type = other DNA
cttgtaaattcggt

SEQ ID NO: 35 - DNA, 14 bp, synthetic construct
  source 1..14: mol_type = other DNA
cgacgtctaggtga

SEQ ID NO: 36 - DNA, 14 bp, synthetic construct
  source 1..14: mol_type = other DNA
taattggtgactat

SEQ ID NO: 37 - DNA, 14 bp, synthetic construct
  source 1..14: mol_type = other DNA
cttaattggtgact

SEQ ID NO: 38 - DNA, 14 bp, synthetic construct
  source 1..14: mol_type = other DNA
cccatccatcgata

SEQ ID NO: 39 - DNA, 14 bp, synthetic construct
  source 1..14: mol_type = other DNA
aattcattacaaca

SEQ ID NO: 40 - DNA, 14 bp, synthetic construct
  source 1..14: mol_type = other DNA
cgattcttgtcaat

SEQ ID NO: 41 - DNA, 14 bp, synthetic construct
  source 1..14: mol_type = other DNA
caccgattcttgtc

SEQ ID NO: 42 - DNA, 14 bp, synthetic construct
  source 1..14: mol_type = other DNA
ttggcaccgattct

SEQ ID NO: 43 - DNA, 14 bp, synthetic construct
  source 1..14: mol_type = other DNA
atgtttggcaccga

SEQ ID NO: 44 - DNA, 14 bp, synthetic construct
  source 1..14: mol_type = other DNA
gccgttcatgtttg

SEQ ID NO: 45 - DNA, 14 bp, synthetic construct
  source 1..14: mol_type = other DNA
tgattaggtttaca

SEQ ID NO: 46 - DNA, 14 bp, synthetic construct
  source 1..14: mol_type = other DNA
cactactgtaacct

SEQ ID NO: 47 - DNA, 14 bp, synthetic construct
  source 1..14: mol_type = other DNA
catgacaaattttt

SEQ ID NO: 48 - DNA, 14 bp, synthetic construct
  source 1..14: mol_type = other DNA
gctcacaagtatct

SEQ ID NO: 49 - DNA, 14 bp, synthetic construct
  source 1..14: mol_type = other DNA
atgcatgcttgtgc

SEQ ID NO: 50 - DNA, 14 bp, synthetic construct
  source 1..14: mol_type = other DNA
tcaaatgcatgctt

SEQ ID NO: 51 - DNA, 14 bp, synthetic construct
  source 1..14: mol_type = other DNA
gcagcaaatattat

SEQ ID NO: 52 - DNA, 14 bp, synthetic construct
  source 1..14: mol_type = other DNA
ttcgatctagagta

SEQ ID NO: 53 - DNA, 14 bp, synthetic construct
  source 1..14: mol_type = other DNA
aacattcgatctag

SEQ ID NO: 54 - DNA, 14 bp, synthetic construct
  source 1..14: mol_type = other DNA
ggtaatctcagtgg

SEQ ID NO: 55 - DNA, 14 bp, synthetic construct
  source 1..14: mol_type = other DNA
caaaacgatatact

SEQ ID NO: 56 - DNA, 14 bp, synthetic construct
  source 1..14: mol_type = other DNA
ctacaaaacgatat

SEQ ID NO: 57 - DNA, 14 bp, synthetic construct
  source 1..14: mol_type = other DNA
acaatgttttcctc

SEQ ID NO: 58 - DNA, 14 bp, synthetic construct
  source 1..14: mol_type = other DNA
tgcaagttgtcttc

SEQ ID NO: 59 - DNA, 14 bp, synthetic construct
  source 1..14: mol_type = other DNA
cctttaataatggg

SEQ ID NO: 60 - DNA, 14 bp, synthetic construct
  source 1..14: mol_type = other DNA
tgttaacctttcaa

SEQ ID NO: 61 - DNA, 14 bp, synthetic construct
  source 1..14: mol_type = other DNA
attgggatctgcat

SEQ ID NO: 62 - DNA, 14 bp, synthetic construct
  source 1..14: mol_type = other DNA
acgatatgtggtaa

SEQ ID NO: 63 - DNA, 14 bp, synthetic construct
  source 1..14: mol_type = other DNA
gctcagcaattcct

SEQ ID NO: 64 - DNA, 14 bp, synthetic construct
  source 1..14: mol_type = other DNA
gaatttcaaaccgt

SEQ ID NO: 65 - DNA, 14 bp, synthetic construct
  source 1..14: mol_type = other DNA
cgtcagtaggttct

SEQ ID NO: 66 - DNA, 14 bp, synthetic construct
  source 1..14: mol_type = other DNA
aggtctgcactgat

SEQ ID NO: 67 - DNA, 14 bp, synthetic construct
  source 1..14: mol_type = other DNA
tcttttaaggtctg

SEQ ID NO: 68 - DNA, 14 bp, synthetic construct
  source 1..14: mol_type = other DNA
aatcttttaaggtc

SEQ ID NO: 69 - DNA, 14 bp, synthetic construct
  source 1..14: mol_type = other DNA
gcgaaatcttttaa

SEQ ID NO: 70 - DNA, 14 bp, synthetic construct
  source 1..14: mol_type = other DNA
cctaagttgtactg

SEQ ID NO: 71 - DNA, 14 bp, synthetic construct
  source 1..14: mol_type = other DNA
gatcctaagttgta

SEQ ID NO: 72 - DNA, 14 bp, synthetic construct
  source 1..14: mol_type = other DNA
ccctcttacacttg

SEQ ID NO: 73 - DNA, 14 bp, synthetic construct
  source 1..14: mol_type = other DNA
gactctacccattt

SEQ ID NO: 74 - DNA, 14 bp, synthetic construct
  source 1..14: mol_type = other DNA
agcaattgactcta

SEQ ID NO: 75 - DNA, 14 bp, synthetic construct
  source 1..14: mol_type = other DNA
ttagcaattgactc

SEQ ID NO: 76 - DNA, 14 bp, synthetic construct
  source 1..14: mol_type = other DNA
atcttagcaattga

SEQ ID NO: 77 - DNA, 14 bp, synthetic construct
  source 1..14: mol_type = other DNA
atgatcttagcaat

SEQ ID NO: 78 - DNA, 14 bp, synthetic construct
  source 1..14: mol_type = other DNA
ctgatgatcttagc

SEQ ID NO: 79 - DNA, 14 bp, synthetic construct
  source 1..14: mol_type = other DNA
tggcttactccgtt

SEQ ID NO: 80 - DNA, 14 bp, synthetic construct
  source 1..14: mol_type = other DNA
taatattatggctt

SEQ ID NO: 81 - DNA, 14 bp, synthetic construct
  source 1..14: mol_type = other DNA
cattcaattggtgg

SEQ ID NO: 82 - DNA, 14 bp, synthetic construct
  source 1..14: mol_type = other DNA
atgaagtgtcatga

SEQ ID NO: 83 - DNA, 14 bp, synthetic construct
  source 1..14: mol_type = other DNA
ctgacgtgcaattt

SEQ ID NO: 84 - DNA, 14 bp, synthetic construct
  source 1..14: mol_type = other DNA
tcagctgacgtgca

SEQ ID NO: 85 - DNA, 14 bp, synthetic construct
  source 1..14: mol_type = other DNA
tgtcagctgacgtg

SEQ ID NO: 86 - DNA, 14 bp, synthetic construct
  source 1..14: mol_type = other DNA
gacggttgaacttt

SEQ ID NO: 87 - DNA, 14 bp, synthetic construct
  source 1..14: mol_type = other DNA
acaagttcagacgg

SEQ ID NO: 88 - DNA, 14 bp, synthetic construct
  source 1..14: mol_type = other DNA
atggcgaatcattt

SEQ ID NO: 89 - skipped

SEQ ID NO: 90 - DNA, 14 bp, synthetic construct
  source 1..14: mol_type = other DNA
tggtatggcgaatc

SEQ ID NO: 91 - DNA, 14 bp, synthetic construct
  source 1..14: mol_type = other DNA
tgtggtatggcgaa

SEQ ID NO: 92 - DNA, 14 bp, synthetic construct
  source 1..14: mol_type = other DNA
agatttgtggtatg

SEQ ID NO: 93 - DNA, 14 bp, synthetic construct
  source 1..14: mol_type = other DNA
gccacccgttcttc

SEQ ID NO: 94 - DNA, 14 bp, synthetic construct
  source 1..14: mol_type = other DNA
gcagaatttctata

SEQ ID NO: 95 - DNA, 14 bp, synthetic construct
  source 1..14: mol_type = other DNA
gccattgaaattat

SEQ ID NO: 96 - DNA, 14 bp, synthetic construct
  source 1..14: mol_type = other DNA
gaatttactgcact

SEQ ID NO: 97 - DNA, 14 bp, synthetic construct
  source 1..14: mol_type = other DNA
gacactgaatttac

SEQ ID NO: 98 - DNA, 14 bp, synthetic construct
  source 1..14: mol_type = other DNA
tgacttaattccac

SEQ ID NO: 99 - DNA, 14 bp, synthetic construct
  source 1..14: mol_type = other DNA
gttttactaggtat

SEQ ID NO: 100 - DNA, 14 bp, synthetic construct
  source 1..14: mol_type = other DNA
aagttttactaggt

SEQ ID NO: 101 - DNA, 14 bp, synthetic construct
  source 1..14: mol_type = other DNA
ttcttcggtcttca

SEQ ID NO: 102 - DNA, 14 bp, synthetic construct
  source 1..14: mol_type = other DNA
tcccttcttcggtc

SEQ ID NO: 103 - DNA, 14 bp, synthetic construct
  source 1..14: mol_type = other DNA
atcattattccctt

SEQ ID NO: 104 - DNA, 14 bp, synthetic construct
  source 1..14: mol_type = other DNA
aatctttccctttc

SEQ ID NO: 105 - DNA, 14 bp, synthetic construct
  source 1..14: mol_type = other DNA
ccgtaaacagtaag

SEQ ID NO: 106 - DNA, 14 bp, synthetic construct
  source 1..14: mol_type = other DNA
tctatccgtaaaca

SEQ ID NO: 107 - DNA, 14 bp, synthetic construct
  source 1..14: mol_type = other DNA
ctggttctatccgt

SEQ ID NO: 108 - DNA, 14 bp, synthetic construct
  source 1..14: mol_type = other DNA
cctcatatctggtt

SEQ ID NO: 109 - DNA, 14 bp, synthetic construct
  source 1..14: mol_type = other DNA
gggttaaggttttc

SEQ ID NO: 110 - DNA, 14 bp, synthetic construct
  source 1..14: mol_type = other DNA
tcccatggggttaa

SEQ ID NO: 111 - DNA, 14 bp, synthetic construct
  source 1..14: mol_type = other DNA
tttcgaggttcaat

SEQ ID NO: 112 - DNA, 14 bp, synthetic construct
  source 1..14: mol_type = other DNA
ggtggctgtttgca

SEQ ID NO: 113 - DNA, 14 bp, synthetic construct
  source 1..14: mol_type = other DNA
tcgaggtggctgtt

SEQ ID NO: 114 - DNA, 14 bp, synthetic construct
  source 1..14: mol_type = other DNA
gttgatttcctagg

SEQ ID NO: 115 - DNA, 14 bp, synthetic construct
  source 1..14: mol_type = other DNA
tgttaggccttatt

SEQ ID NO: 116 - DNA, 14 bp, synthetic construct
  source 1..14: mol_type = other DNA
tgtcggcctgtgtt

SEQ ID NO: 117 - DNA, 14 bp, synthetic construct
  source 1..14: mol_type = other DNA
catgtcggcctgtg

SEQ ID NO: 118 - DNA, 14 bp, synthetic construct
  source 1..14: mol_type = other DNA
gtagagccatgtcg

SEQ ID NO: 119 - DNA, 14 bp, synthetic construct
  source 1..14: mol_type = other DNA
gttgggtgacctcg

SEQ ID NO: 120 - DNA, 14 bp, synthetic construct
  source 1..14: mol_type = other DNA
ctctttctaatggt

SEQ ID NO: 121 - DNA, 14 bp, synthetic construct
  source 1..14: mol_type = other DNA
ccgactaaagctta

SEQ ID NO: 122 - DNA, 14 bp, synthetic construct
  source 1..14: mol_type = other DNA
caatccgactaaag

SEQ ID NO: 123 - DNA, 14 bp, synthetic construct
  source 1..14: mol_type = other DNA
agcaatccgactaa

SEQ ID NO: 124 - DNA, 14 bp, synthetic construct
  source 1..14: mol_type = other DNA
ttcagcaatccgac

SEQ ID NO: 125 - DNA, 14 bp, synthetic construct
  source 1..14: mol_type = other DNA
tgttgattccagct

SEQ ID NO: 126 - DNA, 14 bp, synthetic construct
  source 1..14: mol_type = other DNA
ctgacactgttgat

SEQ ID NO: 127 - DNA, 14 bp, synthetic construct
  source 1..14: mol_type = other DNA
tacactaaggtctg

SEQ ID NO: 128 - DNA, 14 bp, synthetic construct
  source 1..14: mol_type = other DNA
ggagcaaagatgct

SEQ ID NO: 129 - DNA, 14 bp, synthetic construct
  source 1..14: mol_type = other DNA
gggctcttcactta

SEQ ID NO: 130 - DNA, 14 bp, synthetic construct
  source 1..14: mol_type = other DNA
aaacttttttcgag

SEQ ID NO: 131 - DNA, 14 bp, synthetic construct
  source 1..14: mol_type = other DNA
ggtggaatagcagg

SEQ ID NO: 132 - DNA, 14 bp, synthetic construct
  source 1..14: mol_type = other DNA
gaggctgtctcggt

SEQ ID NO: 133 - DNA, 14 bp, synthetic construct
  source 1..14: mol_type = other DNA
ggtggactatgcag

SEQ ID NO: 134 - DNA, 14 bp, synthetic construct
  source 1..14: mol_type = other DNA
gtggctgaagatta

SEQ ID NO: 135 - DNA, 14 bp, synthetic construct
  source 1..14: mol_type = other DNA
cacgtactaatgtc

SEQ ID NO: 136 - DNA, 14 bp, synthetic construct
  source 1..14: mol_type = other DNA
gcttggcgaatttg

SEQ ID NO: 137 - DNA, 14 bp, synthetic construct
  source 1..14: mol_type = other DNA
cgcggtacccttgg

SEQ ID NO: 138 - DNA, 14 bp, synthetic construct
  source 1..14: mol_type = other DNA
tcgacgcggtaccc

SEQ ID NO: 139 - DNA, 14 bp, synthetic construct
  source 1..14: mol_type = other DNA
catcgacgcggtac

SEQ ID NO: 140 - DNA, 14 bp, synthetic construct
  source 1..14: mol_type = other DNA
agcatcgacgcggt

SEQ ID NO: 141 - DNA, 14 bp, synthetic construct
  source 1..14: mol_type = other DNA
ctagaactgagcac

SEQ ID NO: 142 - DNA, 14 bp, synthetic construct
  source 1..14: mol_type = other DNA
gattattctgacta

SEQ ID NO: 143 - DNA, 14 bp, synthetic construct
  source 1..14: mol_type = other DNA
ggatgagcaagatt

SEQ ID NO: 144 - DNA, 14 bp, synthetic construct
  source 1..14: mol_type = other DNA
agatgagggcttga

SEQ ID NO: 145 - DNA, 14 bp, synthetic construct
  source 1..14: mol_type = other DNA
ttggcagatgaggg

SEQ ID NO: 146 - DNA, 14 bp, synthetic construct
  source 1..14: mol_type = other DNA
ggcagttttggcag

SEQ ID NO: 147 - DNA, 14 bp, synthetic construct
  source 1..14: mol_type = other DNA
ttgtaagtctttgg

SEQ ID NO: 148 - DNA, 14 bp, synthetic construct
  source 1..14: mol_type = other DNA
ccgtttgtaagtct

SEQ ID NO: 149 - DNA, 14 bp, synthetic construct
  source 1..14: mol_type = other DNA
gctcccgtttgtaa

SEQ ID NO: 150 - DNA, 14 bp, synthetic construct
  source 1..14: mol_type = other DNA
gtacaatggggggt

SEQ ID NO: 151 - DNA, 14 bp, synthetic construct
  source 1..14: mol_type = other DNA
aaaggcagtctgta

SEQ ID NO: 152 - DNA, 14 bp, synthetic construct
  source 1..14: mol_type = other DNA
tctagcaaaggcag

SEQ ID NO: 153 - DNA, 14 bp, synthetic construct
  source 1..14: mol_type = other DNA
gcattttctagcaa

SEQ ID NO: 154 - DNA, 14 bp, synthetic construct
  source 1..14: mol_type = other DNA
tggctaaggtcatt

SEQ ID NO: 155 - DNA, 14 bp, synthetic construct
  source 1..14: mol_type = other DNA
ataccattccagtg

SEQ ID NO: 156 - DNA, 14 bp, synthetic construct
  source 1..14: mol_type = other DNA
tgtaagagcattat

SEQ ID NO: 157 - DNA, 14 bp, synthetic construct
  source 1..14: mol_type = other DNA
aagcttatttgatc

SEQ ID NO: 158 - DNA, 14 bp, synthetic construct
  source 1..14: mol_type = other DNA
gataatggtgaagg

SEQ ID NO: 159 - DNA, 14 bp, synthetic construct
  source 1..14: mol_type = other DNA
atactggccttttg

SEQ ID NO: 160 - DNA, 14 bp, synthetic construct
  source 1..14: mol_type = other DNA
atcatgtcacttta

SEQ ID NO: 161 - DNA, 14 bp, synthetic construct
  source 1..14: mol_type = other DNA
tgcaatcatgtcac

SEQ ID NO: 162 - DNA, 14 bp, synthetic construct
  source 1..14: mol_type = other DNA
catctattactcaa

SEQ ID NO: 163 - DNA, 14 bp, synthetic construct
  source 1..14: mol_type = other DNA
tggtcaaagaggtt

SEQ ID NO: 164 - DNA, 14 bp, synthetic construct
  source 1..14: mol_type = other DNA
gttggtgttattca

SEQ ID NO: 165 - DNA, 14 bp, synthetic construct
  source 1..14: mol_type = other DNA
cagttggtgttatt

SEQ ID NO: 166 - DNA, 14 bp, synthetic construct
  source 1..14: mol_type = other DNA
aagaacagttggtg

SEQ ID NO: 167 - DNA, 14 bp, synthetic construct
  source 1..14: mol_type = other DNA
atcattgggttatg

SEQ ID NO: 168 - DNA, 14 bp, synthetic construct
  source 1..14: mol_type = other DNA
aaaggcacatctga

SEQ ID NO: 169 - DNA, 14 bp, synthetic construct
  source 1..14: mol_type = other DNA
atataggttattag

SEQ ID NO: 170 - DNA, 14 bp, synthetic construct
  source 1..14: mol_type = other DNA
tataggcctcaagt

SEQ ID NO: 171 - DNA, 14 bp, synthetic construct
  source 1..14: mol_type = other DNA
gattgtattatagg

SEQ ID NO: 172 - DNA, 14 bp, synthetic construct
  source 1..14: mol_type = other DNA
gcagattgtattat

SEQ ID NO: 173 - DNA, 14 bp, synthetic construct
  source 1..14: mol_type = other DNA
agtagcagattgta

SEQ ID NO: 174 - DNA, 14 bp, synthetic construct
  source 1..14: mol_type = other DNA
gtaaagtagcagat

SEQ ID NO: 175 - DNA, 14 bp, synthetic construct
  source 1..14: mol_type = other DNA
tttatgaattagta

SEQ ID NO: 176 - DNA, 14 bp, synthetic construct
  source 1..14: mol_type = other DNA
cagttggttcctat

SEQ ID NO: 177 - DNA, 14 bp, synthetic construct
  source 1..14: mol_type = other DNA
caaggcagttggtt

SEQ ID NO: 178 - DNA, 14 bp, synthetic construct
  source 1..14: mol_type = other DNA
tgaaggcaaggcag

SEQ ID NO: 179 - DNA, 14 bp, synthetic construct
  source 1..14: mol_type = other DNA
actaggtcttgaag

SEQ ID NO: 180 - DNA, 14 bp, synthetic construct
  source 1..14: mol_type = other DNA
aagttactaggtct

SEQ ID NO: 181 - DNA, 14 bp, synthetic construct
  source 1..14: mol_type = other DNA
acgagatttataga

SEQ ID NO: 182 - DNA, 14 bp, synthetic construct
  source 1..14: mol_type = other DNA
aacacgagatttat

SEQ ID NO: 183 - DNA, 14 bp, synthetic construct
  source 1..14: mol_type = other DNA
gttaacacgagatt

SEQ ID NO: 184 - DNA, 14 bp, synthetic construct
  source 1..14: mol_type = other DNA
acttatctgattaa

SEQ ID NO: 185 - DNA, 14 bp, synthetic construct
  source 1..14: mol_type = other DNA
gtaaataaacaacg

SEQ ID NO: 186 - DNA, 14 bp, synthetic construct
  source 1..14: mol_type = other DNA
ttattaatcttgtt

SEQ ID NO: 187 - DNA, 14 bp, synthetic construct
  source 1..14: mol_type = other DNA
actggggtgattta

SEQ ID NO: 188 - DNA, 14 bp, synthetic construct
  source 1..14: mol_type = other DNA
attcatgtatatct

SEQ ID NO: 189 - DNA, 14 bp, synthetic construct
  source 1..14: mol_type = other DNA
gttcatgtatatct

SEQ ID NO: 190 - DNA, 14 bp, synthetic construct
  source 1..14: mol_type = other DNA
aattcatgtatatc

SEQ ID NO: 191 - DNA, 14 bp, synthetic construct
  source 1..14: mol_type = other DNA
agttcatgtatatc

SEQ ID NO: 192 - DNA, 8395 bp, synthetic construct
  source 1..8395: mol_type = other DNA
attagagaaaaaaggcgacaggcattctgaatagcaggagacagagacagaagtgatgtgtttgtaggaggcaagagcagttcattttgagtgagagatacagctagaaagataagtagcggtccgattgtgcaaggccatggataccaggttaaggactttggaatttatcctgggacgccagtgtgagttcttgaacagaaaagtccaggggcaagttcatcctactctcgagtctaatgatgatgctcttcagtatgttgaagaattaattttgcaattattaaatatgctatgccaagctcagccccgaagtgcttcagatgtagaggaacgtgttcaaaaaagtttccctcatccaattgataaatgggcaatagctgatgcccaatcagctattgaaaagaggaagcgaagaaaccctttatctctcccagtagaaaaaattcatcctttattaaaggaggtcctaggttataaaattgaccaccaggtttctgtttacatagtagcagtcttagaatacatttctgcagacattttaaagctggttgggaattatgtaagaaatatacggcattatgaaattacaaaacaagatattaaagtggcaatgtgtgctgacaaggtattgatggatatgtttcatcaagatgtagaagatattaatatattatctttaactgacgaagagccttccacctcaggagaacaaacttactatgatttggtaaaagcatttatggcagaaattcgacaatatataagggaactaaatctaattataaaagtttttagagagccctttgtctccaattcaaaattgttttcagctaatgatgtagaaaatatatttagtcgcatagtagatatacatgaacttagtgtaaagttactgggccatatagaagatacagtagaaatgacagatgaaggcagtccccatccactagtaggaagctgctttgaagacttagcagaggaactggcatttgatccatatgaatcgtatgctcgagatattttgcgacctggttttcatgatcgtttccttagtcagttatcaaagcctggggcagcactttatttgcagtcaataggcgaaggtttcaaagaagctgttcaatatgttttacccaggctgcttctggcccctgtttaccactgtctccattactttgaacttttgaagcagttagaagaaaaaagtgaagatcaagaagacaaggaatgtttaaaacaagcaataacagctttgcttaatgttcagagtggtatggaaaaaatatgttctaaaagtcttgcaaaacgaagactgagtgaatctgcatgtcggttttatagtcagcaaatgaaggggaaacaactagcaatcaagaagatgaacgagattcagaagaatattgatggttgggagggaaaagacattggacagtgttgtaatgaatttataatggaaggaactcttacacgtgtaggagccaaacatgagagacacatatttctctttgatggcttaatgatttgctgtaaatcaaatcatgggcagccaagacttcctggtgctagcaatgcagaatatcgtcttaaagaaaagttttttatgcgaaaggtacaaattaatgataaagatgacaccaatgaatacaagcatgcttttgaaataattttaaaagatgaaaatagtgttatattttctgccaagtcagctgaagagaaaaacaattggatggcagcattgatatctttacagtaccggagtacactggaaaggatgcttgatgtaacaatgctacaggaagagaaagaggagcagatgaggctgcctagtgctgatgtttatagatttgcagagcctgactctgaagagaatattatatttgaagagaacatgcagcccaaggctggaattccaattatcaaagcaggaactgttattaaacttatagagaggcttacgtaccatatgtacgcagatcccaattttgttcggacatttcttacaacatacagatccttttgcaaacctcaagaactactgagtcttataatagaaaggtttgaaattccagagcctgagccaacagaagctgatcgcatagctatagagaatggagatcaacccttgagtgcagaactgaaaagatttagaaaagaatatatacagcctgtgcaactgcgagtattaaatgtatgtcggcactgggtagagcaccacttctatgattttgaaagagatgcatatcttttgcaacgaatggaagaatttattggaacagtaagaggtaaagcaatgaaaaaatgggttgaatccatcactaaaataatccaaaggaaaaaaattgcaagagacaatggaccaggtcataatattacatttcagagttcacctcccacagttgagtggcatataagcagacctgggcacatagagacttttgacctgctcaccttacacccaatagaaattgctcgacaactcactttacttgaatcagatctataccgagctgtacagccatcagaattagttggaagtgtgtggacaaaagaagacaaagaaattaactctcctaatcttctgaaaatgattcgacataccaccaacctcactctgtggtttgagaaatgtattgtagaaactgaaaatttagaagaaagagtagctgtggtgagtcgaattattgagattctacaagtctttcaagagttgaacaactttaatggtgtccttgaggttgtcagtgctatgaattcatcacctgtttacagactagaccacacatttgagcaaataccaagtcgccagaagaaaattttagaagaagctcatgaattgagtgaagatcactataagaaatatttggcaaaactcaggtctattaatccaccatgtgtgcctttctttggaatttatctcactaatatcttgaaaacagaagaaggcaaccctgaggtcctaaaaagacatggaaaagagcttataaactttagcaaaaggaggaaagtagcagaaataacaggagagatccagcagtaccaaaatcagccttactgtttacgagtagaatcagatatcaaaaggttctttgaaaacttgaatccgatgggaaatagcatggagaaggaatttacagattatcttttcaacaaatccctagaaatagaaccacgaaaccctaagcctctcccaagatttccaaaaaaatatagctatcccctaaaatctcctggtgttcgtccatcaaacccaagaccaggtaccatgaggcatcccacacctctgcagcaggagccaaggaaaattagttatagtaggatccctgaaagtgaaacagaaagtacagcatctgcaccaaattctccaagaacaccgttaacacctccgcctgcttctggtgcttccagtaccacagatgtttgcagtgtatttgattccgatcattcgagcccttttcactcaagcaatgataccgtctttatccaagttactctgccccatggcccaagatctgcttctgtatcatctataagtttaaccaaaggcactgatgaagtgcctgtccctcctcctgttcctccacgaagacgaccagaatctgccccagcagaatcttcaccatctaagattatgtctaagcatttggacagtcccccagccattcctcctaggcaacccacatcaaaagcctattcaccacgatattcaatatcagaccggacctctatctcagaccctcctgaaagccctcccttattaccaccacgagaacctgtgaggacacctgatgttttctcaagctcaccactacatctccaacctccccctttgggcaaaaaaagtgaccatggcaatgccttcttcccaaacagcccttccccctttacaccacctcctcctcaaacaccttctcctcacggcacaagaaggcatctgccatcaccaccattgacacaagaagtggaccttcattccattgctgggccgcctgttcctccacgacaaagcacttctcaacatatccctaaactccctccaaaaacttacaaaagggagcacacacacccatccatgcacagagatggaccaccactgttggagaatgcccattcttcctgagttcctctgtactgggatgtatattttcctagccccaaatccattgctggcaatggatgcactgaatgtgccagcactgaggagttaaaatgagaactccaaacactaacgactcttcttcaagatgcagtataagacaatgaattttaacctagatgtaattatacaatggaaatggtattccagtttagaatatggaaagaccgacctagaggaattggacaactgattgcacctgaaaatcataaagggactttttctggccaataggcaggagtcctctttttgtgaagtgatcttttgtgaagtgatcattaaagggatggaaaacacagtctagtgtccagcaggcccacatgacagtttttgtaattcaaattatgcacttttaaaaaaaaaaacttaaacagggatcttaatatcttcctttgttttcctttgctttactcttctactttagaatattttcttaaaaatcactcaaaggactgtgaggaaaggctgtggtacctgaccttgttgaaatcaaggcccggcactgtactacaggcctgtttacagattattacggtgaactgaatgggtaccgaggcttcaccaaagaggtacttttttgttgttgttgttgttttaggaataattgtaccaattttaagagcattccccccacctgtccccacacacccaaacaaaatgtggtggtgttgccttcaaaaaagagaagttttgtgtcattaacatgacagaagaactttttaaaaaaaaataactgtcaactattctatttgcatttaggagactgttcatctatgctagattgtcattttccctccttctcccacagaagtttactggtagtccatgtcatggctcgtagctatccctctaaccataccatggaaatgcaggcacccaatgtgaaaaggagcacttgctgggcatcactgacaccgctcatgttttacacatagttgagtaatcagcatatctagaattatcttgcattgcctaaatcatatgtatatagtgaatgttatataatatacctggcaggtctgttttaatttaattgaataaagatacaaatactttgtttggctggcatatattaaattattatatgaagaaaatggttgattcttatttcttttagttgaaaaacacacaaaaacactaaacataaaagcacgttttgtggtacgccttggtttctagttctggatatatgtattaatcttaaagaatgatctaaagtcacagcttggcatataggaagatacttggttagaataggatgggtatcttttaagcagaactttgtaagtaagttttaagttcctaagcaacaagtatacagtttgttaaggtttatattgaacattctaccttgcttgaggttttctgctttatcaaaactattcataatagaacatctacctgcatcattactatcataatatggattattcataaagcgtatgtactgttcttctgagtctaacaaatactgtgactgaaatgcatgcaccttgttgaagagaaaacattttattaatctccactataatgtctgtgaaatgtgcagtattccatccttagagagtttatctggttcatcagcatctgcatgtatcctctgaataatgaagcaacatttcagcaattctgttaaccacaccattagagacatttttagaagtgctgcaacatggtgggaacctagtactttaataatttcctatgatatttattagagcataaaatgtacatcacgatagtacagtatgtagtcctttaagaccagaaggtatgttaactgcaccatcattaagcagtctatcctattttcttatttatttgttagaattgaatcctttctcaatatctgtaccattcctatgatggtagccattcttctctttataaaatatttcctctttgaaaagcgtcctaaaacattaatttcaaatcccctcttggatgcaaagtgtccctaagaccatccattcaacagtatttatgtgagccaacttactagagccgctacaactaatcatgatagacagtccagaatctccctggactttatactgggccagcctcattgtccctaagatttctctttttatgtctcatggattacttctctttttaattgccccaggcacattccaaatgagaacctggtaaaagtcacattggacccccttcagtatgtcttcggctaatgtgttagccttcaataaggtgataaccccttttcaacctatctagtctataaagaggaaaaattcagatttggagttctaatcctatttctcccaaagggaactattcttctaccccatggttctaggagaccacttcactgtgcaacagttttacatgcattgtggatggaggatatttttattctgaactgttacatttaggtaaaaatattttctgcagtggtaactgattagaaaaatgccaattggatgcccttaggtggaggtgagaaaatggcatccttgccttcttctcaatatgaaacattaactagttgacaaatttatccttgtagtaatgaaaatctatttaatcagggaccagaaatggctgaggagataaatgcatcattacaaaattctgcttttgaatcctggacattacaagggggtaaatgcagcatgactttttgttaaccacattccaaaatgtggaacatttcttttagaaatgaaaatatttcaaggctgatgtattttaagactacacattatcaggaaacatacattgagagttcgcttaattaaaggttgttgggcatcaaattatgtttagtaggttactattctctaacaactcaaggatgctttaatggatctgaatttgacaaagagcatgccacactaatactacagtcaacaacagcccagagaacaattactatgtcagctggaggctatattatgattctaaattcttaaaggtttttttccctccataaatcaaaaattaccttatgtaaaccaaaaattagttggtatttatggtcatgatcttaattctcaagtttagcttaatcttgtatttcattgtttgtcttctaatatgacagcttaaattcagatttttaagtgactcagcaaaataggaggagtgtcccaatttattagtgttgtacatattgaagaaaacctttttgttccttcagatttagaaagaaacagtttaaccatttatttcttggtattctgctgctgtgaataggtttacttattcttttaacatatattgtgtgactacgcaagtataggtcctgttgtattgcattaatctttaccagtaactaaacatcacaaggttaatattggtttggctgaaagaattatgcagtaaagttatttataagggaacatgatgactttattcaatatttttcttctttgaaacatctcattactaacttttaagattatttcataatcccttatacatgagccaatgaaatattttgagctctacttaagaagcatgaagtctatattataaatctaaacaacaaaagcacttgtaacttgtttagtaaattccatgccttattttccatttttgacaccgtaaagtgcattttctgtcgactgtgagcaaatttcccttttgcctttaatgaaaatagtgcattaagtagccaattgctgcagaattataaagcaaattagctaacagtgggtcacattggactaccactcaaagaaacaagatgcataacagatgatggaaggtacatgtggtgtggcttagggagagcgcacctgtgccagatgcatgccactttcagtagctgccaaatgtgcctttaattaagaacatctctaatttttttcttcagatcaagtcagcatttggggtcgggatttgggcagggaactttggggtgcaaatatctggtgaaaccaccagagaatttgtttgtcaaagtgacatgtataattttagttgagacgtgtttgtgtatatatgtgtatttgcctttgtttagtagcacggttatttgctaaaataattgtcaaaaaattgctctgtcggtcggtctttgatgtaagaacaaagtgtggtctatacatctaatatggcctttgtacctaaccatgttcataggtaatctttgtactctgtgtgcagcagtatttggtttgcatttaaagtgaaaataacggctgttatttttctggcattactaagataaactgaaaaataataaagaaaaatgccttacatagcccacaa

SEQ ID NO: 193 - DNA, 8861 bp, synthetic construct
  source 1..8861: mol_type = other DNA
agtagcgagcaggttcagttcgctagtaggaagctccagcgctacaccggcggcggcggtggcggcggcggccgcggcggcacctcagggcagccagattccccaacaacgacctccggtccttccccttccccgcgagacctccgccccagctccgccccgcccggggctcttgcgtttcggagtcccaactacgcggagcgggcgccgcggccggagcgtccctccacacgtcctcccccagccgccagctccgccgcggggccagaccgggcccgggagtgcagccccggcgggcgggcggcggccgcggcagagggcagcccggggcggcgccggcgggccggggaggggcggggggcctggccccggggcggtggtgcgggatgccgccgcctccgggccgcgtccgttctccacggctggtacctgtgtcgggtgggtggccaggcgcgggcctcgccccccagccccctcgccagggctagcccggctgcgcggcgcccggagggggccgggccgtccggtggggccgcggccctgttccgcgctgcgagctcgccctctcgcggctccctggcccggccgccgccgcccctctccccgcccagaggcgccccgggggcaccatgcaggcgcagcagctgccctacgagtttttcagcgaagagaacgcgcccaagtggcggggactactggtgcctgcgctgaaaaaggtccaggggcaagttcatcctactctcgagtctaatgatgatgctcttcagtatgttgaagaattaattttgcaattattaaatatgctatgccaagctcagccccgaagtgcttcagatgtagaggaacgtgttcaaaaaagtttccctcatccaattgataaatgggcaatagctgatgcccaatcagctattgaaaagaggaagcgaagaaaccctttatctctcccagtagaaaaaattcatcctttattaaaggaggtcctaggttataaaattgaccaccaggtttctgtttacatagtagcagtcttagaatacatttctgcagacattttaaagctggttgggaattatgtaagaaatatacggcattatgaaattacaaaacaagatattaaagtggcaatgtgtgctgacaaggtattgatggatatgtttcatcaagatgtagaagatattaatatattatctttaactgacgaagagccttccacctcaggagaacaaacttactatgatttggtaaaagcatttatggcagaaattcgacaatatataagggaactaaatctaattataaaagtttttagagagccctttgtctccaattcaaaattgttttcagctaatgatgtagaaaatatatttagtcgcatagtagatatacatgaacttagtgtaaagttactgggccatatagaagatacagtagaaatgacagatgaaggcagtccccatccactagtaggaagctgctttgaagacttagcagaggaactggcatttgatccatatgaatcgtatgctcgagatattttgcgacctggttttcatgatcgtttccttagtcagttatcaaagcctggggcagcactttatttgcagtcaataggcgaaggtttcaaagaagctgttcaatatgttttacccaggctgcttctggcccctgtttaccactgtctccattactttgaacttttgaagcagttagaagaaaaaagtgaagatcaagaagacaaggaatgtttaaaacaagcaataacagctttgcttaatgttcagagtggtatggaaaaaatatgttctaaaagtcttgcaaaacgaagactgagtgaatctgcatgtcggttttatagtcagcaaatgaaggggaaacaactagcaatcaagaagatgaacgagattcagaagaatattgatggttgggagggaaaagacattggacagtgttgtaatgaatttataatggaaggaactcttacacgtgtaggagccaaacatgagagacacatatttctctttgatggcttaatgatttgctgtaaatcaaatcatgggcagccaagacttcctggtgctagcaatgcagaatatcgtcttaaagaaaagttttttatgcgaaaggtacaaattaatgataaagatgacaccaatgaatacaagcatgcttttgaaataattttaaaagatgaaaatagtgttatattttctgccaagtcagctgaagagaaaaacaattggatggcagcattgatatctttacagtaccggagtacactggaaaggatgcttgatgtaacaatgctacaggaagagaaagaggagcagatgaggctgcctagtgctgatgtttatagatttgcagagcctgactctgaagagaatattatatttgaagagaacatgcagcccaaggctggaattccaattatcaaagcaggaactgttattaaacttatagagaggcttacgtaccatatgtacgcagatcccaattttgttcggacatttcttacaacatacagatccttttgcaaacctcaagaactactgagtcttataatagaaaggtttgaaattccagagcctgagccaacagaagctgatcgcatagctatagagaatggagatcaacccttgagtgcagaactgaaaagatttagaaaagaatatatacagcctgtgcaactgcgagtattaaatgtatgtcggcactgggtagagcaccacttctatgattttgaaagagatgcatatcttttgcaacgaatggaagaatttattggaacagtaagaggtaaagcaatgaaaaaatgggttgaatccatcactaaaataatccaaaggaaaaaaattgcaagagacaatggaccaggtcataatattacatttcagagttcacctcccacagttgagtggcatataagcagacctgggcacatagagacttttgacctgctcaccttacacccaatagaaattgctcgacaactcactttacttgaatcagatctataccgagctgtacagccatcagaattagttggaagtgtgtggacaaaagaagacaaagaaattaactctcctaatcttctgaaaatgattcgacataccaccaacctcactctgtggtttgagaaatgtattgtagaaactgaaaatttagaagaaagagtagctgtggtgagtcgaattattgagattctacaagtctttcaagagttgaacaactttaatggtgtccttgaggttgtcagtgctatgaattcatcacctgtttacagactagaccacacatttgagcaaataccaagtcgccagaagaaaattttagaagaagctcatgaattgagtgaagatcactataagaaatatttggcaaaactcaggtctattaatccaccatgtgtgcctttctttggaatttatctcactaatatcttgaaaacagaagaaggcaaccctgaggtcctaaaaagacatggaaaagagcttataaactttagcaaaaggaggaaagtagcagaaataacaggagagatccagcagtaccaaaatcagccttactgtttacgagtagaatcagatatcaaaaggttctttgaaaacttgaatccgatgggaaatagcatggagaaggaatttacagattatcttttcaacaaatccctagaaatagaaccacgaaaccctaagcctctcccaagatttccaaaaaaatatagctatcccctaaaatctcctggtgttcgtccatcaaacccaagaccaggtaccatgaggcatcccacacctctgcagcaggagccaaggaaaattagttatagtaggatccctgaaagtgaaacagaaagtacagcatctgcaccaaattctccaagaacaccgttaacacctccgcctgcttctggtgcttccagtaccacagatgtttgcagtgtatttgattccgatcattcgagcccttttcactcaagatctgcttctgtatcatctataagtttaaccaaaggcactgatgaagtgcctgtccctcctcctgttcctccacgaagacgaccagaatctgccccagcagaatcttcaccatctaagattatgtctaagcatttggacagtcccccagccattcctcctaggcaacccacatcaaaagcctattcaccacgatattcaatatcagaccggacctctatctcagaccctcctgaaagccctcccttattaccaccacgagaacctgtgaggacacctgatgttttctcaagctcaccactacatctccaacctccccctttgggcaaaaaaagtgaccatggcaatgccttcttcccaaacagcccttccccctttacaccacctcctcctcaaacaccttctcctcacggcacaagaaggcatctgccatcaccaccattgacacaagaagtggaccttcattccattgctgggccgcctgttcctccacgacaaagcacttctcaacatatccctaaactccctccaaaaacttacaaaagggagcacacacacccatccatgcacagagatggaccaccactgttggagaatgcccattcttcctgagttcctctgtactgggatgtatattttcctagccccaaatccattgctggcaatggatgcactgaatgtgccagcactgaggagttaaaatgagaactccaaacactaacgactcttcttcaagatgcagtataagacaatgaattttaacctagatgtaattatacaatggaaatggtattccagtttagaatatggaaagaccgacctagaggaattggacaactgattgcacctgaaaatcataaagggactttttctggccaataggcaggagtcctctttttgtgaagtgatcttttgtgaagtgatcattaaagggatggaaaacacagtctagtgtccagcaggcccacatgacagtttttgtaattcaaattatgcacttttaaaaaaaaaaacttaaacagggatcttaatatcttcctttgttttcctttgctttactcttctactttagaatattttcttaaaaatcactcaaaggactgtgaggaaaggctgtggtacctgaccttgttgaaatcaaggcccggcactgtactacaggcctgtttacagattattacggtgaactgaatgggtaccgaggcttcaccaaagaggtacttttttgttgttgttgttgttttaggaataattgtaccaattttaagagcattccccccacctgtccccacacacccaaacaaaatgtggtggtgttgccttcaaaaaagagaagttttgtgtcattaacatgacagaagaactttttaaaaaaaaataactgtcaactattctatttgcatttaggagactgttcatctatgctagattgtcattttccctccttctcccacagaagtttactggtagtccatgtcatggctcgtagctatccctctaaccataccatggaaatgcaggcacccaatgtgaaaaggagcacttgctgggcatcactgacaccgctcatgttttacacatagttgagtaatcagcatatctagaattatcttgcattgcctaaatcatatgtatatagtgaatgttatataatatacctggcaggtctgttttaatttaattgaataaagatacaaatactttgtttggctggcatatattaaattattatatgaagaaaatggttgattcttatttcttttagttgaaaaacacacaaaaacactaaacataaaagcacgttttgtggtacgccttggtttctagttctggatatatgtattaatcttaaagaatgatctaaagtcacagcttggcatataggaagatacttggttagaataggatgggtatcttttaagcagaactttgtaagtaagttttaagttcctaagcaacaagtatacagtttgttaaggtttatattgaacattctaccttgcttgaggttttctgctttatcaaaactattcataatagaacatctacctgcatcattactatcataatatggattattcataaagcgtatgtactgttcttctgagtctaacaaatactgtgactgaaatgcatgcaccttgttgaagagaaaacattttattaatctccactataatgtctgtgaaatgtgcagtattccatccttagagagtttatctggttcatcagcatctgcatgtatcctctgaataatgaagcaacatttcagcaattctgttaaccacaccattagagacatttttagaagtgctgcaacatggtgggaacctagtactttaataatttcctatgatatttattagagcataaaatgtacatcacgatagtacagtatgtagtcctttaagaccagaaggtatgttaactgcaccatcattaagcagtctatcctattttcttatttatttgttagaattgaatcctttctcaatatctgtaccattcctatgatggtagccattcttctctttataaaatatttcctctttgaaaagcgtcctaaaacattaatttcaaatcccctcttggatgcaaagtgtccctaagaccatccattcaacagtatttatgtgagccaacttactagagccgctacaactaatcatgatagacagtccagaatctccctggactttatactgggccagcctcattgtccctaagatttctctttttatgtctcatggattacttctctttttaattgccccaggcacattccaaatgagaacctggtaaaagtcacattggacccccttcagtatgtcttcggctaatgtgttagccttcaataaggtgataaccccttttcaacctatctagtctataaagaggaaaaattcagatttggagttctaatcctatttctcccaaagggaactattcttctaccccatggttctaggagaccacttcactgtgcaacagttttacatgcattgtggatggaggatatttttattctgaactgttacatttaggtaaaaatattttctgcagtggtaactgattagaaaaatgccaattggatgcccttaggtggaggtgagaaaatggcatccttgccttcttctcaatatgaaacattaactagttgacaaatttatccttgtagtaatgaaaatctatttaatcagggaccagaaatggctgaggagataaatgcatcattacaaaattctgcttttgaatcctggacattacaagggggtaaatgcagcatgactttttgttaaccacattccaaaatgtggaacatttcttttagaaatgaaaatatttcaaggctgatgtattttaagactacacattatcaggaaacatacattgagagttcgcttaattaaaggttgttgggcatcaaattatgtttagtaggttactattctctaacaactcaaggatgctttaatggatctgaatttgacaaagagcatgccacactaatactacagtcaacaacagcccagagaacaattactatgtcagctggaggctatattatgattctaaattcttaaaggtttttttccctccataaatcaaaaattaccttatgtaaaccaaaaattagttggtatttatggtcatgatcttaattctcaagtttagcttaatcttgtatttcattgtttgtcttctaatatgacagcttaaattcagatttttaagtgactcagcaaaataggaggagtgtcccaatttattagtgttgtacatattgaagaaaacctttttgttccttcagatttagaaagaaacagtttaaccatttatttcttggtattctgctgctgtgaataggtttacttattcttttaacatatattgtgtgactacgcaagtataggtcctgttgtattgcattaatctttaccagtaactaaacatcacaaggttaatattggtttggctgaaagaattatgcagtaaagttatttataagggaacatgatgactttattcaatatttttcttctttgaaacatctcattactaacttttaagattatttcataatcccttatacatgagccaatgaaatattttgagctctacttaagaagcatgaagtctatattataaatctaaacaacaaaagcacttgtaacttgtttagtaaattccatgccttattttccatttttgacaccgtaaagtgcattttctgtcgactgtgagcaaatttcccttttgcctttaatgaaaatagtgcattaagtagccaattgctgcagaattataaagcaaattagctaacagtgggtcacattggactaccactcaaagaaacaagatgcataacagatgatggaaggtacatgtggtgtggcttagggagagcgcacctgtgccagatgcatgccactttcagtagctgccaaatgtgcctttaattaagaacatctctaatttttttcttcagatcaagtcagcatttggggtcgggatttgggcagggaactttggggtgcaaatatctggtgaaaccaccagagaatttgtttgtcaaagtgacatgtataattttagttgagacgtgtttgtgtatatatgtgtatttgcctttgtttagtagcacggttatttgctaaaataattgtcaaaaaattgctctgtcggtcggtctttgatgtaagaacaaagtgtggtctatacatctaatatggcctttgtacctaaccatgttcataggtaatctttgtactctgtgtgcagcagtatttggtttgcatttaaagtgaaaataacggctgttatttttctggcattactaagataaactgaaaaataataaagaaaaatgccttacatagcccacaa

SEQ ID NO: 194 - DNA, 8906 bp, synthetic construct
  source 1..8906: mol_type = other DNA
agtagcgagcaggttcagttcgctagtaggaagctccagcgctacaccggcggcggcggtggcggcggcggccgcggcggcacctcagggcagccagattccccaacaacgacctccggtccttccccttccccgcgagacctccgccccagctccgccccgcccggggctcttgcgtttcggagtcccaactacgcggagcgggcgccgcggccggagcgtccctccacacgtcctcccccagccgccagctccgccgcggggccagaccgggcccgggagtgcagccccggcgggcgggcggcggccgcggcagagggcagcccggggcggcgccggcgggccggggaggggcggggggcctggccccggggcggtggtgcgggatgccgccgcctccgggccgcgtccgttctccacggctggtacctgtgtcgggtgggtggccaggcgcgggcctcgccccccagccccctcgccagggctagcccggctgcgcggcgcccggagggggccgggccgtccggtggggccgcggccctgttccgcgctgcgagctcgccctctcgcggctccctggcccggccgccgccgcccctctccccgcccagaggcgccccgggggcaccatgcaggcgcagcagctgccctacgagtttttcagcgaagagaacgcgcccaagtggcggggactactggtgcctgcgctgaaaaaggtccaggggcaagttcatcctactctcgagtctaatgatgatgctcttcagtatgttgaagaattaattttgcaattattaaatatgctatgccaagctcagccccgaagtgcttcagatgtagaggaacgtgttcaaaaaagtttccctcatccaattgataaatgggcaatagctgatgcccaatcagctattgaaaagaggaagcgaagaaaccctttatctctcccagtagaaaaaattcatcctttattaaaggaggtcctaggttataaaattgaccaccaggtttctgtttacatagtagcagtcttagaatacatttctgcagacattttaaagctggttgggaattatgtaagaaatatacggcattatgaaattacaaaacaagatattaaagtggcaatgtgtgctgacaaggtattgatggatatgtttcatcaagatgtagaagatattaatatattatctttaactgacgaagagccttccacctcaggagaacaaacttactatgatttggtaaaagcatttatggcagaaattcgacaatatataagggaactaaatctaattataaaagtttttagagagccctttgtctccaattcaaaattgttttcagctaatgatgtagaaaatatatttagtcgcatagtagatatacatgaacttagtgtaaagttactgggccatatagaagatacagtagaaatgacagatgaaggcagtccccatccactagtaggaagctgctttgaagacttagcagaggaactggcatttgatccatatgaatcgtatgctcgagatattttgcgacctggttttcatgatcgtttccttagtcagttatcaaagcctggggcagcactttatttgcagtcaataggcgaaggtttcaaagaagctgttcaatatgttttacccaggctgcttctggcccctgtttaccactgtctccattactttgaacttttgaagcagttagaagaaaaaagtgaagatcaagaagacaaggaatgtttaaaacaagcaataacagctttgcttaatgttcagagtggtatggaaaaaatatgttctaaaagtcttgcaaaacgaagactgagtgaatctgcatgtcggttttatagtcagcaaatgaaggggaaacaactagcaatcaagaagatgaacgagattcagaagaatattgatggttgggagggaaaagacattggacagtgttgtaatgaatttataatggaaggaactcttacacgtgtaggagccaaacatgagagacacatatttctctttgatggcttaatgatttgctgtaaatcaaatcatgggcagccaagacttcctggtgctagcaatgcagaatatcgtcttaaagaaaagttttttatgcgaaaggtacaaattaatgataaagatgacaccaatgaatacaagcatgcttttgaaataattttaaaagatgaaaatagtgttatattttctgccaagtcagctgaagagaaaaacaattggatggcagcattgatatctttacagtaccggagtacactggaaaggatgcttgatgtaacaatgctacaggaagagaaagaggagcagatgaggctgcctagtgctgatgtttatagatttgcagagcctgactctgaagagaatattatatttgaagagaacatgcagcccaaggctggaattccaattatcaaagcaggaactgttattaaacttatagagaggcttacgtaccatatgtacgcagatcccaattttgttcggacatttcttacaacatacagatccttttgcaaacctcaagaactactgagtcttataatagaaaggtttgaaattccagagcctgagccaacagaagctgatcgcatagctatagagaatggagatcaacccttgagtgcagaactgaaaagatttagaaaagaatatatacagcctgtgcaactgcgagtattaaatgtatgtcggcactgggtagagcaccacttctatgattttgaaagagatgcatatcttttgcaacgaatggaagaatttattggaacagtaagaggtaaagcaatgaaaaaatgggttgaatccatcactaaaataatccaaaggaaaaaaattgcaagagacaatggaccaggtcataatattacatttcagagttcacctcccacagttgagtggcatataagcagacctgggcacatagagacttttgacctgctcaccttacacccaatagaaattgctcgacaactcactttacttgaatcagatctataccgagctgtacagccatcagaattagttggaagtgtgtggacaaaagaagacaaagaaattaactctcctaatcttctgaaaatgattcgacataccaccaacctcactctgtggtttgagaaatgtattgtagaaactgaaaatttagaagaaagagtagctgtggtgagtcgaattattgagattctacaagtctttcaagagttgaacaactttaatggtgtccttgaggttgtcagtgctatgaattcatcacctgtttacagactagaccacacatttgagcaaataccaagtcgccagaagaaaattttagaagaagctcatgaattgagtgaagatcactataagaaatatttggcaaaactcaggtctattaatccaccatgtgtgcctttctttggaatttatctcactaatatcttgaaaacagaagaaggcaaccctgaggtcctaaaaagacatggaaaagagcttataaactttagcaaaaggaggaaagtagcagaaataacaggagagatccagcagtaccaaaatcagccttactgtttacgagtagaatcagatatcaaaaggttctttgaaaacttgaatccgatgggaaatagcatggagaaggaatttacagattatcttttcaacaaatccctagaaatagaaccacgaaaccctaagcctctcccaagatttccaaaaaaatatagctatcccctaaaatctcctggtgttcgtccatcaaacccaagaccaggtaccatgaggcatcccacacctctgcagcaggagccaaggaaaattagttatagtaggatccctgaaagtgaaacagaaagtacagcatctgcaccaaattctccaagaacaccgttaacacctccgcctgcttctggtgcttccagtaccacagatgtttgcagtgtatttgattccgatcattcgagcccttttcactcaagcaatgataccgtctttatccaagttactctgccccatggcccaagatctgcttctgtatcatctataagtttaaccaaaggcactgatgaagtgcctgtccctcctcctgttcctccacgaagacgaccagaatctgccccagcagaatcttcaccatctaagattatgtctaagcatttggacagtcccccagccattcctcctaggcaacccacatcaaaagcctattcaccacgatattcaatatcagaccggacctctatctcagaccctcctgaaagccctcccttattaccaccacgagaacctgtgaggacacctgatgttttctcaagctcaccactacatctccaacctccccctttgggcaaaaaaagtgaccatggcaatgccttcttcccaaacagcccttccccctttacaccacctcctcctcaaacaccttctcctcacggcacaagaaggcatctgccatcaccaccattgacacaagaagtggaccttcattccattgctgggccgcctgttcctccacgacaaagcacttctcaacatatccctaaactccctccaaaaacttacaaaagggagcacacacacccatccatgcacagagatggaccaccactgttggagaatgcccattcttcctgagttcctctgtactgggatgtatattttcctagccccaaatccattgctggcaatggatgcactgaatgtgccagcactgaggagttaaaatgagaactccaaacactaacgactcttcttcaagatgcagtataagacaatgaattttaacctagatgtaattatacaatggaaatggtattccagtttagaatatggaaagaccgacctagaggaattggacaactgattgcacctgaaaatcataaagggactttttctggccaataggcaggagtcctctttttgtgaagtgatcttttgtgaagtgatcattaaagggatggaaaacacagtctagtgtccagcaggcccacatgacagtttttgtaattcaaattatgcacttttaaaaaaaaaaacttaaacagggatcttaatatcttcctttgttttcctttgctttactcttctactttagaatattttcttaaaaatcactcaaaggactgtgaggaaaggctgtggtacctgaccttgttgaaatcaaggcccggcactgtactacaggcctgtttacagattattacggtgaactgaatgggtaccgaggcttcaccaaagaggtacttttttgttgttgttgttgttttaggaataattgtaccaattttaagagcattccccccacctgtccccacacacccaaacaaaatgtggtggtgttgccttcaaaaaagagaagttttgtgtcattaacatgacagaagaactttttaaaaaaaaataactgtcaactattctatttgcatttaggagactgttcatctatgctagattgtcattttccctccttctcccacagaagtttactggtagtccatgtcatggctcgtagctatccctctaaccataccatggaaatgcaggcacccaatgtgaaaaggagcacttgctgggcatcactgacaccgctcatgttttacacatagttgagtaatcagcatatctagaattatcttgcattgcctaaatcatatgtatatagtgaatgttatataatatacctggcaggtctgttttaatttaattgaataaagatacaaatactttgtttggctggcatatattaaattattatatgaagaaaatggttgattcttatttcttttagttgaaaaacacacaaaaacactaaacataaaagcacgttttgtggtacgccttggtttctagttctggatatatgtattaatcttaaagaatgatctaaagtcacagcttggcatataggaagatacttggttagaataggatgggtatcttttaagcagaactttgtaagtaagttttaagttcctaagcaacaagtatacagtttgttaaggtttatattgaacattctaccttgcttgaggttttctgctttatcaaaactattcataatagaacatctacctgcatcattactatcataatatggattattcataaagcgtatgtactgttcttctgagtctaacaaatactgtgactgaaatgcatgcaccttgttgaagagaaaacattttattaatctccactataatgtctgtgaaatgtgcagtattccatccttagagagtttatctggttcatcagcatctgcatgtatcctctgaataatgaagcaacatttcagcaattctgttaaccacaccattagagacatttttagaagtgctgcaacatggtgggaacctagtactttaataatttcctatgatatttattagagcataaaatgtacatcacgatagtacagtatgtagtcctttaagaccagaaggtatgttaactgcaccatcattaagcagtctatcctattttcttatttatttgttagaattgaatcctttctcaatatctgtaccattcctatgatggtagccattcttctctttataaaatatttcctctttgaaaagcgtcctaaaacattaatttcaaatcccctcttggatgcaaagtgtccctaagaccatccattcaacagtatttatgtgagccaacttactagagccgctacaactaatcatgatagacagtccagaatctccctggactttatactgggccagcctcattgtccctaagatttctctttttatgtctcatggattacttctctttttaattgccccaggcacattccaaatgagaacctggtaaaagtcacattggacccccttcagtatgtcttcggctaatgtgttagccttcaataaggtgataaccccttttcaacctatctagtctataaagaggaaaaattcagatttggagttctaatcctatttctcccaaagggaactattcttctaccccatggttctaggagaccacttcactgtgcaacagttttacatgcattgtggatggaggatatttttattctgaactgttacatttaggtaaaaatattttctgcagtggtaactgattagaaaaatgccaattggatgcccttaggtggaggtgagaaaatggcatccttgccttcttctcaatatgaaacattaactagttgacaaatttatccttgtagtaatgaaaatctatttaatcagggaccagaaatggctgaggagataaatgcatcattacaaaattctgcttttgaatcctggacattacaagggggtaaatgcagcatgactttttgttaaccacattccaaaatgtggaacatttcttttagaaatgaaaatatttcaaggctgatgtattttaagactacacattatcaggaaacatacattgagagttcgcttaattaaaggttgttgggcatcaaattatgtttagtaggttactattctctaacaactcaaggatgctttaatggatctgaatttgacaaagagcatgccacactaatactacagtcaacaacagcccagagaacaattactatgtcagctggaggctatattatgattctaaattcttaaaggtttttttccctccataaatcaaaaattaccttatgtaaaccaaaaattagttggtatttatggtcatgatcttaattctcaagtttagcttaatcttgtatttcattgtttgtcttctaatatgacagcttaaattcagatttttaagtgactcagcaaaataggaggagtgtcccaatttattagtgttgtacatattgaagaaaacctttttgttccttcagatttagaaagaaacagtttaaccatttatttcttggtattctgctgctgtgaataggtttacttattcttttaacatatattgtgtgactacgcaagtataggtcctgttgtattgcattaatctttaccagtaactaaacatcacaaggttaatattggtttggctgaaagaattatgcagtaaagttatttataagggaacatgatgactttattcaatatttttcttctttgaaacatctcattactaacttttaagattatttcataatcccttatacatgagccaatgaaatattttgagctctacttaagaagcatgaagtctatattataaatctaaacaacaaaagcacttgtaacttgtttagtaaattccatgccttattttccatttttgacaccgtaaagtgcattttctgtcgactgtgagcaaatttcccttttgcctttaatgaaaatagtgcattaagtagccaattgctgcagaattataaagcaaattagctaacagtgggtcacattggactaccactcaaagaaacaagatgcataacagatgatggaaggtacatgtggtgtggcttagggagagcgcacctgtgccagatgcatgccactttcagtagctgccaaatgtgcctttaattaagaacatctctaatttttttcttcagatcaagtcagcatttggggtcgggatttgggcagggaactttggggtgcaaatatctggtgaaaccaccagagaatttgtttgtcaaagtgacatgtataattttagttgagacgtgtttgtgtatatatgtgtatttgcctttgtttagtagcacggttatttgctaaaataattgtcaaaaaattgctctgtcggtcggtctttgatgtaagaacaaagtgtggtctatacatctaatatggcctttgtacctaaccatgttcataggtaatctttgtactctgtgtgcagcagtatttggtttgcatttaaagtgaaaataacggctgttatttttctggcattactaagataaactgaaaaataataaagaaaaatgccttacatagcccacaa

SEQ ID NO: 195 - DNA, 5508 bp, synthetic construct
  source 1..5508: mol_type = other DNA
agaagcgggccagcgccgccgggaaaggaggtcgccgcccggggtcgcccgggcttgcgggtcgccgcgcttcctctgcgggcgctgcgggaggcgagggccgcctcggcggaggaagccgaccccggggtctccgccagcccgccggcgccgcctccgagccgcctcggccgtagcctcggcccgggcgggaggcggcggccgccggcgaggcgaggcggcccgcgccctgcctgtcaggccaccggcccggcccgcgggctgcccccgatgcggaggcgctgtcgccggggccatgcagcaggcgccgcagccttacgagttcttcagcgaggagaacagtccgaaatggcggggactgttggtctcggccctgcggaaggttcaggaacaagtgcatcccactctctcagctaatgaagagtctctctattatattgaagagctgatttttcagctgcttaataaattatgcatggcccagccaaggactgttcaagatgtagaggagcgagttcagaagacctttcctcacccaattgataaatgggccattgctgatgcacaatctgctatagaaaaacgaaaacgaagaaatcctcttttactgcctgtggacaaaatccatccttcgttgaaggaagtattagggtacaaagtggactaccatgtatccctatatattgtggctgtactagagtatatctcagctgatattttaaaattggctggtaattatgtttttaatatccggcattatgaaatatctcagcaggacattaaagtgtcaatgtgtgcggataaggttttgatggacatgtttgatcaggatgacataggtttggtttctctctgtgaagatgaacctagttcttctggtgaattaaactactatgatcttgtcagaactgaaatcgcagaagaaagacagtatctacgggaattaaatatgatcataaaagtgtttcgagaagcctttctttctgatagaaagctgtttaaaccttctgatatcgaaaagatttttagtaacatttcagatatacatgaattgactgtgaaacttttaggtttgattgaagacacagttgaaatgactgatgaaagcagtcctcatcccttagctggcagctgttttgaagatttggcagaagagcaagcatttgatccttatgaaacattatcacaggacattctttcaccagagtttcatgaacatttcaataaattgatggccagacctgcagttgctctacactttcagtccattgctgatggttttaaagaggcagttcgttatgtccttccacgtcttatgctggtgccagtgtatcactgttggcactactttgagttactaaagcaattgaaagcatgtagtgaagaacaagaagacagagaatgtttgaaccaagctattactgctctcatgaatctccaaggtagcatggaccgaatttacaagcagtattcacctagacgtcgacctggagatcctgtttgccctttttatagtcaccaattaagaagcaaacacctggctatcaaaaaaatgaatgaaattcagaaaaatatcgatggatgggaaggcaaagatattggacagtgttgtaatgaattcattatggagggaccattgacaagaatcggtgccaaacatgaacggcatatttttctgtttgatggcttaatgatcagttgtaaacctaatcatggccagactcggcttccaggttacagtagtgcagaatacaggttaaaagaaaaatttgtcatgaggaaaatacaaatttgtgataaagaagatacttgtgagcacaagcatgcatttgaattagtatccaaagatgagaacagcataatatttgctgctaagtctgctgaagaaaaaaacaactggatggcagcccttatttctcttcattatcgtagtactctagatcgaatgttagattcagtattattgaaagaagaaaatgagcaaccactgagattaccaagtcctgaagtatatcgttttgtagtaaaagactctgaggaaaacattgtttttgaagacaacttgcaaagtagaagtggcatccccattattaaaggaggaactgtagtgaaattaattgaaaggttaacatatcatatgtatgcagatcccaattttgttcgtacttttcttaccacatatcgttcattttgtaaaccacaggaattgctgagcttactgattgaacggtttgaaattccagagccagaacctactgacgcagacaaattggcaatagagaaaggcgagcagccaatcagtgcagaccttaaaagatttcgcaaggaatatgtccaaccagtacaacttaggatcttaaatgtatttcggcattgggttgaacatcatttttatgactttgaaagagacttggaattgcttgaaagactagaatccttcatttcaagtgtaagagggaaagctatgaaaaaatgggtagagtcaattgctaagatcatcaggaggaagaagcaagctcaggcaaacggagtaagccataatattacctttgaaagtccacctccaccaattgaatggcatatcagcaaaccaggacagtttgaaacatttgatctcatgacacttcatccaatagaaattgcacgtcagctgacacttttggagtctgatctttacaggaaagttcaaccgtctgaacttgtagggagtgtgtggaccaaagaagataaagaaataaattctccaaatttattaaaaatgattcgccataccacaaatctcaccctctggtttgaaaaatgcattgtggaagcagaaaattttgaagaacgggtggcagtactaagtagaattatagaaattctgcaagtttttcaagatttgaataatttcaatggcgtattggagatagtcagtgcagtaaattcagtgtcagtatacagactagaccatacctttgaggcactgcaggaaaggaaaaggaaaattttggacgaagctgtggaattaagtcaagatcactttaaaaaatacctagtaaaacttaagtcaatcaatccaccttgtgtgcctttttttggaatatatttaacaaatattctgaagaccgaagaagggaataatgattttttaaaaaagaaagggaaagatttaatcaatttcagtaagaggaggaaagtagctgaaattactggagaaattcagcagtatcagaatcagccttactgtttacggatagaaccagatatgaggagattctttgaaaaccttaaccccatgggaagtgcatctgaaaaagagtttacagattatttgttcaacaagtcactagaaattgaacctcgaaactgcaaacagccacctcgatttcctaggaaatcaactttttccttaaaatctcctggaataaggcctaacacaggccgacatggctctacctcaggtactttacgaggtcacccaacaccattagaaagagaaccatgtaaaataagctttagtcggattgctgaaactgagctggaatcaacagtgtcagcaccaacctctccaaatacaccatctactccaccagtatctgcttcttcagaccttagtgtatttttagatgtggatctcaacagctcctgtggcagcaatagcatctttgctccagtgcttttgccacattcaaagtctttctttagttcatgtggtagtttacataaactaagtgaagagcccctgattcctcctcctcttcctcctcgaaaaaagtttgatcatgatgcttcaaattccaagggaaatatgaaatctgatgatgatcctcctgctattccaccgagacagcctcctcctccaaaggtaaaacccagagttcctgttcctactggtgcatttgatgggcctctgcatagtccacctccgccaccaccaagagatcctcttcctgatacccctccaccagttccccttcggcctccagaacactttataaactgtccatttaatcttcagccacctccactggggcatcttcacagagattcagactggctcagagacattagtacgtgtccaaattcgccaagcactcctcctagcacaccctctccaagggtaccgcgtcgatgctatgtgctcagttctagtcagaataatcttgctcatcctccagctccccctgttccaccaaggcagaattcaagccctcatctgccaaaactgccaccaaagacttacaaacgggagctttcgcaccccccattgtacagactgcctttgctagaaaatgcagaaactccccaatgaccttagccatatgtagtcattgacactggaatggtatttgtaaagttttttttttaatttattcaaaaaaagacatagtattttagtactttttacaaataatgctcttacaaaaatgctactgatcaaataagcttttaagaattggaaaaataaaaatacaaaagtccttcaccattatcctcaggtgatcagtagcattgccttgtcttggatcctcagtgctgccaaaaggccagtataaagaatttatatttgcactgtaaactctgcaaaaatatggtttaaagtgacatgattgcactgaaaagggatagtgcttttgtgaaatttttcaaatttgagtaatagatgcctttttaaggcagtgaatttacacaaatatgggaggggtatatggtgttactgatttttaaacctctttgaccatcttctagtttttacttctagtttttacatctaggagaattgtgaataacaccaactgttcttaaactctttaatgccatgtcttaaatgccggtatttgctgctgaagacaaaaatgaaaagtaggatgaaaataatagaatggcactgtaagtgtttattattttgtcaaaatgtaaacaaagactacataacccaatgatggagggaaaaagggcatgtatctcattcagatgtgccttttgtttttgcagactatgacgtctttagctaatgaattgcctattgttatggaaaacagttaatatgccatgtatgtacagttttgtttatattgtatatttaaagatactgctaataacctatataaatttaagtgacttgaggcctataatacaatctgctactttactaattcataaattcaaaaaaaattctatggcataggaaccaactgccttgccttcaagacctagtaactttctctataaatctcgtgttaactgaaattttttttaaatatattttttagattggtaatatttaaaccagcaaatacttaaagctttattaaacattttaatcagataagtgagtaaagcttttatttgccatttggatgccttcgttcaaagtgatagagtgttttgctgatagtgctgtagcagcagttgtaaagtagccaaaagccacgttgtttatttactggtttgtggccttttactgtgctttgtatcagagttcttaacaagattaataaatcaccccagtcttaatttttaaaa